(19) United States
(12) Patent Application Publication
(10) Pub. No.: US 2025/0288672 A1
(43) Pub. Date: Sep. 18, 2025

(54) HUMANIZED SYNTHETIC NOTCH RECEPTORS WITH AUGMENTED TRANSACTIVATION DOMAINS AND USES THEREOF

(71) Applicants: Raymond LIU, San Francisco, CA (US); The Regents of the University of California, Oakland, CA (US)

(72) Inventors: Raymond LIU, San Francisco, CA (US); Dan PIRANER, San francisco, CA (US); Kole T. ROYBAL, San Francisco, CA (US)

(21) Appl. No.: 18/551,935

(22) PCT Filed: Mar. 23, 2022
(86) PCT No.: PCT/US22/21607

§ 371 Date: Sep. 22, 2023

Related U.S. Application Data
(60) Provisional application No. 63/165,525, filed on Mar. 24, 2021.

(51) Int. Cl. A61K 40/31; A61K 35/17; A61K 40/11; A61P 35/00; C07K 14/705; C07K 16/28; C07K 16/40; C12N 5/0783

(52) U.S. Cl. CPC A61K 40/31; A61K 35/17; A61K 40/11; A61P 35/00; C07K 14/705; C07K 16/2896; C07K 16/40; C12N 5/0636; A61K 2239/13; A61K 2239/21; A61K 2239/22; C07K 2317/53; C07K 2317/622; C12N 2510/00

(57) ABSTRACT

The present disclosure relates generally to chimeric antigen receptors (e.g., the synthetic Notch receptors disclosed herein) that contain two or more human or humanized transcriptional regulators to augment their function to regulate cell transcription. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

### CROSS REFERENCE TO RELATED APPLICATIONS

This application claims the benefit of U.S. Provisional Application No. 63/165,525, filed Mar. 24, 2021, which is incorporated herein by reference in entirety and for all purposes.

### STATEMENT REGARDING FEDERALLY SPONSORED R&D

This invention was made with government support under grant nos. OD025751 and R01 EB029483 awarded by The National Institutes of Health. The government has certain rights in the invention.

### INCORPORATION OF THE SEQUENCE LISTING

This application contains a Sequence Listing, which is hereby incorporated herein by reference in its entirety. The contents of the electronic sequence listing 048536-701001WO_Sequence_Listing_ST25.TXT; Size: 464,308 bytes; and Date of Creation: May 3, 2022.

### FIELD

The present disclosure relates generally to chimeric antigen receptors (e.g., the synthetic Notch receptors disclosed herein) that contain two or more human or humanized transcriptional regulators to augment their function to regulate cell transcription. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

### BACKGROUND

Notch receptors are transmembrane proteins that mediate cell-cell contact signaling and play a central role in development and other aspects of cell-to-cell communication. Notch receptors are involved in and are required for a variety of cellular functions during development, and are important for the function of a vast number of cell-types across species.

A number of first-generation synthetic derivatives of Notch receptors, which are often referred to as “SynNotch,” employ this straightforward signaling behavior by replacing the extracellular ligand-binding domain, which in wild-type Notch contains multiple EGF-like repeats, with an antibody derivative, and replacing the cytoplasmic domain with a transcription activator of choice, but still relying on the Notch negative regulatory region (NRR) (L. Morsut et al., Cell (2016) 164:780-91) and the standard two-step proteolysis described above. Additionally, the NRR spans approximately 160 amino acids, making this domain alone about three times the size of some mature proteins, such as insulin or epidermal growth factor (EGF). This makes expression of the receptor less efficient, and can exceed the capacity of some widely used cloning and transfection vectors.

In addition, these first-generation SynNotch and second-generation SynNotch receptors (e.g. HingeNotch etc.), in contrast to chimeric antigen receptor (CARs), do not elicit membrane proximal signaling via kinase cascades. The receptors, instead, have relatively weak capacities to induce transcription, thus being insufficient to activate cells to elicit a therapeutic effect. Therefore, there is an unmet need to modify SynNotch receptors and improve their functions for therapeutic uses.

### SUMMARY

The present disclosure provides compositions and methods of producing or using chimeric antigen receptors (e.g., the synthetic Notch receptors disclosed herein) that contain two or more transcriptional regulators to augment their function to regulate cell transcription and/or cell activation.

In one aspect, the present disclosure provides exemplary chimeric receptors (e.g., SynNotch receptors) that contain an intracellular domain (ICD) comprising two or more transcriptional regulators. In some embodiments, at least one of the two or more transcriptional regulators is human or humanized. In some embodiments, at least two of the two or more transcriptional regulators are human or humanized. In some embodiments, each one of the two or more transcriptional regulators is human or humanized.

Optionally, exemplary chimeric receptors may further incorporate an activation domain (e.g., the cytoplasmic tail of the CD3ζ chain) of a CAR to produce a SynNotch CAR receptor. The architecture of the cytoplasmic tail of these new receptors can be configured in multiple ways.

In some embodiments, the exemplary chimeric receptors include a chimeric receptor containing, from N-terminus to C-terminus:
an extracellular ligand-binding domain having a binding affinity for a ligand;
a linking polypeptide capable of promoting oligomer formation of the chimeric polypeptide via intermolecular disulfide bonding;
a transmembrane domain comprising one or more ligand-inducible proteolytic cleavage sites; and
an intracellular domain comprising two or more human or humanized transcriptional regulators.

In some embodiments, the exemplary chimeric receptors include a chimeric receptor containing, from N-terminus to C-terminus:
an extracellular ligand-binding domain having a binding affinity for a ligand;
a linking polypeptide capable of promoting oligomer formation of the chimeric polypeptide via intermolecular disulfide bonding;
a transmembrane domain comprising one or more ligand-inducible proteolytic cleavage sites; and
an intracellular domain comprising two or more human or humanized transcriptional regulators, wherein binding of the ligand to the extracellular ligand-binding domain induces cleavage at a ligand-inducible proteolytic cleavage site between the transcriptional regulator and the linking polypeptide, and
wherein the chimeric polypeptide does not comprise a LIN-12-Notch repeat (LNR) and/or a heterodimerization domain (HD) of a Notch receptor.

In some embodiments, the extracellular ligand-binding domain of an exemplary chimeric receptor described herein contains an antigen-binding moiety capable of binding to the ligand on the surface of a cell. In some embodiments, the cell is a pathogenic cell. In some embodiments, the cell is a human cell (e.g., a tumor or cancer cell). In some embodiments, the cell is a non-human cell. In some embodiments, the cell is a mammalian cell. In some embodiments, the cell is a tumor or cancer cell. In some embodiments, the cell is a terminally differentiated cell.

In some embodiments, the ligand recognizable by the extracellular ligand-binding domain described herein contains a protein or a carbohydrate. In some embodiments, the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), blue fluorescent protein (BFP) enhanced green fluorescent protein (EGFP), and signal regulatory protein α (SIRPα). In some embodiments, the ligand is selected from cell surface receptors, adhesion proteins, integrins, mucins, lectins, tumor associated antigens, and tumor-specific antigens. In some embodiments, the ligand is a tumor-associated antigen or a tumor-specific antigen. In some embodiments, the tumor-associated antigen is CD19, MCAM, BCMA, CEA, HER2, MUC1, CD20, ALPPL2, SIRPα, or EGFR. In some embodiments, the tumor-associated antigen is CD19, MCAM, BCMA, HER2, or ALPPL2.

In some embodiments, the extracellular ligand-binding domain contains the ligand-binding portion of a receptor. In some embodiments, the extracellular ligand-binding domain contains an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 1 or 2. In some embodiments, the extracellular ligand-binding domain contains an amino acid sequence having at least 80% sequence identity to SEQ ID NO: 1 or 2.

In some embodiments, the antigen-binding moiety described herein is selected from the group consisting of an antibody, a nanobody, a diabody, a triabody, a minibody, an F(ab′)2 fragment, an F(ab)v fragment, a single chain variable fragment (scFv), a single domain antibody (sdAb), and a functional fragment thereof. In some embodiments, the antigen-binding moiety contains a scFv. In some embodiments, the antigen-binding moiety specifically binds to a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD117), MUC1, NCAM, PAP, PDGFR-β, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

In some embodiments, the linking polypeptide of the exemplary chimeric receptors described herein contains a hinge domain. In some embodiments, the hinge domain is derived from a CD8a hinge domain, a CD28 hinge domain, a CD152 hinge domain, a PD-1 hinge domain, a CTLA4 hinge domain, an OX40 hinge domain, an IgG1 hinge domain, an IgG2 hinge domain, an IgG3 hinge domain, and an IgG4 hinge domain, or a functional variant of any thereof. In some embodiments, the linking polypeptide is derived from a CD8a hinge domain or a functional variant thereof. In some embodiments, the linking polypeptide contains an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 3. In some embodiments, the linking polypeptide contains an amino acid sequence having at least 80% sequence identity to SEQ ID NO: 3.

In some embodiments, the one or more ligand-inducible proteolytic cleavage sites of the exemplary chimeric receptors described herein contain a gamma secretase cleavage site.

In some embodiments, the transmembrane domain (TMD) of the exemplary chimeric receptors described herein contains an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 4. In some embodiments, the TMD contains an amino acid sequence having at least 80% sequence identity to SEQ ID NO: 4.

In some embodiments, the transmembrane domain (TMD) of the exemplary chimeric receptors described herein further contains a stop-transfer-sequence (STS). In some embodiments, the STS contains an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 5, and 75-79. In some embodiments, the STS contains an amino acid sequence having at least 80% sequence identity to SEQ ID NO: 5, and 75-79.

In some embodiments, the transmembrane domain (TMD) of the exemplary chimeric receptors described herein further contains a RAM sequence. In some embodiments, the RAM contains an amino acid sequence having at least 50%, 60%, 65%, 70%, 75%, 80%, 85%, 90%, 91%, 92%, 93%, 94%, 95%, 96%, 97%, 98%, 99%, or 100% sequence identity to SEQ ID NO: 80-90. In some embodiments, the RAM contains an amino acid sequence having at least 80% sequence identity to SEQ ID NO: 80-90.

In some embodiments, the exemplary chimeric receptors described herein further contain at least one of (i) an intracellular signaling domain (SD) derived from a signaling molecule and (ii) an activation domain. In some embodiments, the exemplary chimeric receptors described herein further contain (i) an intracellular signaling domain (SD) derived from a signaling molecule and (ii) an activation domain, and wherein the signaling molecule comprises a class 1 or a class 3 human membrane protein. In some embodiments, the signaling molecule is selected from the group consisting of CD28, CD244, CD7, CD72, CXADR, HAVCR2, NKG2D, NTB-A, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, 4-1BB, OX40, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-P1A, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC. In some embodiments, the signaling molecule is selected from the list consisting of OX40, ICOS, 4-1BB, CTLA4, CD28, CD30, CD2, CD27, and CD226. In some embodiments, the optional activation domain contains one or more immunoreceptor tyrosine-based activation motifs (ITAMs), such as one or more ITAMs are derived from CD3ζ, CD3σ, CD3γ, and CD3C. In some embodiments, the one or more ITAMs are at least about 80, 85, 90, 95, 96, 97, 98, 99, or 100% identical to a CD3ζ ITAM. In some embodiments, the activation domain comprises an amino acid sequence having at least 80% sequence identity to a CD3ζ ITAM.

In some embodiments, the two or more human or humanized transcriptional regulators in the exemplary chimeric receptors described herein contain at least a transcriptional activator, or a transcriptional repressor. In some embodiments, the intracellular domain contains a transcriptional regulator sequence containing an transactivation domain (TAD) derived from a protein selected from the group consisting of VP64, p65, KRAB transactivating variants, and VP16. In some embodiments, the intracellular domain further contains a DNA binding domain derived from a protein selected from the group consisting of Gal4, tetR, ZFHID1, Zif268, and HAP1. In some embodiments, the exemplary chimeric receptors further contain an additional transcriptional regulator containing a TAD derived from the group consisting of HSF-1, GATA3, HIF1a, GR Tau1, ATF6, ELF3, MLXIPL, NFE2L2, MIER3, PTF1A, and p53.

In some embodiments, the exemplary chimeric receptors described herein contain an amino acid sequence having at least 50%, 60%, 65%, 70%, 75%, 80%, 85%, 90%, 91%, 92%, 93%, 94%, 95%, 96%, 97%, 98%, 99%, or 100% sequence identity to any one of SEQ ID NOs: 35-71. In some embodiments, the exemplary chimeric receptors described herein contain an amino acid sequence having at least 80% sequence identity to any one of SEQ ID NOs: 35-71, and 99-123.

In some embodiments, the exemplary chimeric receptors described herein further contains a signal sequence, a detectable label, a tumor-specific cleavage site, a disease-specific cleavage site, or a combination thereof.

In another aspect, the present disclosure further provides a recombinant nucleic acid containing a nucleotide sequence encoding any one of the exemplary chimeric receptors described herein. In some embodiments, the nucleotide sequence is incorporated into an expression cassette or an expression vector. In some embodiments, the expression vector is a viral vector. In some embodiments, the viral vector is a lentiviral vector, an adeno virus vector, an adeno-associated virus vector, or a retroviral vector.

In another aspect, the present disclosure further provides a recombinant cell containing any one of the exemplary chimeric receptor and/or the recombinant nucleic acid described herein. In some embodiments, the recombinant cell is a eukaryotic cell. In some embodiments, the eukaryotic cell is a mammalian cell. In some embodiments, the mammalian cell is an immune cell, a neuron, an epithelial cell, and endothelial cell, or a stem cell. In some embodiments, the immune cell is a B cell, a monocyte, a natural killer cell, a basophil, an eosinophil, a neutrophil, a dendritic cell, a macrophage, a regulatory T cell, a helper T cell, a cytotoxic T cell, or other T cells.

In some embodiments, the recombinant cell contains
a) a first chimeric receptor and a second chimeric receptor described herein; and/or
b) a first nucleic acid and a second nucleic acid described herein;
wherein the first chimeric receptor and the second chimeric receptor do not have the same sequence, and/or the first nucleic acid or the second nucleic acid do not have the same sequence.

In some embodiments, the first chimeric receptor modulates the expression and/or activity of the second chimeric receptor.

In some embodiments, the recombinant cell further contains an expression cassette encoding a polypeptide operably linked to a promoter, wherein expression of the polypeptide is modulated by the transcriptional regulator. In some embodiments, the polypeptide is heterologous to the cell. In some embodiments, the promoter is a yeast GAL4 promoter. In some embodiments, the polypeptide is a cytokine, a cytotoxin, a chemokine, an immunomodulator, a pro-apoptotic factor, an anti-apoptotic factor, a hormone, a differentiation factor, a de-differentiation factor, an immune cell receptor (e.g., a TCR or CAR), or a reporter.

In another aspect, the present disclosure further provides a method for making the recombinant cell described herein, comprising:
a) providing a cell capable of protein expression;
b) contacting the provided cell with a recombinant nucleic acid described herein into the provided cell.

In some embodiments, the cell is obtained by leukapheresis performed on a sample obtained from a subject, and the cell is contacted ex vivo.

In some embodiments, the recombinant nucleic acid is encapsulated in a viral capsid or a lipid nanoparticle.

In another aspect, the present disclosure further provides a pharmaceutical composition comprising a pharmaceutically acceptable carrier, and one or more of the following: the recombinant nucleic acid described herein; and the recombinant cell described herein.

In some embodiments, the composition comprises a recombinant nucleic acid described herein, and a pharmaceutically acceptable carrier.

In some embodiments, the recombinant nucleic acid is encapsulated in a viral capsid or a lipid nanoparticle.

In another aspect, the present disclosure further provides a system for modulating an activity of a cell, inhibiting a target cancer cell, or treating a health condition in an individual in need thereof, the system containing one or more of the following:
a chimeric receptor described herein;
a recombinant nucleic acid described herein;
a recombinant cell described herein; and
a pharmaceutical composition described herein.

In another aspect, the present disclosure further provides a method for modulating an activity of a cell, the method including:
a) providing a recombinant cell described herein; and
b) contacting the recombinant cell with a selected ligand, wherein binding of the selected ligand to the extracellular ligand-binding domain induces cleavage of a ligand-inducible proteolytic cleavage site and releases the transcriptional regulator, wherein the released transcriptional regulator modulates an activity of the recombinant cell.

In some embodiments, the contacting in b) is carried out in vivo, ex vivo, or in vitro.

In some embodiments, the activity of the cell to be modulated is selected from the group consisting of: expression of a selected gene, proliferation, apoptosis, non-apoptotic death, differentiation, dedifferentiation, migration, secretion of a molecule, cellular adhesion, and cytolytic activity.

In some embodiments, the released transcriptional regulator modulates expression of a gene product of the cell. In some embodiments, the released transcriptional regulator modulates expression of a heterologous gene product.

In some embodiments, the gene product of the cell is selected from the group consisting of chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, an RNA guided nuclease, a site-specific nuclease, a T cell receptor, a toxin, a toxin derived protein, a transcriptional regulator, a transcriptional activator, a transcriptional repressor, a translational regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

In some embodiments, the released transcriptional regulator modulates differentiation of the cell, and wherein the cell is an immune cell, a stem cell, a progenitor cell, or a precursor cell.

In another aspect, the present disclosure further provides a method for inhibiting an activity of a target cell in an individual, the method including administering to the individual an effective number of the recombinant cells described herein, wherein the recombinant cells inhibit an activity of the target cell in the individual. In some embodiments, the target cell is a pathogenic cell. In some embodiments, the pathogenic cell is a cancer cell. In some embodiments, the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell.

In another aspect, the present disclosure further provides a method for the treatment of a health condition in an individual in need thereof, the method containing administering to the individual a first therapy containing an effective number of the recombinant cell described herein, wherein the recombinant cell treats the health condition in the individual. In some embodiments, the method further includes administering to the individual a second therapy. In some embodiments, the second therapy is selected from the group consisting of chemotherapy, radiotherapy, immunotherapy, hormonal therapy, and toxin therapy. In some embodiments, the first therapy and the second therapy are administered together in the same composition or in separate compositions. In some embodiments, the first therapy and the second therapy are administered at the same time. In some embodiments, the first therapy and the second therapy are administered sequentially. In some embodiments, the first therapy is administered before the second therapy. In some embodiments, the first therapy is administered after the second therapy. In some embodiments, the first therapy and the second therapy are administered in rotation.

In another aspect, the present disclosure further provides a use of one or more of the following for the treatment of a health condition:
a) a chimeric receptor described herein;
b) a recombinant nucleic acid described herein;
c) a recombinant cell described herein; and
d) a composition described herein.

In another aspect, the present disclosure further provides a use of one or more of the following for the manufacture of a medicament for the treatment of a health condition:
a) a chimeric receptor described herein;
b) a recombinant nucleic acid described herein;
c) a recombinant cell described herein; and
d) a composition described herein.

In some embodiments, the health condition is cancer. In some embodiments, the cancer is a solid tumor, a soft tissue tumor, or a metastatic lesion.

### BRIEF DESCRIPTION OF THE DRAWINGS

FIGS. 1A-1C are a set of graphs showing the design and tested functions of HingeNotch receptors containing a transcriptional effector. FIG. 1A shows a sequence alignment of mouse and human p65 transactivation domains. A SynNotch receptor was previously prepared to contain a mouse p65-human HSF1 fusion protein. Such construct was further humanized by fusing human HSF1 TAD to either the full-length human p65 TAD or to the human “mouse-homologous region” (MHR). Anti-CD19 HingeNotch receptors were produced by fusing the zinc figure protein 3 (ZF3) DNA binding domain to transactivation domains consisting of the mouse p65 TAD, the human p65 MHR, human p65 MHR fused to HSF1 TAD, or full-length human p65 fused to HSF1 TAD. BFP activation from a ZF3-responsive cassette in primary human CD4+ T cells, introduced with various anti-CD19 HingeNotch receptors, was examined at 48 hours post-stimulation with CD19+ or CD19− K562 cells (FIG. 1). FIG. 1C shows quantification of % BFP-positive (left panel) and activated MFI (right panel) of the experiment shown in FIG. 1B. Activated MFI is defined as the mean fluorescence intensity of all cells brighter than the untransduced control.

FIGS. 2A-2B are a set of graphs showing the design and tested functions of HingeNotch receptors containing the zinc finger protein 6 (ZF6) DNA binding domain and various transcriptional effectors. FIG. 2A compared the expression of various HingeNotch receptors in CD4+ T cells incubated with control K562 cells (left panel) or CD19-positive K562 cells (right panels). FIG. 2B shows quantification of % BFP reporter-positive cells (left panel) and reporter MFI (right panel) for the data in FIG. 2A.

FIGS. 3A-3B are a set of graphs showing the target cell killing of T cells expressing HingeNotch receptors containing various transcriptional effectors. FIG. 3A shows FACS histograms for cell scatter profiles of co-cultured T cells and K562 cells. Primary human CD8+ T cells were co-transduced with a lentiviral vector bearing a zinc finger-inducible anti-MCAM CAR fused to a 2A-GFP sequence, followed by a constitutive PGK promoter driving BFP expression, along with a panel of lentiviral vectors containing a PGK promoter driving myc-tagged anti-ALPPL2 HingeNotch variants bearing a set of transcription factors, including TADs consisting of p65 TAD, p65 TAD-HSF1 TAD, and p65 TAD-GR1. Co-transduced T cells were isolated by cell sorting for Myc/BFP double-positive events. Five days after dynabead removal and sorting, the T cells were co-incubated with K562 cells expressing MCAM, ALPPL2, or both antigens for 72 hours before measurement. FIG. 3B is a graph comparing numbers of live K562 cells, after co-culturing with T cells expressing various HingeNotch receptors, detected by a DRAQ7 viability assay and FACS event counting.

FIGS. 4A-4C show in vivo efficacy of humanized ZFTF HingeNotches. FIG. 4A is an experimental layout of the in vivo test. K562s expressing BCMA (left) or BCMA and CD19 (right) were injected subcutaneously into mice. After the initiation of tumor outgrowth, primary human T-cells modified with a constitutive BCMA CAR or CD19 HingeNotch variants driving the BCMA CAR were injected retro-orbitally. FIG. 4B shows the experimental timeline. T-cells were injected 4 days after K562 injection, after which tumor volumes were measured. FIG. 4C shows tumor growth curves of BMCA-only tumors (L) or BMCA/CD19 tumors (R).

FIGS. 5A-5D show additional transactivation domain optimization. FIG. 5A shows a representation of ZF3 or ZF6 HingeNotch performance with single human-derived TADs replacing p65 (top) or p65-TAD fusions (bottom). Each vertical bar represents a receptor variant. Receptors with high inducibility are indicated in text. FIG. 5B shows sort efficiency of primary human T-cells bearing TAD-optimized ZF6 HingeNotches. FIG. 5C shows BFP activation profiles of the T-cells described in FIG. 5B. FIG. 5D shows MFI quantification of expression levels reported in FIG. 5C.

FIGS. 6A-6D show structural optimization of ZFTF-bearing HingeNotches. FIG. 6A shows a comparison of receptor performance with HingeNotch structural modifications. The original Notch2 STS/p65 receptor induction is shown alongside receptors bearing the p65-MLXIPL TAD and Notch1 or Notch2 STS, with and without an 8-residue sequence from the Notch1 RAM domain. All variants bear the SV40 nuclear localization sequence. FIG. 6B shows a comparison of HingeNotch activity between receptor variants with otherwise identical sequence bearing ZF6 SynTFs containing a nuclear localization sequence from SV40 or Notch1.

FIG. 6C shows a comparison of ZF6 HingeNotch activity bearing the p65 TAD (left) or the p65-MLXIPL TAD and a post-juxtamembrane domain corresponding to fragments of the human Notch1 RAM domain, or truncations upstream of the RAM start (RAM-5 to RAM-1). FIG. 6D shows BFP expression levels of primary human T-cells bearing p65, p65-MLXIPL (RAM1), or p65-MLXIPL (RAM8) ZF6 HingeNotch variants in the presence of K562 (−) or K562^{CD19+} (+) cells.

FIGS. 7A-7B show additional p65 TAD fusions testing with Pax6-based receptors in a single vector format. FIG. 7A shows HingeNotch activity between receptor variants with otherwise identical sequence bearing different p65 variant and TAD combinations. HingeNotch receptors (anti-CD19scFv-hingeNotch-N2STS-Pax6-TAD) and response cassettes (Pax6RE>flag-anti-BCMAscFv-CAR) were combined into a single lentiviral vector for transduction into a pool of CD4+ and CD8+ T cells. Activity was measured by measuring surface expression of the inducible Flag-tagged BCMA CAR after three days of co-culture with K562-BCMA or K562-CD19-BCMA target cells. Notation: p65(Long) is p65(aa361-551) and p65(Short) is p65(428-551). FIG. 7B shows a three day time course of the Pax6-p65(Long)-Tau1-based receptor, tested as in (A) and examined after 24, 48 and 72 hours.

### DETAILED DESCRIPTION OF THE DISCLOSURE

The present disclosure provides exemplary chimeric receptors (e.g., SynNotch receptors) that contain an intracellular domain (ICD) comprising two or more human or humanized transcriptional regulators. Optionally, exemplary chimeric receptors may further incorporate an activation domain (e.g., the cytoplasmic tail of the CD3C chain) of a CAR to produce a SynNotch CAR receptor. The architecture of the cytoplasmic tail of these new receptors can be configured in multiple ways. Further, the present disclosure identifies human or humanized receptor architectures that may reliably promote SynNotch signaling and gene expressions in primary human T cells. Multiple additional transcriptional effectors, when incorporated into human or humanized SynNotch receptors, were shown to improve activation and signaling of the SynNotch receptors in T cells, such as improving the expression of downstream target genes.

Although various features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In addition, the materials, methods, and examples are illustrative only and not intended to be limiting.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used herein, refer to the delivery of a composition or formulation by an administration route including, but not limited to, intravenous, intra-arterial, intracerebral, intrathecal, intramuscular, intraperitoneal, subcutaneous, intramuscular, and combinations thereof. The term includes, but is not limited to, administration by a medical professional and self-administration

The term “heterologous” refers to a polypeptide sequence or domain which is not native to a flanking sequence, e.g., wherein the heterologous sequence is not found in nature coupled to the polypeptide sequences occurring at one or both ends.

The term “derived from” as used herein in reference to a protein or polypeptide refers to an origin or source, and may include naturally occurring, recombinant, unpurified or purified polypeptide that is obtained from, is obtained based on a source or original protein or polypeptide. As such, a protein or polypeptide derived from an original protein or polypeptide may include the original protein or polypeptide, in part or in whole, and may be a fragment or variant of the original protein or polypeptide. In some instance, the polypeptide sequence or domain that is derived from a source or origin can be genetically or chemically modified.

The terms “host cell” and “recombinant cell” are used interchangeably herein. It is understood that such terms, as well as “cell”, “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the originally cell or cell line.

The term “operably linked”,” as used herein, denotes a physical or functional linkage between two or more elements, e.g., polypeptide sequences or polynucleotide sequences, which permits them to operate in their intended fashion.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI web site at ncbi.nlm.nih.gov/BLAST. Such sequences are then said to be “substantially identical.” This definition also refers to, or may be applied to, the complement of a test sequence. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al., Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, and unless otherwise specified, a “therapeutically effective amount” of an agent is an amount sufficient to provide a therapeutic benefit in the treatment or management of a health condition, such as a disease (e.g., a cancer), or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” is an individual under the care of a physician. Thus, the subject can be a human individual or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

One skilled in the art will understand that the chimeric receptors disclosed herein provide signals having a range of characteristics, from low to high ligand-induced transduction and (independently) low to moderate non-induced signal transduction. This range of activities is a new feature that can be exploited to enhance and tune the actions of engineered cells. Further, as described in greater detail below, a number of the receptor variants disclosed herein exhibit improved expression compared to existing SynNotch receptors.

### Notch Receptors

Notch receptors are large transmembrane proteins that normally communicate signals upon binding to surface-bound ligands expressed on adjacent cells. Notch signals rely on cell- to cell communication, e.g., communication between two contacting cells, in which one contacting cell is a “receiver” cell and the other contacting cell is a “sender” cell. Notch receptors expressed in a receiver cell recognize their ligands (the delta/serrate/lag, or “DSL” family of proteins) expressed on a sending cell. The engagement of notch and delta on these contacting cells leads to a two-step proteolysis of the notch receptor, which ultimately causes the release of the intracellular portion of the receptor (“ICD”) from the membrane into the cytoplasm. Notch has a matrix metalloprotease cleavage site (denoted “S2”), which, when the receptor is not activated is protected from cleavage by the Notch negative regulatory region (“NRR”). The NRR consists of three LIN-12-Notch repeat (“LNR”) modules and a heterodimerization domain (“HD”). It is believed that this proteolysis is regulated by the force exerted by the sending cell: the DSL ligand pulls on the Notch receptor, which changes the conformation of the NRR and exposes the metalloprotease site. This is cleaved by a constitutively active protease (such as ADAM10), which releases the extracellular binding portion and negative regulatory region of the receptor. Release of the ligand binding portion of the receptor in turn exposes another cleavage site (denoted “S3”), which is cleaved by γ-secretase within the cell membrane: this cleavage releases the nuclear homing ICD from the cell membrane. W. R. Gordon et al., Dev Cell (2015) 33:729-36. This released domain alters receiver cell behavior by regulating transcription. Evolutionary divergence of vertebrates and invertebrates was accompanied by at least two rounds of gene duplication in the Notch lineage: flies possess a single Notch gene, worms two (GLP-1 and LIN-12), and mammals four (NOTCH1-4). Transduction of Notch signals relies on three key events: (i) ligand recognition; (ii) conformational exposure of the ligand-dependent cleavage site; and (iii) assembly of nuclear transcriptional activation complexes.

Canonical Notch signals are transduced by a process called regulated intramembrane proteolysis. Notch receptors are normally maintained in a resting, proteolytically resistant conformation on the cell surface, but ligand binding initiates a proteolytic cascade that releases the intracellular domain of the receptor (ICD) from the membrane. The critical, regulated cleavage step is effected by ADAM metalloproteases and occurs at a site called S2 immediately external to the plasma membrane. This truncated receptor, dubbed NEXT (for Notch extracellular truncation), remains membrane-tethered until it is processed at site S3 by γ-secretase, a multiprotein enzyme complex.

After γ-secretase cleavage, the ICD ultimately enters the nucleus, where it nucleates assembly of a transcriptional activation complex that contains a DNA-binding transcription factor, and a transcriptional coactivator of the Mastermind family. This complex then engages one or more additional coactivator proteins such as p300 to recruit the basal transcription machinery and activate the expression of downstream target genes.

Notch receptors have a modular domain organization. The ectodomains of Notch receptors consist of a series of N-terminal epidermal growth factor (EGF)-like repeats that are responsible for ligand binding. O-linked glycosylation of these EGF repeats, including modification by O-fucose, Fringe, and Rumi glycosyltransferases, also modulates the activity of Notch receptors in response to different ligand subtypes in flies and mammals.

The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

### Compositions of the Disclosure

The present disclosure provides, among others, SynNotch receptor architectures that may augment custom transcriptional regulation (e.g., through a transcriptional regulation domain) by further incorporating an additional transcriptional regulator (e.g., a transcriptional effector) to modulate (e.g., to improve) the capacity of transcriptional regulation. The new receptors provided herein have linear amino acid signaling motif to mediate signaling in T cells added into the cytoplasmic tail of SynNotch receptors. In some embodiments, the linear amino acid signaling motif comprises one or more human or humanized sequences. As demonstrated in the Examples and figures, these new receptors have increased capacities to activate gene transcription capable of activating T cells.

In one aspect, the present disclosure provides chimeric receptor comprising, from N-terminus to C-terminus, one or more of the following components: a) an extracellular ligand-binding domain having a binding affinity for a selected ligand; b) a linking polypeptide; c) a transmembrane domain comprising one or more ligand-inducible proteolytic cleavage sites; and d) an intracellular domain comprising two or more human or humanized transcriptional regulators. In some embodiments, the linking polypeptide is capable of promoting oligomer formation of the chimeric receptor via intermolecular disulfide bonding. In some embodiments, the binding of the selected ligand to the extracellular ligand-binding domain induces cleavage at a ligand-inducible proteolytic cleavage site disposed between the transcriptional regulator (e.g., a transcriptional effector) and the linking polypeptide. In addition, the chimeric receptor provided herein does not comprise a LIN-12-Notch repeat (LNR) and/or a heterodimerization domain (HD) of a Notch receptor.

In some embodiments, a SynNotch receptor described herein further contains one or more signaling domains (e.g. CD28, 4-1BB, and/or CD3zeta, etc.) that can initiate activation of T cells concomitant with transcriptional regulation.

Chimeric polypeptides and SynNotch receptors of the present disclosure can be chimeric polypeptides of any length, including chimeric polypeptides that are generally between about 100 amino acids (aa) to about 1000 aa, e.g., from about 100 aa to about 200 aa, from about 150 aa to about 250 aa, from about 200 aa to about 300 aa, from about 250 aa to about 350 aa, from about 300 aa to about 400 aa, from about 350 aa to about 450 aa, from about 400 aa to about 500 aa in length. In some embodiments, the disclosed chimeric polypeptides are at least about 100, 200, 300, 400, 500, 600, 700, 750, 800, 850, 900, 950, or 1,000 aa in length. In some embodiments, the disclosed chimeric polypeptides are less than about 1,500, 1,400, 1,300, 1,200, 1,100, 1,000, 950, 900, 850, 800, 750, 700, 600, 500, 400, 350, 300, 250, or 200 aa in length. In some embodiments, the disclosed chimeric polypeptides are generally between about 400 aa to about 450 aa, from about 450 aa to about 500 aa, from about 500 aa to about 550 aa, from about 550 aa to about 600 aa, from about 600 aa to about 650 aa, from about 650 aa to about 700 aa, from about 700 aa to about 750 aa, from about 750 aa to about 800 aa, from about 800 aa to about 850 aa, from about 850 aa to about 900 aa, from about 900 aa to about 950 aa, or from about 950 aa to about 1000 aa in length. In some cases, the chimeric polypeptides of the present disclosure have a length of about 300 aa to about 400 aa. In some cases, the chimeric polypeptides of the present disclosure have a length of about 300 aa to about 350 aa. In some cases, the chimeric polypeptides of the present disclosure have a length of about 300 aa to about 325 aa. In some cases, the chimeric polypeptides of the present disclosure have a length of about 350 aa to about 400 aa. In some cases, the chimeric polypeptides of the present disclosure have a length of 750 aa to 850 aa.

### Extracellular Domains and Ligands

In some embodiments, the ECD of the chimeric receptors (e.g., SynNotch receptors) disclosed herein has a binding affinity for one or more target ligands. The terms “target ligand” and “target antigen” are used interchangeably in the present disclosure. The target ligand can be expressed on the surface of a cell, or is otherwise anchored, immobilized, or restrained so that it can exert a mechanical force on the chimeric receptor. The cell can be a pathogen or a human cell. In some embodiments, the human cell can be a tumor cell. In some embodiments, the human cell can be a terminally differentiated cell. As such, without being bound to any particular theory, binding of the ECD of a chimeric receptor provided herein to a cell-surface ligand does not necessarily remove the target ligand from the target cell surface, but instead enacts a mechanical pulling force on the chimeric receptor. For example, an otherwise soluble ligand may be targeted if it is bound to a surface, or to a molecule in the extracellular matrix. In some embodiments, the target ligand is a cell-surface ligand. Non-limiting examples of suitable ligand types include cell surface receptors; adhesion proteins; carbohydrates, lipids, glycolipids, lipoproteins, and lipopolysaccharides that are surface-bound; integrins; mucins; and lectins. In some embodiments, the ligand is a protein. In some embodiments, the ligand is a carbohydrate.

In some embodiments, the ligand is a cluster of differentiation (CD) marker. In some embodiments, the CD marker is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, and MAGE.

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment thereof. One skilled in the art upon reading the present disclosure will readily understand that the term “functional fragment thereof” or “functional variant thereof” refers to a molecule having quantitative and/or qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, a nanobody, a diabody, a triabody, or a minibody, an F(ab′)2 fragment, an F(ab) fragment, a single chain variable fragment (scFv), and a single domain antibody (sdAb), or a functional fragment thereof. In some embodiments, the antigen-binding moiety includes a scFv.

The antigen-binding moiety can include naturally-occurring amino acid sequences or can be engineered, designed, or modified to provide desired and/or improved properties such as, e.g., binding affinity. Generally, the binding affinity of an antigen-binding moiety, e.g., an antibody, for a target antigen (e.g., CD19 antigen) can be calculated by the Scatchard method described by Frankel et al., Mol. Immunol, 16:101-06, 1979. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. An antibody that “selectively binds” an antigen (such as CD19) is an antigen-binding moiety that does not significantly bind other antigens but binds the antigen with high affinity, e.g., with an equilibrium constant (KD) of 100 nM or less, such as 60 nM or less, for example, 30 nM or less, such as, 15 nM or less, or 10 nM or less, or 5 nM or less, or 1 nM or less, or 500 pM or less, or 400 pM or less, or 300 pM or less, or 200 pM or less, or 100 pM or less.

A skilled artisan can select an ECD based on the desired localization or function of a cell that is genetically modified to express a chimeric receptor of the present disclosure. For example, a chimeric receptor with an ECD including an antibody specific for a HER2 antigen can target cells to HER2-expressing breast cancer cells. In some embodiments, the ECD of the disclosed human or humanized SynNotch receptors is capable of binding a tumor-associated antigen (TAA) or a tumor-specific antigen (TSA). A skilled artisan will understand that TAAs include a molecule, such as e.g., protein, present on tumor cells and on normal cells, or on many normal cells, but at much lower concentration than on tumor cells. In contrast, TSAs generally include a molecule, such as e.g., protein which is present on tumor cells but absent from normal cells.

In some cases, the antigen-binding moiety is specific for an epitope present in an antigen that is expressed by a tumor cell, i.e., a tumor-associated antigen. The tumor-associated antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell, a non-Hodgkin B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a myelogenous leukemia cell, a neuroblastoma cell, a glioma, a glioblastoma, a colorectal cancer cell, etc. It will also be understood that a tumor-associated antigen may also be expressed by a non-cancerous cell. In some embodiments, the antigen-binding domain is specific for an epitope present in a tissue-specific antigen. In some embodiments, the antigen-binding domain is specific for an epitope present in a disease-associated antigen.

Non-limiting examples of suitable target antigens include CD19, B7H3 (CD276), BCMA (CD269), alkaline phosphatase, placental-like 2 (ALPPL2), green fluorescent protein (GFP), enhanced green fluorescent protein (EGFP), signal regulatory protein α (SIRPα), CD123, CD171, CD179α, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD 117), MUC1, NCAM, PAP, PDGFR-β, PRSS21, PSCA, PSMA, ROR1, SSEA-4, TAG72, TEM1/CD248, TEM7R, TSHR, VEGFR2, ALPI, citrullinated vimentin, cMet, and Axl.

In some embodiments, the target antigen is selected from CD19, B7H3 (CD276), BCMA (CD269), ALPPL2, CD123, CD171, CD179α, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD 117), MUC1, NCAM, PAP, PDGFR-β, PRSS21, PSCA, PSMA, ROR1, SSEA-4, TAG72, TEM1/CD248, TEM7R, TSHR, VEGFR2, ALPI, citrullinated vimentin, cMet, Axl, GPC2, human epidermal growth factor receptor 2 (Her2/neu), CD276 (B7H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD123, CD93, CD99, CD 117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, AOC3 (VAP-1), CAM-3001, CCL11 (eotaxin-1), CD125, CD147 (basigin), CD154 (CD40L), CD2, CD20, CD23 (IgE receptor), CD25 (a subunit of the heterodimeric IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGFβ1, TNF-α, VEGF-A, pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), and mesothelin.

Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), Ra1-B, GPC2, CD276 (B7H3), or IL-13Rα. In some embodiments, the antigen is Her2. In some embodiments, the antigen is ALPPL2. In some embodiments, the antigen is BCMA. In some embodiments, the antigen-binding moiety of the ECD is specific for a reporter protein, such as BFP, GFP, and eGFP. Non-limiting examples of such antigen-binding moiety include a LaG17 anti-GFP nanobody. In some embodiments, the antigen-binding moiety of the ECD includes an anti-BCMA fully-humanized VH domain (FHVH). In some embodiments, the antigen is signal regulatory protein α (SIRPα).

Additional antigens suitable for targeting by the chimeric receptors disclosed herein include, but are not limited to GPC2, human epidermal growth factor receptor 2 (Her2/neu), CD276 (B7H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD 117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Additional antigens suitable for targeting by the chimeric receptors disclosed herein include, but are not limited to, those associated with an inflammatory disease such as, AOC3 (VAP-1), CAM-3001, CCL11 (eotaxin-1), CD125, CD147 (basigin), CD154 (CD40L), CD2, CD20, CD23 (IgE receptor), CD25 (a subunit of the heteromeric of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGFβ1, TNF-α, and VEGF-A.

Further antigens suitable for targeting by the chimeric receptors disclosed herein include, but are not limited to the pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), and Ra1-B. In some embodiments, the antigen is GPC2, CD19, Her2/neu, CD276 (B7H3), IL-13Rα1, or IL-13Rα2. In some embodiments, the antigen is Her2. In some embodiments, the antigen is ALPPL2. In some embodiments, the antigen is BCMA. In some embodiments, the antigen-binding moiety of the ECD is specific for a reporter protein, such as GFP and eGFP. Non-limiting examples of such antigen-binding moiety include a LaG17 anti-GFP nanobody. In some embodiments, the antigen-binding moiety of the ECD includes an anti-BCMA fully-humanized VH domain (FHVH).

In some embodiments, antigens suitable for targeting by the chimeric receptors disclosed herein include ligands derived from a pathogen. For example, the antigen can be HER2 produced by HER2-positive breast cancer cells. In some embodiments, the antigen can be CD19 that is expressed on B-cell leukemia. In some embodiments, the antigen can be EGFR that is expressed on glioblastoma multiform (GBM) but much less expressed so on healthy CNS tissue. In some embodiments, the antigen can be CEA that is associated with cancer in adults, for example colon cancer.

In some embodiments, the antigen-binding moiety of the ECD is specific for a cell surface target, where non-limiting examples of cell surface targets include CD19, CD30, Her2, CD22, ENPP3, EGFR, CD20, CD52, CD11α, and α-integrin. In some embodiments, the chimeric receptors disclosed herein include an extracellular domain having an antigen-binding moiety that binds CD19, CEA, HER2, MUC1, CD20, ALPPL2, BCMA, or EGFR. In some embodiments, the chimeric receptors provided herein (e.g., human or humanized SynNotch receptors, such as HingeNotch receptors) include an extracellular domain including an antigen-binding moiety that binds CD19. In some embodiments, the chimeric receptors provided herein (e.g., human or humanized SynNotch receptors, such as HingeNotch receptors) include an extracellular domain including an antigen-binding moiety that binds ALPPL2. In some embodiments, the chimeric receptors provided herein (e.g., human or humanized SynNotch receptors, such as HingeNotch receptors) include an extracellular domain including an antigen-binding moiety that binds BCMA. In some embodiments, the chimeric receptors provided herein (e.g., human or humanized SynNotch receptors, such as HingeNotch receptors) include an extracellular domain including an antigen-binding moiety that binds Her2. In some embodiments, the chimeric receptors and human or humanized SynNotch receptors, such as HingeNotch receptors disclosed herein include an extracellular domain including an antigen-binding moiety that binds CD19, ALPPL2, BCMA, or Her2.

In some embodiments, the extracellular domain includes an amino acid sequence having at least 70%, at least 75%, at least 80%, at least 85%, at least 90%, at least 95%, at least 96%, at least 97%, at least 98%, at least 99% sequence identity to a sequence set forth in SEQ ID NO: 1 or 2. In some embodiments, the extracellular domain includes an amino acid sequence having at least 90% sequence identity to a sequence set forth in SEQ ID NO: 1 or 2. In some embodiments, the extracellular domain includes an amino acid sequence having at least 95% sequence identity to a sequence set forth in SEQ ID NO: 1 or 2. In some embodiments, the extracellular domain includes an amino acid sequence having 100% sequence identity to a sequence set forth in SEQ ID NO: 1 or 2. In some embodiments, the extracellular domain includes an amino acid sequence set forth in SEQ ID NO: 1 or 2, wherein one, two, three, four, or five of the amino acid residues in any one of the SEQ ID NO: 1 or 2 is/are substituted by a different amino acid residue.

### Linking Polypeptide

As described above, the chimeric receptors of the disclosure include a linking sequence disposed between the extracellular binding domain (ECD) and the transmembrane domain (TMD). Existing “SynNotch” receptors comprise a heterologous extracellular ligand-binding domain, a linking polypeptide having substantial sequence identity with a Notch receptor JMD including the NRR, a TMD, and an ICD. In contrast, the chimeric receptors described herein comprise a heterologous extracellular ligand-binding domain, a linking polypeptide having substantial sequence identity with a Notch receptor JMD but lacking the NRR (the LIN-12-Notch repeat (LNR) modules, and the heterodimerization domain), a TMD, and an ICD, while the ICD further has an additional transcriptional regulator (e.g., a transcriptional effector). Stated differently, in the human or humanized SynNotch receptors described herein, the linking polypeptide replaces the negative regulatory region (NRR) and heterodimerization (HD) domain of the native Notch.

In some embodiments, the length and amino acid composition of the linking polypeptide sequence can be optimized to vary the orientation and/or proximity of ECD and TMD relative to one another to achieve a desired activity of the chimeric polypeptides and receptors as disclosed herein. In some embodiments, the length and amino acid composition of the linking polypeptide sequence can be varied as a “tuning” tool to achieve a tuning effect that would enhance or reduce the biological activity of the disclosed chimeric polypeptides and receptors. Additional information regarding the relative glycine content, length, amino acid composition, and the flexibility/stiffness of glycerin-serine linking polypeptide can be determined by any methodologies known in the art as suitable for such purposes, for example as determined by Förster resonance energy transfer (FRET) efficiencies as described in Rosmalen M. et al., Biochemistry (2017), 56:6565-74.

In some embodiments, an single-chain peptide including about two to 100 amino acid residues (aa) e.g., 4, 5, 6, 7, 8, 9, 10, 11, 12, 13, 14, 15, 16, 17, 18, 19, 20, etc. amino acid residues) is used as a linking polypeptide in the disclosed chimeric receptors. In some embodiments, the linking polypeptide sequence has a length ranging from about 5 to about 50, about 10 to about 60, about 20 to about 70, about 30 to about 80, about 40 to about 90, about 50 to about 100, about 60 to about 80, about 70 to about 100, about 30 to about 60, about 20 to about 80, about 30 to about 90 amino acid residues. In some embodiments, the linking polypeptide sequence has a length ranging from about 1 to about 10, about 5 to about 15, about 10 to about 20, about 15 to about 25, about 20 to about 40, about 30 to about 50, about 40 to about 60, about 50 to about 70 amino acid residues. In some embodiments, the linking polypeptide sequence has a length ranging from about 40 to about 70, about 50 to about 80, about 60 to 8 about 0, about 70 to about 90, or about 80 to about 100 amino acid residues. In some embodiments, the linking polypeptide sequence has a length ranging from about 1 to about 10, about 5 to about 15, about 10 to about 20, about 15 to about 25 amino acid residues.

In some embodiments, the linking polypeptide sequence has a length ranging from about 1 to about 40 amino acid residues. In some embodiments, the linking polypeptide sequence has a length ranging from 1 to about 10, about 5 to about 20, about 10 to about 30, about 15 to about 40, about 10 to about 40, about 15 to about 40, about 20 to about 40, about 25 to about 40, about 30 to about 40, about 5 to about 0, about 15 to about 30 amino acid residues. In some embodiments, the linking polypeptide sequence has a length ranging from about 5 to about 40, about 10 to about 35, about 15 to about 35, about 20 to about 35, about 5 to about 20, about 5 to about 25, about 5 to about 30, about 5 to about 35 amino acid residues.

In certain embodiments, the linking polypeptide contains only glycine and/or serine residues (e.g., glycine-serine linking polypeptide). Examples of such linking polypeptides include: Gly, Ser; Gly Gly Ser; Ser Gly Gly; Gly Ser Gly; Gly Gly Gly Ser; Ser Gly Gly Gly; Ser Gly Ser Gly; Gly Gly Gly Gly Ser; Ser Gly Gly Gly Gly; Gly Gly Gly Gly Gly Ser; Ser Gly Gly Gly Gly Gly; Gly Gly Gly Gly Gly Gly Ser; Ser Gly Gly Gly Gly Gly Gly; (Gly Gly Gly Gly Ser)n, wherein n is an integer of one or more; and (Ser Gly Gly Gly Gly)n, wherein n is an integer of one or more. In some embodiments, the linking polypeptide sequence includes at least one glycine residue. In some embodiments, the linking polypeptide sequence includes at least one serine residue. In some embodiments, the linking polypeptide sequences are modified such that the amino acid sequence Gly Ser Gly (GSG) (that occurs at the junction of traditional Gly/Ser linker polypeptide repeats) is not present. For example, in some embodiments, the linking polypeptide includes an amino acid sequence selected from the group consisting of: (GGGXX)nGGGGS and GGGGS(XGGGS)n, where X is any amino acid that can be inserted into the sequence and not result in a polypeptide comprising the sequence GSG, and n is 0 to 4. In some embodiments, the sequence of a linking polypeptide is (GGGX1X2)nGGGGS and X1 is P and X2 is S and n is 0 to 4. In some embodiments, the sequence of a linking polypeptide is (GGGX1X2)nGGGGS and X1 is G and X2 is Q and n is 0 to 4. In some other embodiments, the sequence of a linking polypeptide is (GGGX1X2)nGGGGS and X1 is G and X2 is A and n is 0 to 4. In some embodiments, the sequence of a linking polypeptide is GGGGS(XGGGS)n, and X is P and n is 0 to 4. In some embodiments, a linking polypeptide of the disclosure comprises or consists of the amino acid sequence (GGGGA)2GGGGS. In some embodiments, a linking polypeptide comprises or consists of the amino acid sequence (GGGGQ)2GGGGS. In some embodiments, a linking polypeptide comprises or consists of the amino acid sequence (GGGPS)2GGGGS. In some embodiments, a linking polypeptide comprises or consists of the amino acid sequence GGGGS(PGGGS)2.

Three to 50 amino acid residues (e.g., 3, 4, 5, 6, 7, 8, 9, 10, 11, 12, 13, 14, 15, 16, 17, 18, 19, 20, etc. amino acid residues) can be used as a polypeptide linker. In some embodiments, the length and amino acid composition of the linker polypeptide sequence can be optimized to vary the orientation and/or proximity of the ECD and the TMD relative to one another to achieve a desired activity of the chimeric receptor of the disclosure. All of these sequences can be used as a linking polypeptide for the chimeric receptors of the present disclosure.

In some embodiments, the linking polypeptide encompassed by the present disclosure also includes a polypeptide capable of promoting oligomer formation of the chimeric receptor via intermolecular disulfide bonding, for example, hinge linkers. Hinge linkers of the disclosure include an oligomerization domain (e.g., a hinge domain) containing one or more polypeptide motifs that promote oligomer formation of the chimeric receptors via intermolecular disulfide bonding. In these instances, within the chimeric receptors disclosed herein, the hinge domain generally includes a flexible polypeptide connector region disposed between the ECD and the TMD. Thus, the hinge domain provides flexibility between the ECD and TMD and also provides sites for intermolecular disulfide bonding between two or more chimeric receptor monomers to form an oligomeric complex. In some embodiments, the hinge domain includes motifs that promote dimer formation of the chimeric receptors disclosed herein. In some embodiments, the hinge domain includes motifs that promote trimer formation of the chimeric receptors disclosed herein (e.g., a hinge domain derived from OX40). Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more CXXC motifs. In some embodiments, the hinge polypeptide sequence contains one or more CPPC motifs. Additional information in this regard can be found in, for example, a recent review by G. Vidarsson et al., Frontiers Immunol (2014) 5:520 (doi: 10.3389/fimmu.2014.00520).

Hinge polypeptide sequences can also be derived from a CD8a hinge domain, a CD28 hinge domain, a CD152 hinge domain, a PD-1 hinge domain, a CTLA4 hinge domain, an OX40 hinge domain, and functional variants thereof. In some embodiments, the hinge domain includes a hinge polypeptide sequence derived from a CD8a hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

The hinge linker can include about 5 to about 60 amino acids from or overlapping with the selected hinge domain, for example at least about 5, at least about 6, at least about 7, at least about 8, at least about 9, at least about 10, at least about 12, at least about 15, at least about 17, at least about 20, at least about 22, at least about 24, at least about 26, at least about 28, at least about 30, at least about 35, at least about 40, at least about 45, at least about 50, at least about 55, or, at least about 60 amino acids. In embodiments of the invention, the Hinge linker has no more than about 60 amino acids, less than about 55, less than about 50, less than about 45, less than about 40, less than about 35, less than about 32, less than about 30, less than about 29, less than about 28, less than about 27, less than about 26, less than about 25, less than about 24, less than about 23, less than about 22, less than about 21, less than about 20, less than about 18, less than about 16, less than about 14, less than about 12, or less than about 10 amino acids.

In some embodiments, the linking polypeptide sequence includes a sequence having at least 80% sequence identity, such as, at least 80%, at least 85%, at least 90%, at least 95%, at least 96%, at least 97%, at least 98%, or 99% sequence identity to a sequence set forth in SEQ ID NO: 3. In some embodiments, the linking polypeptide sequence includes an amino acid sequence having at least 90% sequence identity to a sequence set forth in SEQ ID NO: 3. In some embodiments, the linking polypeptide sequence includes an amino acid sequence having at least 95% sequence identity to a sequence set forth in SEQ ID NO: 3. In some embodiments, the linking polypeptide sequence includes an amino acid sequence having at least 99% sequence identity to a sequence set forth in SEQ ID NO: 3. In some embodiments, the linking polypeptide sequence includes an amino acid sequence identical to a sequence set forth in SEQ ID NO: 3. In some embodiments, the linking polypeptide sequence includes an amino acid sequence set forth in SEQ ID NO: 3, wherein one, two, three, four, or five of the amino acid residues in any one of the SEQ ID NO: 3 is/are substituted by a different amino acid residue.

### Transmembrane Domains (TMD)

As described above, the chimeric receptors of the disclosure include a TMD comprising one or more ligand-inducible proteolytic cleavage sites.

Examples of proteolytic cleavage sites in a Notch receptor (e.g., S2 or S3) are as described above. Additional proteolytic cleavage sites suitable for the compositions and methods disclosed herein include, but are not limited to, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue such as Leu, Ile, Val, Phe, Trp, Tyr, Val, Met, and Pro), e.g., Pro-X-X-Hy-(Ser/Thr), e.g., Pro-Leu/Gln-Gly-Met-Thr-Ser or Pro-Leu/Gln-Gly-Met-Thr. Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., Glu-Asn-Leu-Tyr-Thr-Gln-Ser, where the protease cleaves between the glutamine and the serine. Another example of a protease cleavage site that can be included in a proteolytically cleavable linker is an enterokinase cleavage site, e.g., Asp-Asp-Asp-Asp-Lys, where cleavage occurs after the lysine residue. Another example of a protease cleavage site that can be included in a proteolytically cleavable linker is a thrombin cleavage site, e.g., Leu-Val-Pro-Arg. Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr virus protease, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrix metalloproteinase (MT-MMP), stromelysin 3 (or MMP-11), thermolysin, fibroblast collagenase and stromelysin-1, matrix metalloproteinase 13 (collagenase-3), tissue-type plasminogen activator(tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

Generally, the TMD suitable for the chimeric receptors disclosed herein can be any transmembrane domain of a Type 1 transmembrane receptor including at least one γ-secretase cleavage site. Detailed description of the structure and function of the γ-secretase complex as well as its substrate proteins, including amyloid precursor protein (APP) and Notch, can, for example, be found in a recent review by Zhang et al., Frontiers Cell Neurosci (2014). Non-limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one γ-secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric receptors of the disclosure is a TMD derived from the TMD of a member of the calsyntenin family, such as, alcadein alpha and alcadein gamma. In some embodiments, the TMD of the chimeric receptors of the disclosure is a TMD known for Notch receptors. In some embodiments, the TMD of the chimeric receptors of the disclosure is a TMD derived from a different Notch receptor. For example, in a Mini Notch based on human Notch1, the Notch1 TMD can be substituted with a Notch2 TMD, Notch3 TMD, Notch4 TMD, or a Notch TMD from a non-human animal such as Danio rerio, Drosophila melanogaster, Xenopus laevis, or Gallus.

In some embodiments, the amino acid substitution(s) within the TMD includes one or more substitutions within a “GV” motif of the TMD. In some embodiments, at least one of such substitution(s) comprises a substitution to alanine. For example, one, two, three, four, five, or more of the amino acid residues of the sequence FMYVAAAAFVLLFFVGCGVLLS (SEQ ID NO: 4) may be substituted by a different amino acid residue. In some embodiments, the amino acid residue at position 18 and/or 19 of the “GV” motif within SEQ ID NO: 4 is substituted by a different amino acid residue. In some embodiments, the glycine residue at position 18 of SEQ ID NO: 4 is substituted by a different amino acid residue. In some embodiments, the valine residue at position 19 of SEQ ID NO: 4 is substituted by a different amino acid residue. In some embodiments, the transmembrane domain comprises an amino acid sequence having a sequence corresponding to SEQ ID NO: 4 with a mutation at the position corresponding to position 18 of SEQ ID NO: 4, such as G18A mutations. In some embodiments, the transmembrane domain comprises an amino acid sequence having a sequence corresponding to SEQ ID NO: 4 with a mutation at the position corresponding to position 19 of SEQ ID NO: 4, such as V19A mutations. The TMD can be derived from but longer or shorter than SEQ ID NO: 4. For instance, the TMD can be one, two, three, four, or more amino acids longer or shorter than SEQ ID NO: 4. In some embodiments, the TMD includes a sequence having at least 80% sequence identity, such as, at least 80%, at least 85%, at least 90%, at least 95%, at least 96%, at least 97%, at least 98%, or 99% sequence identity to SEQ ID NO: 4.

### Stop-Transfer Sequence (STS)

The chimeric receptors of the disclosure include an STS which comprises a charged, hydrophilic domain located between the TMD and the ICD. Without being bound to any particular theory, this domain disposed between the TMD and the ICD prevents the ICD from entering the plasma membrane. In some embodiments, a single-chain peptide comprising about 1 to about 40 amino acid residues (e.g., 1, 2, 3, 4, 5, 6, 7, 8, 9, 10, 11, 12, 13, 14, 15, 16, 17, 18, 19, 20, or more amino acid residues) in which most of the residues have charged side chains under physiological conditions can be used as a STS. In short STS embodiments (e.g., less than about 6 amino acids), about 5 or 6 of the amino acids will have charged side chains. In some embodiments, the STS includes about 1 to 15, about 5 to 20, about 8 to 25, about 10 to 30, about 12 to 35, about 14 to 40, about 5 to 40, about 10 to 35, about 15 to 30, about 20 to 25, about 20 to 40, about 10 to 30, about 4 to 20, or about 5 to 25 amino acid residues. In some embodiments, the STS includes about 4 to 10, about 5 to 12, about 6 to 14, about 7 to 18, about 8 to 20, about 9 to 22, about 10 to 24, or about 11 to 26 amino acid residues. In some embodiments, the STS includes about 4 to 10 residues, such as, 4, 5, 6, 7, 8, 9, or 10 amino acid residues.

In some embodiments, the STS includes a sequence having at least about 80% sequence identity, such as, at least 80%, at least 85%, at least 90%, at least 95%, at least 96%, at least 97%, at least 98%, or 99% sequence identity to the STS domain of a Type 1 receptor. In some embodiments, the STS includes an amino acid sequence having at least 90% sequence identity to the STS domain of a Type 1 receptor. In some embodiments, the STS includes a sequence having at least 70% sequence identity, such as, at least 70%, at least 75%, at least 80%, at least 85%, at least 90%, at least 95%, at least 96%, at least 97%, at least 98%, or 99% sequence identity to a STS sequence from Notch1, Notch2, Notch3, Notch4, CSF1R, CXCL16, DAG1, GHR, PTPRF, AGER, KL, NRG1, LRP1B, Jag2, EPCAM, KCNE3, CDH2, NRG2, PTPRK, BTC, EPHA3, IL1R2, or PTPRM. In some embodiments, the STS includes a sequence comprising only Lys (K) or Arg (R) in the first 4 residues. In some embodiments, the STS includes one, two, three, four, five, or more basic residues. In some embodiments, the STS includes five, four, three, two, one, or zero aromatic residues or residues with hydrophobic and/or bulky side chains.

In some embodiments, the STS includes a sequence having at least 80% sequence identity, such as, at least 80%, at least 85%, at least 90%, at least 95%, at least 96%, at least 97%, at least 98%, or 99% sequence identity to KRKRKH (SEQ ID NO: 5). In some embodiments, the STS includes an amino acid sequence having at least 90% sequence identity to SEQ ID NO: 5, and 75-78. The STS can be derived from but longer or shorter than SEQ ID NO: 5, and 75-78. For instance, the STS can be one, two, three, four, or more amino acids longer or shorter than SEQ ID NO: 5, and 75-78. In some embodiments, the STS includes a sequence having at least 80% sequence identity, such as, at least 80%, at least 85%, at least 90%, at least 95%, at least 96%, at least 97%, at least 98%, or 99% sequence identity to SEQ ID NO: 5, and 75-78. In some embodiments, the STS includes an amino acid sequence having at least 95% sequence identity to SEQ ID NO: 5, and 75-78. In some embodiments, the STS includes an amino acid sequence having at least 100% sequence identity to SEQ ID NO: 5, and 75-78. In some embodiments, the STS includes the amino acid sequence of SEQ ID NO: 5, and 75-78, wherein one, two, three, four, or five of the amino acid residues in SEQ ID NO: 5, and 75-78 is/are substituted by a different amino acid residue.

### Ram Sequence

The chimeric receptors of the disclosure may also include a RAM sequence which can be derived from human Notch 1. Without being bound to any particular theory, this domain disposed after the STS and can reduce basal signaling levels. In some embodiments, a single-chain peptide comprising about 1 to about 50 amino acid residues (e.g., 1, 2, 3, 4, 5, 6, 7, 8, 9, 10, 11, 12, 13, 14, 15, 16, 17, 18, 19, 20 or more amino acid residues) can be used as a RAM. In some embodiments, the RAM includes about 1 to 2, about 2 to 4, about 4 to 8, about 8 to 10, about 10 to 12, about 12 to 15 amino acid residues. In some embodiments, the RAM includes about 5 to 9 residues, such as, 5, 6, 7, 8, or 9 amino acid residues.

In some embodiments, the RAM includes a sequence having at least about 80% sequence identity, such as, at least 80%, at least 85%, at least 90%, at least 95%, at least 96%, at least 97%, at least 98%, or 99% sequence identity to the RAM domain of a human Notch 1 receptor. In some embodiments, the RAM includes an amino acid sequence having at least 90% sequence identity to the RAM domain of a human Notch 1. In some embodiments, the RAM includes a sequence having at least 70% sequence identity, such as, at least 70%, at least 75%, at least 80%, at least 85%, at least 90%, at least 95%, at least 96%, at least 97%, at least 98%, or 99% sequence identity to a STS sequence from Notch1, Notch2, Notch3, Notch4, CSF1R, CXCL16, DAG1, GHR, PTPRF, AGER, KL, NRG1, LRP1B, Jag2, EPCAM, KCNE3, CDH2, NRG2, PTPRK, BTC, EPHA3, IL1R2, or PTPRM.

In some embodiments, the RAM includes a sequence having at least 80% sequence identity, such as, at least 80%, at least 85%, at least 90%, at least 95%, at least 96%, at least 97%, at least 98%, or 99% sequence identity to SEQ ID Nos: 80-90. In some embodiments, the RAM includes an amino acid sequence having at least 90% sequence identity to SEQ ID Nos: 80-90. The RAM can be derived from but longer or shorter than SEQ ID Nos: 80-90. For instance, the RAM can be one, two, three, four, or more amino acids longer or shorter than SEQ ID NO: 5. In some embodiments, the RAM includes a sequence having at least 80% sequence identity, such as, at least 80%, at least 85%, at least 90%, at least 95%, at least 96%, at least 97%, at least 98%, or 99% sequence identity to SEQ ID Nos: 80-90. In some embodiments, the RAM includes an amino acid sequence having at least 95% sequence identity to SEQ ID Nos: 80-90. In some embodiments, the RAM includes an amino acid sequence having at least 100% sequence identity to SEQ ID Nos: 80-90. In some embodiments, the RAM includes the amino acid sequence of SEQ ID Nos: 80-90, wherein one, two, three, four, or five of the amino acid residues in SEQ ID Nos: 80-90 is/are substituted by a different amino acid residue.

### Intracellular Domain (ICD)

The chimeric receptors of this disclosure can also include, among others, an intracellular domain (ICD) containing at least two transcriptional regulators. Any one of the transcriptional regulators can include a human or humanized sequence. In some embodiments, the ICD contains two transcriptional regulators and at least one regulator includes a human or humanized sequence. In some embodiments, the ICD contains two transcriptional regulators and both include a human or humanized sequence. In other embodiments, the ICD contains more than two transcriptional regulators and at least one includes a human or humanized sequence. In yet other embodiments, the ICD contains more than two transcriptional regulators and all of them include a human or humanized sequence. In some embodiments, the binding of the ligand to the extracellular ligand-binding domain induces cleavage at a ligand-inducible proteolytic cleavage site between either of the transcriptional regulators and the linking polypeptide. A transcriptional regulator is a biochemical element that acts to activate or repress the transcription of a promoter-driven DNA sequence. Transcriptional regulators suitable for the compositions and methods of the disclosure can be naturally-occurring transcriptional regulators or can be engineered, designed, or modified so as to provide desired and/or improved properties, e.g., modulating transcription.

In some embodiments, the ICD described herein contains a first transcriptional regulator. In some embodiments, the ICD described herein further contains a second transcriptional regulator. In some non-limiting embodiments, the second transcriptional regulator augments the capacity of the first transcriptional regulator to modulate cell transcription.

In some embodiments, the transcriptional regulators described herein directly regulate expression of one or more genes involved in differentiation of the cell. In some embodiments, the transcriptional regulators indirectly modulate expression of one or more genes involved in differentiation of the cell by modulating the expression of an additional transcription factor which in turn modulates expression of one or more genes involved in differentiation of the cell. It will be understood by a skilled artisan that a transcriptional regulator (such as either the first or the second transcriptional regulator described herein, or both) can be a transcriptional activator or a transcriptional repressor. In some embodiments, the transcriptional regulator (such as either the first or the second transcriptional regulator described herein, or both) is a transcriptional repressor. In some embodiments, the transcriptional regulator (such as either the first or the second transcriptional regulator described herein, or both) is a transcriptional activator (a.k.a. transcriptional effector).

In some exemplary embodiments, at least one transcriptional regulator of the chimeric receptors of the disclosure includes a transcription factor DNA binding domain (DBD). Exemplary DBDs may include Gal4, tetR, ZFHD1, Zif268, and HAP1. In some exemplary embodiments, at least one transcriptional regulator of the chimeric receptors of the disclosure further includes a transactivation domain (TAD). In some embodiments, at least one transcriptional regulator of the chimeric receptors of the disclosure includes a nuclear localization sequence.

In some embodiments, at least one transcriptional regulator of the chimeric receptors of the disclosure includes a zinc finger-containing transcriptional effector (ZTE) which includes one or more zinc finger proteins or zinc finger motifs (ZFs). For example, a transcriptional regulator of the chimeric receptors of the disclosure may include a DBD containing one or more ZFs. A ZF is a finger-shaped fold in a protein that permits it to interact with nucleic acid sequences such as DNA and RNA. Such finger-shaped fold is well known in the art. The fold is generally created by the binding of specific amino acids in the protein to a zinc atom, and is stabilized by the co-ordination of a zinc ion between four largely invariant (depending on zinc finger framework type) Cys and/or His residues. Exemplary zinc finger proteins may include ZF3, ZF6, ZF10, etc.

The term “motif” as used herein refers to a structural motif A ZF motif is a relatively small polypeptide domain having a supersecondary structure, and includes approximately 30 amino acids and folds to form an α-helix adjacent an antiparallel β-sheet (known as a ββα-fold), and is stabilized by a zinc ion. A ZF domain recognizes and binds to a nucleic acid triplet, or an overlapping quadruplet (as explained below), in a double-stranded DNA target sequence. Naturally-occurring zinc finger domains (also known as ZF proteins) have been well studied and described in the literature. Natural ZF proteins can regulate the expression of genes as well as nucleic acid recognition, reverse transcription and virus assembly. Additional information in this regard can be found in, for example, U.S. Pat. No. 10,138,493.

C2H2 zinc fingers (C2H2-ZFs) are among the most prevalent type of vertebrate DNA-binding domain, and generally appear in tandem arrays (ZFAs), with sequential C2H2-ZFs each contacting three (or more) sequential bases. C2H2-ZFs can be assembled in a modular fashion. Given a set of modules with defined three-base specificities, modular assembly also presents a way to construct artificial proteins with specific DNA-binding preferences.

ZF-containing proteins generally contain strings or chains of ZF motifs, forming an array of ZF (ZFA). Thus, a ZF protein may include two or more ZFs, e.g., a ZFA consisting of 2 or more ZF motifs, which may be directly adjacent one another (e.g., separated by a short linker sequence), or may be separated by longer, flexible or structured polypeptide sequences. For example, a ZFA can have six ZF motifs (a 6-finger ZFA), seven ZF motifs (a 7-finger ZFA), or eight ZF motifs (an 8-finger ZFA), arranged in tandem. Directly adjacent ZF domains are generally expected to bind to contiguous nucleic acid sequences, e.g., to adjacent trinucleotides/triplets. In some cases, cross-binding may also occur between adjacent ZF and their respective target triplets, which may help to strengthen or enhance the recognition of the target sequence, and leads to the binding of overlapping quadruplet sequences. By comparison, distant ZF domains within the same protein may recognize, and/or bind to, non-contiguous nucleic acid sequences or even to different molecules (e.g., protein rather than nucleic acid).

In some embodiments, the engineered Notch receptors of the disclosure include a zinc finger-containing transcriptional effector (ZTE) having a DNA binding zinc finger protein domain (ZF protein domain) and another domain through which the protein exerts its effect (effector domain). As described in further detail below, exemplary effector domains suitable for the engineered Notch receptors of the disclosure include, but are not limited to, transcriptional activating domains (e.g., TADs), epigenetic effector domains, and DNA modifying enzymes.

In some embodiments, the engineered Notch receptors of the disclosure include a transcriptional effector having a non-zinc finger DNA binding domain (e.g., DNA binding domains not having a zinc finger structure) and another domain through which the protein exerts its effect (effector domain). Exemplary non-zinc finger DNA binding domains include the domain derived from PAX6. As described in further detail below, exemplary effector domains suitable for the engineered Notch receptors of the disclosure include, but are not limited to, transcriptional activating domains (e.g., TADs), epigenetic effector domains, and DNA modifying enzymes. In some embodiments, the engineered Notch receptors of the disclosure include 1 to about 10 DNA binding domains, each of which independently includes a sequence having at least about 90% identity to the sequence of SEQ ID NO: 12 (PAX6). In some embodiments, the DNA binding domain of the transcriptional effector has at least about 90%, at least about 95%, at least about 96%, at least about 97%, at least about 98%, or at least about 99% sequence identity to the sequence of SEQ ID NO: 12 (PAX6).

In some embodiments, the engineered Notch receptors of the disclosure include a ZTE with two or more, e.g., 3 or more, for example, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more (e.g., up to approximately 30 or 32) ZF motifs arranged adjacent one another in tandem, forming arrays of ZF motifs or ZFA. In some embodiments, the ZTE includes at least 3 ZF motifs, at least 4 ZF motifs, at least 5 ZF motifs, or at least 6 ZF motifs, at least 7 ZF motifs, at least 8 ZF motifs, at least 9 ZF motifs, at least 10 ZF motifs, at least 11 or at least 12 ZF motifs; and in some cases at least 18 ZF motifs. In some embodiments, the ZTE of the engineered Notch receptors disclosed herein contains up to 6, 7, 8, 10, 11, 12, 16, 17, 18, 22, 23, 24, 28, 29, 30, 34, 35, 36, 40, 41, 42, 46, 47, 48, 54, 55, 56, 58, 59, or 60 ZF motifs. In some embodiments, the ZTE of the disclosure bind to orthogonal target nucleic acid binding sites. That is, the ZFs or ZFAs in ZF domain of the ZTE binds orthogonal target nucleic acid sequences. In some embodiments, the orthogonal target nucleic acid binding sites are contiguous. In some embodiments, the ZTE of the engineered Notch receptors disclosed herein binds target orthogonal specific DNA sequences and have, for example, reduced or minimal functional binding potential in a eukaryotic genome.

In some embodiments of the disclosure, the ZTE includes: (a) a first domain including a DNA-binding zinc finger protein domain (ZF protein domain), and (b) a second domain through which the ZTE exerts its effect (effector domain), wherein the ZTE has the following formula I: [effector domain]a-[ZF protein domain]-[effector domain]b (Formula I) wherein a and b are each independently an integer from 0 to 5, and at least one of a and b is not 0; wherein the ZF protein domain includes 1 to about 10 zinc finger arrays (ZFA); wherein the ZFA includes about 1 to 3, about 3 to 6, or about 6 to about 8 zinc finger motifs according to formula II (from N-terminal to C-terminal):

(Formula II)
XcCXdCXe-(helix)-HXfH-L2

wherein L2 is a linker peptide having about 4-6 amino acid residues, C is Cys, H is His, each X is independently any amino acid, c is an integer from 0 to 3, d is an integer from 1 to 5, e is an integer from 2 to 7, f is an integer from 3 to 6, and (helix) is a peptide domain of about 6 amino acids that forms an α-helix, wherein the ZFA is capable of binding a specific nucleic acid sequence.

In some embodiments, the ZF protein domain of the engineered Notch receptors disclosed herein includes 1 to about 10 ZFA, each of which independently includes a sequence having at least about 90% identity to a sequence selected from the group consisting of SEQ ID NOs: 6-11. In some embodiments, the ZFA includes a sequence having at least about 90%, at least about 95%, at least about 96%, at least about 97%, at least about 98%, or at least about 99% sequence identity to a sequence selected from the group consisting of SEQ ID NOs: 6-11. In some embodiments, the ZFA sequence has at least about 90%, at least about 95%, at least about 96%, at least about 97%, at least about 98%, or at least about 99% sequence identity to the sequence of SEQ ID NO: 6 (ZF3). In some embodiments, the ZFA sequence has at least about 90%, at least about 95%, at least about 96%, at least about 97%, at least about 98%, or at least about 99% sequence identity to the sequence of SEQ ID NO: 7 (ZF6). In some embodiments, the ZFA sequence has at least about 90%, at least about 95%, at least about 96%, at least about 97%, at least about 98%, or at least about 99% sequence identity to the sequence of SEQ ID NO: 8 (ZF6/SV40NLS). In some embodiments, the ZFA sequence has at least about 90%, at least about 95%, at least about 96%, at least about 97%, at least about 98%, or at least about 99% sequence identity to the sequence of SEQ ID NO: 9 (ZF6/Notch1NLS). In some embodiments, the ZFA sequence has at least about 90%, at least about 95%, at least about 96%, at least about 97%, at least about 98%, or at least about 99% sequence identity to the sequence of SEQ ID NO: 10 (ZF10/SV40NLS). In some embodiments, the ZFA sequence has at least about 90%, at least about 95%, at least about 96%, at least about 97%, at least about 98%, or at least about 99% sequence identity to the sequence of SEQ ID NO: 11 (ZF10/Notch1NLS). In some embodiments, the ZFA sequence has at least about 90%, at least about 95%, at least about 96%, at least about 97%, at least about 98%, or at least about 99% sequence identity to the sequence of ZF2, ZF4, or ZF11.

In some embodiments, the ZF protein domain of the engineered Notch receptors disclosed herein includes 1 to about 10 ZFA, each of which independently includes a sequence having about 100% sequence identity to a sequence selected from the group consisting of SEQ ID NOs: 6-11. In some embodiments, the ZFA sequence has about 100% sequence identity to the sequence of SEQ ID NO: 6 (ZF3). In some embodiments, the ZFA sequence has about 100% sequence identity to the sequence of SEQ ID NO: 7 (ZF6). In some embodiments, the ZFA sequence has about 100% sequence identity to the sequence of SEQ ID NO: 8 (ZF6/SV40NLS). In some embodiments, the ZFA sequence has about 100% sequence identity to the sequence of SEQ ID NO: 9 (ZF6/Notch1NLS). In some embodiments, the ZFA sequence has about 100% sequence identity to the sequence of SEQ ID NO: 10 (ZF10/SV40NLS). In some embodiments, the ZFA sequence has about 100% sequence identity to the sequence of SEQ ID NO: 11 (ZF10/Notch1NLS). In some embodiments, the ZFA sequence has about 100% sequence identity to the sequence of SEQ ID NO: 11 (ZF10/Notch1NLS). In some embodiments, the ZFA sequence has about 100% sequence identity to the sequence of ZF2, ZF4, or ZF11.

In some embodiments, the ZF protein domain includes multiple ZFAs having the same amino acid sequences. In some embodiments, the ZF protein domain includes multiple ZFAs whose amino acid sequences are different from one another.

In some embodiments, the ZF protein domain of the engineered Notch receptors disclosed herein includes one or more ZFAs that are independently capable of specifically binding to a target nucleic acid sequence selected from the group consisting of SEQ ID NOs: 6-11. In some embodiments, at least one ZFA is capable of specifically binding to a target nucleic acid sequence having the sequence of SEQ ID NO: 6. In some embodiments, at least one ZFA is capable of specifically binding to a target nucleic acid sequence having the sequence of SEQ ID NO: 7. In some embodiments, at least one ZFA is capable of specifically binding to a target nucleic acid sequence having the sequence of SEQ ID NO: 8. In some embodiments, at least one ZFA is capable of specifically binding to a target nucleic acid sequence having the sequence of SEQ ID NO: 9. In some embodiments, at least one ZFA is capable of specifically binding to a target nucleic acid sequence having the sequence of SEQ ID NO: 10. In some embodiments, at least one ZFA is capable of specifically binding to a target nucleic acid sequence having the sequence of SEQ ID NO: 11. In some embodiments, the ZF protein domain of the engineered Notch receptors disclosed herein includes one or more ZFAs that are independently capable of specifically binding to ZF2, ZF4, or ZF11.

As described herein, the zinc finger-containing transcriptional effector (ZTE) of the engineered Notch receptors disclosed herein includes a second domain through which the ZTE exerts its effect (effector domain). Exemplary effector domains suitable for the engineered Notch receptors of the disclosure include, but are not limited to, transcriptional activating domains (e.g., TADs), epigenetic effector domains, and DNA modifying enzymes. Non-limiting examples of transcription-activating domains (TADs) suitable for use in the compositions and methods disclosed herein include herpes simplex virus protein 16 (HSV VP16) activation domain; an activation domain consisting of four tandem copies of VP16 (VP64); a p65 activation domain of NFκB; an Epstein-Barr virus R transactivator activation domain (Rta); a tripartite activator consisting of VP64, and Rta activation domains (VPR); and a histone acetyltransferase core domain of the human E1A-associated protein p300 (p300 HAT core activation domain). In some embodiments, the effector domain of the ZTE includes a p65 activation domain of NFκB.

In some embodiments, at least one transcriptional regulator of the chimeric receptors of the disclosure is selected from Gal4-VP16, Gal4-VP64, tetR-VP64, ZFHD1-VP64, Gal4-KRAB, and HAP1-VP16. In some embodiments, the transcriptional regulator (either the first or the second transcriptional regulator described herein, or both) is Gal4-VP64. In some embodiments, the transcriptional regulator (either the first or the second transcriptional regulator described herein, or both) has a sequence derived from or selected from the group consisting of VP64, p65, KRAB transactivating variants, and VP16. In some exemplary embodiments, the transcriptional regulator (such as either the first or the second transcriptional regulator described herein, or both) has a sequence derived from a TAD of human or humanized p65. In some exemplary embodiments, the transcriptional regulator (such as either the first or the second transcriptional regulator described herein, or both) has a sequence derived from a human or humanized p65. In other exemplary embodiments, the transcriptional regulator (either the first or the second transcriptional regulator described herein, or both) has a sequence derived from a TAD of human or humanized HSF-1, GATA3, HIF1a, GR Tau1, ATF6, ELF3, p53, MIER3, MLXIPL, NFE2L1, or PTF1A. In other exemplary embodiments, the transcriptional regulator (either the first or the second transcriptional regulator described herein, or both) has a sequence derived from human or humanized HSF-1, GATA3, HIF1a, GR Tau1, ATF6, ELF3, p53, MIER3, MLXIPL, NFE2L1, or PTF1A.

In some embodiments, the human or humanized sequence contains a linear amino acid sequence motif. In some embodiments, the first and the second transcriptional regulators described herein is directly fused together, with or without a linker sequence.

In some embodiments, the transcriptional regulator (either the first or the second transcriptional regulator described herein, or both) includes a sequence having at least 80% sequence identity, such as, at least 80%, at least 85%, at least 90%, at least 95%, at least 96%, at least 97%, at least 98%, or 99% sequence identity to one or more of SEQ ID NOs: 15-22. In some embodiments, the transcriptional regulator (either the first or the second transcriptional regulator described herein, or both) includes an amino acid sequence having at least 90% sequence identity to one or more of SEQ ID NOs: 15-22. In some embodiments, the transcriptional regulator (either the first or the second transcriptional regulator described herein, or both) includes an amino acid sequence having at least 95% sequence identity to one or more of SEQ ID NOs: 15-22. In some embodiments, the transcriptional regulator (either the first or the second transcriptional regulator described herein, or both) includes an amino acid sequence having at least 100% sequence identity to one or more of SEQ ID NOs: 15-22. In some embodiments, the transcriptional regulator (either the first or the second transcriptional regulator described herein, or both) includes an amino acid sequence of one or more of SEQ ID NOs: 15-22, wherein one, two, three, four, or five of the amino acid residues in one or more of SEQ ID NOs: 15-22 is/are substituted by a different amino acid residue.

In some embodiments, the transcriptional regulator (either the first or the second transcriptional regulator described herein, or both) includes a sequence having at least 80% sequence identity, such as, at least 80%, at least 85%, at least 90%, at least 95%, at least 96%, at least 97%, at least 98%, or 99% sequence identity to one or more of SEQ ID NOs: 24-34 and 95-98. In some embodiments, the transcriptional regulator (either the first or the second transcriptional regulator described herein, or both) includes an amino acid sequence having at least 90% sequence identity to one or more of SEQ ID NOs: 24-34 and 95-98. In some embodiments, the transcriptional regulator (either the first or the second transcriptional regulator described herein, or both) includes an amino acid sequence having at least 95% sequence identity to one or more of SEQ ID NOs: 24-34 and 95-98. In some embodiments, the transcriptional regulator (either the first or the second transcriptional regulator described herein, or both) includes an amino acid sequence having at least 100% sequence identity to one or more of SEQ ID NOs: 24-34 and 95-98. In some embodiments, the transcriptional regulator (either the first or the second transcriptional regulator described herein, or both) includes an amino acid sequence of one or more of SEQ ID NOs: 24-34 and 95-98, wherein one, two, three, four, or five of the amino acid residues in one or more of SEQ ID NOs: 24-34 is/are substituted by a different amino acid residue.

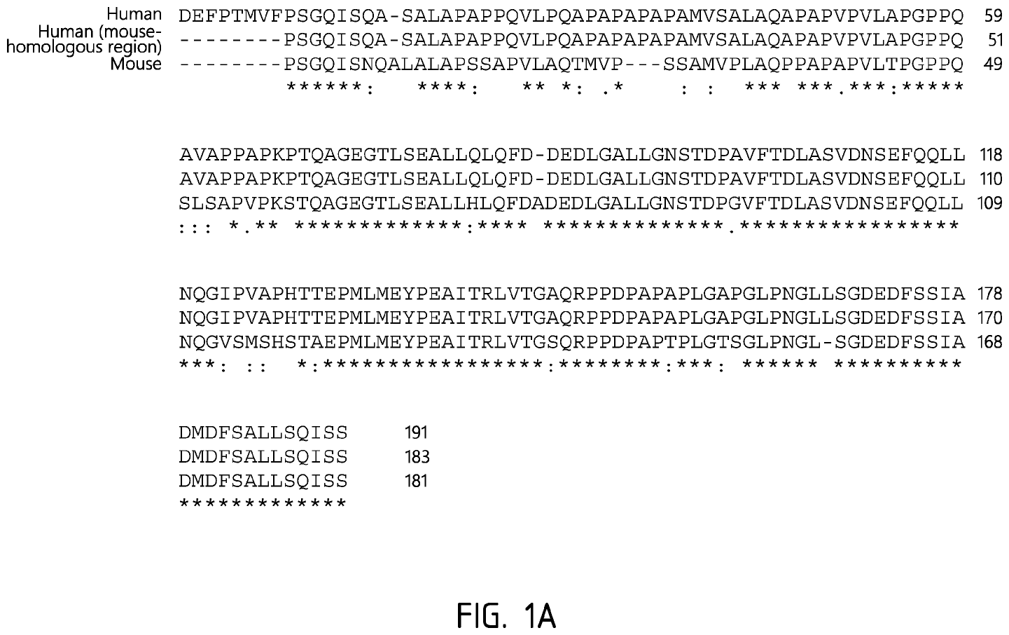

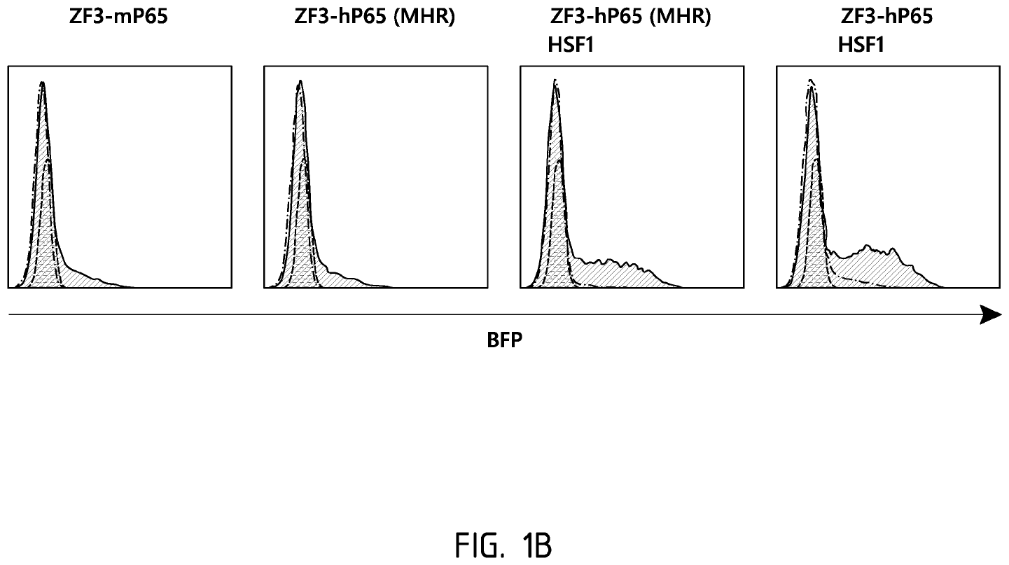

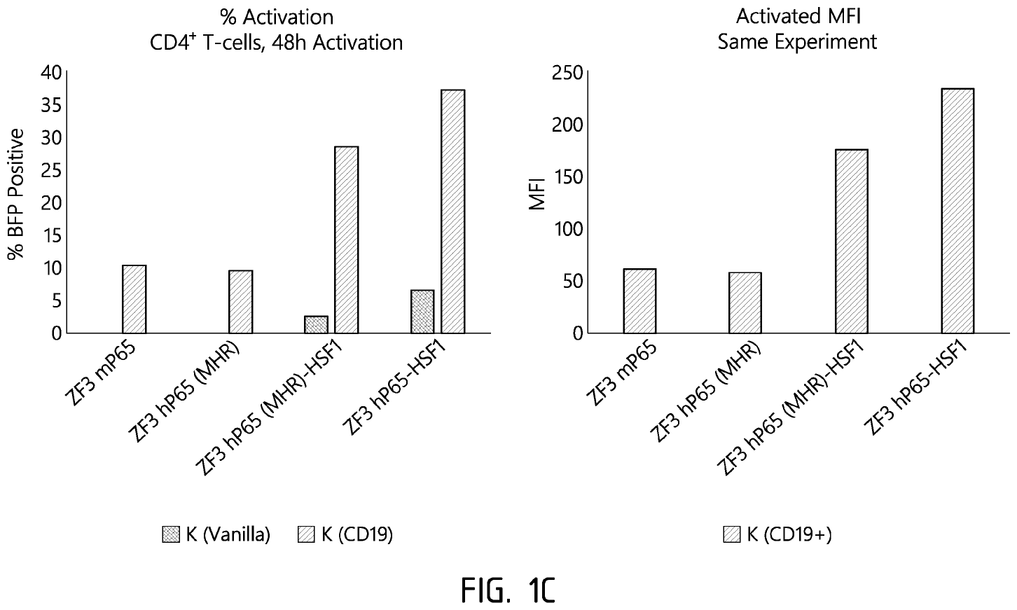

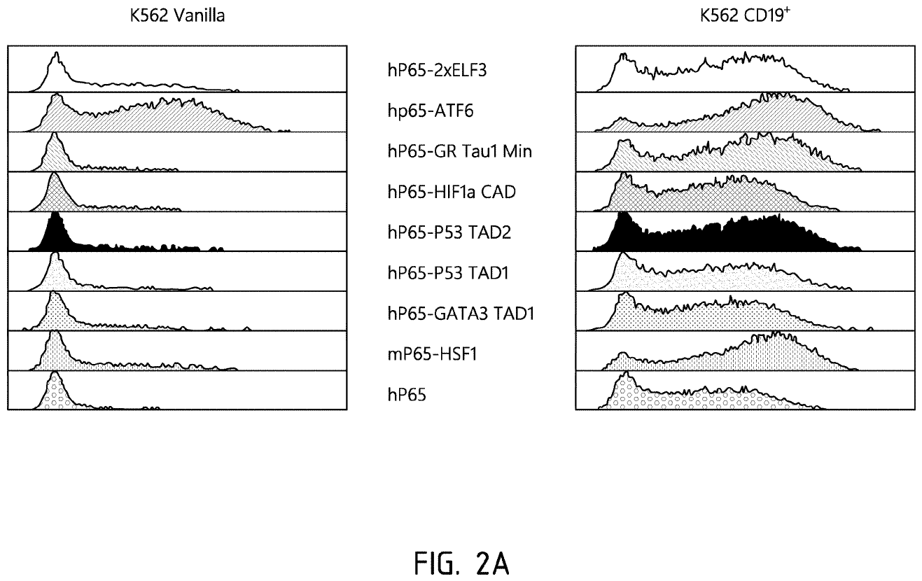

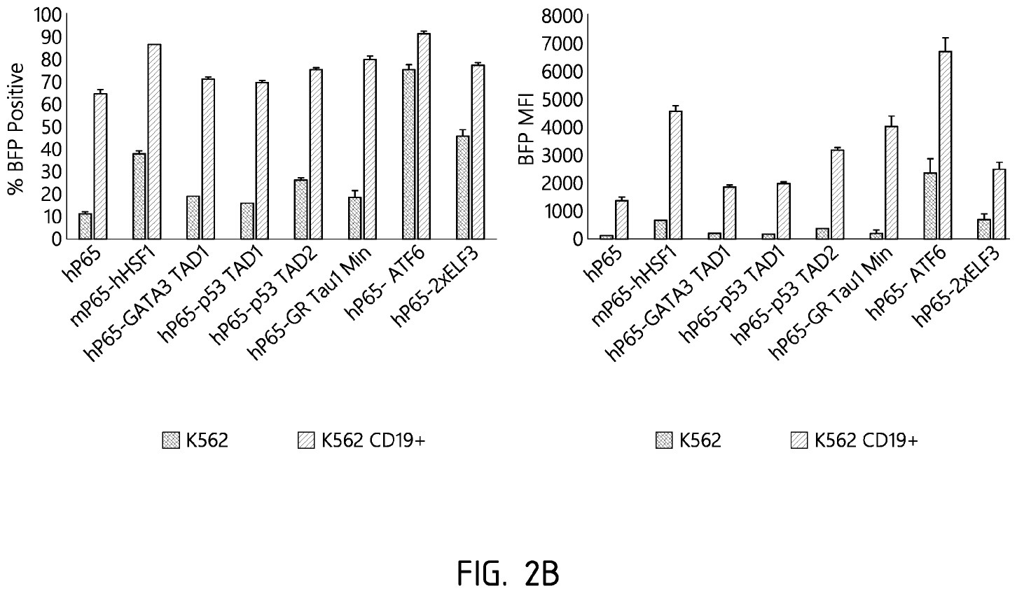

In some embodiments, the transcriptional regulators of the chimeric receptors of the disclosure include ZF3, a TAD derived from a TAD of a human or humanized p65, and a TAD derived from a TAD of a human or humanized HSF1. In some embodiments, the transcriptional regulators of the chimeric receptors of the disclosure include ZF3, a human or humanized p65, and a human or humanized HSF1. In other exemplary embodiments, the transcriptional regulators of the chimeric receptors of the disclosure include ZF3, a human or humanized p65, and a human or humanized GR Tau1. In some exemplary embodiments, the transcriptional regulators of the chimeric receptors of the disclosure include ZF3, a human or humanized p65, and a human or humanized p53. In other exemplary embodiments, the transcriptional regulators of the chimeric receptors of the disclosure include ZF3, a human or humanized p65, and a human or humanized ATF6. As demonstrated in the Examples and Figures, the addition of the human or humanized sequences significantly improves the transcriptional regulation activity of the chimeric receptors. See, for example, FIGS. 1B-1C and FIGS. 2A-2B.

The chimeric receptors of the disclosure includes an intracellular domain (ICD) comprising, in any order: (i) an intracellular signaling domain (SD) derived from a signaling molecule; (ii) an activation domain; and (iii) two or more transcriptional regulators (e.g., transcriptional effectors). In other words, the ICD of the chimeric receptors of the disclosure can have at least three distinct domains. The three distinct domains can be arranged in any order, and can be linked by a linker. In some embodiments, the three distinct domains are linked via (GS)n linkers. The n can be any number selected from 1 to 100. For example, the n can be 2, 3, 4, 5, 6, 7, 8, 9, or 10. In other embodiments, the n can be 11, 12, 13, 14, 15, 16, 17, 18, 19, or 20. An exemplary GS linker can have 3 GS repeats and the sequence of GSGSGSGS (SEQ ID NO: 72). Other exemplary linkers may include, GSTCR (SEQ ID NO: 13), M (SEQ ID NO: 14), SGQGGGGS (SEQ ID NO: 23), etc. Exemplary chimeric receptors with or without various linkers are shown in Tables 1 and 2 and the Sequence Table after Examples. One skilled in the art would know how to modify the linker to suit specific uses.

In some embodiments, the intracellular domain of the chimeric receptors of the disclosure further comprises an intracellular signaling domain (SD). Exemplary SDs include 4-1BB, BAFF-R, BCMA, BTLA, CD2, CD200R, CD244, CD28, CD300a, CD300f, CD40, CD7, CD72, CD96, CRACC, CRTAM, CTLA4, CXADR, DC-SIGN, GITR, HAVCR2, ICOS, ILT2, ILT3, ILT4, KIR2DL1, KIR3DL1, KLRG1, LAG3, LAIR1, NKG2D, NKR-P1A, NTB-A, PD1, Siglec-3, TACI, TIGIT, TLT-1, and TNR8 (CD30). In some embodiments, the SD in the chimeric receptors of the present disclosure is selected from the group consisting of: OX40, ICOS, 4-1BB, CTLA4, CD28, CD30, CD2, CD27, and CD226. In some embodiments, the SD in the chimeric receptors of the present disclosure is a biologically active fragment, mutant, variant, or ortholog of any one of the exemplary SDs disclosed herein.

In some embodiments, the chimeric receptors of the disclosure includes a costimulatory domain comprises a sequence that is derived from a signaling molecule. The signaling molecule can be a protein selected from a class 1 or a class 3 human membrane protein. In some embodiments, the signaling molecule is selected from CD28, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, 4-1BB, OX40, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-P1A, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC, and derivatives, mutants, variants, fragments and combinations thereof. In other embodiments, the signaling molecule is selected from the list consisting of OX40, ICOS, 4-1BB, CTLA4, CD28, CD30, CD2, CD27, and CD226, and derivatives, mutants, variants, fragments and combinations thereof. In some embodiments, the signaling molecule is selected from the group consisting of 4-1BB, BAFF-R, BCMA, BTLA, CD2, CD200R, CD244, CD28, CD300a, CD300f, CD40, CD7, CD72, CD96, CRACC, CRTAM, CTLA4, CXADR, DC-SIGN, GITR, HAVCR2, ICOS, ILT2, ILT3, ILT4, KIR2DL1, KIR3DL1, KLRG1, LAG3, LAIR1, NKG2D, NKR-P1A, NTB-A, PD1, Siglec-3, TACI, TIGIT, TLT-1, and TNR8 (CD30), and derivatives, mutants, variants, fragments and combinations thereof. In other embodiments, the signaling molecule is CD28 or 4-1BB. In one exemplary embodiment, the costimulatory domain comprises a sequence that is derived from CD28. In another exemplary embodiment, the costimulatory domain comprises a sequence that is derived from 4-1BB.

In some embodiments, the activation domain includes one or more conserved amino acid motifs that serve as substrates for phosphorylation such as, for example, immunoreceptor tyrosine-based activation motifs (ITAMs). In some embodiments, the activation domain includes at least 1, at least 2, at least 3, at least 4, or at least 5 specific tyrosine-based motifs selected from ITAM motifs, an ITIM motifs, or related intracellular motifs that serve as a substrate for phosphorylation. In some embodiments of the disclosure, the activation domain of the intracellular signaling domain includes at least 1, at least 2, at least 3, at least 4, or at least 5 ITAMs. Generally, any activation domain including an ITAM can be suitably used for the construction of the chimeric receptor s as described herein. An ITAM generally includes a conserved protein motif that is often present in the tail portion of signaling molecules expressed in many immune cells. The motif may include two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways

In some embodiments, the activation domain is derived from CD3ζ, CD3σ, CD3γ, and CD3ε. For instance, in some embodiments, the ITAMs are derived from CD3ζ, CD3σ, CD3γ, and CD3ε. In one exemplary embodiment, the ITAM is derived from CD3ζ. In certain embodiments, the ITAM comprises a sequence that is at least about 80, 85, 90, 95, 96, 97, 98, 99, or 100% identical to a CD3ζ ITAM. In some embodiments, the activation domain comprises at least 1, at least 2, at least 3, at least 4, or at least 5 ITAMs independently selected from the ITAMs derived from CD3ζFcRγ, and combinations thereof. In some embodiments, the activation domain comprises a CD3ζ ITAM.

In some embodiments, at least one transcriptional regulator of the chimeric receptors of the disclosure includes a transcriptional repressor domain. Non-limiting examples of transcriptional repressor domains suitable for use in the compositions and methods disclosed herein include a Kruppel associated box repression domain (KRAB); a Repressor Element Silencing Transcription Factor repression domain (REST); a WRPW motif of the hairy-related basic helix-loop-helix repressor proteins repression domain (WRPW); a DNA (cytosine-5)-methyltransferase 3B repression domain (DNMT3B); and an HP1 alpha chromoshadow repression domain. In some embodiments, the transcriptional repressor domain includes a KRAB repressor domain.

Accordingly, at least one transcriptional regulator of the chimeric receptors of the disclosure includes an epigenetic effector domain. Examples of epigenetic effector domain suitable for use in the compositions and methods disclosed herein include, but are not limited to, DNA methyltransferases DNMT (DNMT1, DNMT3), HAT1, GCN5, PCAF, MLL, SET, DOT1, SUV39H, G9a, KAT2A/B, EZH1/2, TET1/2, SIRT family protein effector domains, histone deacetylases, LSD1, and KDM family protein effector domains.

Effectors domains suitable for the compositions and methods of the disclosure can be naturally-occurring transcriptional regulators or can be engineered, designed, or modified so as to provide desired and/or improved properties, e.g., modulating transcription a eukaryotic cell. In some embodiments, the effector domain is derived from an animal protein. In some embodiments, the effector domain is derived from a mammalian protein. In some embodiments, the effector domain is derived from non-human primate protein. In some embodiments, the effector domain is derived from a human protein.

In some embodiments, the ICD of the chimeric receptors disclosed herein further includes a nuclear transport signal sequence (NLS). Nuclear localization signals (NLSs) are short peptide motifs that mediate the nuclear import of proteins by binding to their receptors, known as importins (karyopherins). Exemplary NLS may be derived from SV40 or Notch 1 NLS sequences.

It will be understood by one having ordinary skill in the art that a transcriptional effector can be a transcriptional activator or a transcriptional repressor. In some embodiments, the transcriptional effector is a transcriptional repressor. In some embodiments, the transcriptional effector is a transcriptional activator. In some embodiments, the transcriptional effector directly regulates differentiation of the cell. In some embodiments, the transcriptional effector indirectly modulates (e.g., regulates) differentiation of the cell by modulating the expression of a second transcription factor.

### Additional Domains

In some embodiments, the chimeric receptors provided herein (e.g., the extracellular domains located N-terminally to the TMD) can further include an additional region. In some embodiments, the chimeric receptors can include a membrane localization signal such as a CD8A signal. In other embodiments, the chimeric receptors can include a detectable label, such as a myc tag or His tag, and the like. In additional embodiments, the chimeric receptors provided herein can also include a tumor-specific cleavage site, or a disease-specific cleavage site. In further embodiments, the chimeric receptors provided herein can include a combination of these additional regions.

In some embodiments, the chimeric receptors of the disclosure include: (a) a linking polypeptide including an amino acid sequence having at least 80% sequence identity to SEQ ID NO: 3; (b) a transmembrane domain including an amino acid sequence having at least 80% sequence identity to SEQ ID NO: 4; and (c) a stop transfer sequence domain including an amino acid sequence having at least 80% sequence identity to SEQ ID NO: 5.

In some embodiments, the chimeric receptors of the disclosure include: (a) an extracellular ligand-binding domain having at least 80% sequence identity to any one of SEQ ID NOs: 1 and 2; (b) a linking polypeptide including an amino acid sequence having at least 80% sequence identity to SEQ ID NO: 3; (c) a transmembrane domain including an amino acid sequence having at least 80% sequence identity to SEQ ID NO: 4; (d) a stop transfer sequence domain including an amino acid sequence having at least 80% sequence identity to SEQ ID NO: 5; and (e) an intracellular domain including one or more amino acid sequences having at least 80% sequence identity to one or more of SEQ ID NOs: 6-12.

In some embodiments, the chimeric receptors of the disclosure include: (a) an extracellular ligand-binding domain having at least 80% sequence identity to any one of SEQ ID NOs: 1 and 2; (b) a linking polypeptide including an amino acid sequence having at least 80% sequence identity to SEQ ID NO: 3; (c) a transmembrane domain including an amino acid sequence having at least 80% sequence identity to SEQ ID NO: 4; (d) a stop transfer sequence domain including an amino acid sequence having at least 80% sequence identity to SEQ ID NO: 5; and (e) an intracellular domain including at least two amino acid sequences having at least 80% sequence identity to one or more of SEQ ID NOs: 6-12 and SEQ ID NOs: 15-22, respectively.

In some embodiments, the chimeric receptors of the disclosure include: (a) an extracellular ligand-binding domain having at least 80% sequence identity to any one of SEQ ID NOs: 1 and 2; (b) a linking polypeptide including an amino acid sequence having at least 80% sequence identity to SEQ ID NO: 3; (c) a transmembrane domain including an amino acid sequence having at least 80% sequence identity to SEQ ID NO: 4; (d) a stop transfer sequence domain including an amino acid sequence having at least 80% sequence identity to SEQ ID NO: 5; and (e) an intracellular domain including three amino acid sequences, each having at least 80% sequence identity to any one of SEQ ID NOs: 6-12, SEQ ID NOs: 15-22, and SEQ ID NOs: 24-34, linked directly with or without a linker.

In some exemplary embodiments, the chimeric receptors of the disclosure includes: (a) an extracellular ligand-binding domain having a sequence set forth in SEQ ID NO: 1 or 2; (b) a linking polypeptide including an amino acid sequence having a sequence set forth in SEQ ID NO: 3; (c) a transmembrane domain including an amino acid sequence having a sequence set forth in SEQ ID NO: 4; (d) a stop transfer sequence domain including an amino acid sequence having a sequence set forth in SEQ ID NO: 5; and (e) an intracellular domain including three amino acid sequences, each having a sequence set forth in SEQ ID NOs: 6-12, SEQ ID NOs: 15-22, and SEQ ID NOs: 24-34, linked directly or by a linker.

In some embodiments, the chimeric receptors of the disclosure include: (a) an extracellular ligand-binding domain having at least 80% sequence identity to any one of SEQ ID NOs: 1 and 2; (b) a linking polypeptide including an amino acid sequence having at least 80% sequence identity to SEQ ID NO: 3; (c) a transmembrane domain including an amino acid sequence having at least 80% sequence identity to SEQ ID NO: 4; and (d) an intracellular domain including one or more amino acid sequences having at least 80% sequence identity to one or more of SEQ ID NOs: 6-12, 15-22, and 24-34.

In some embodiments, the chimeric receptors of the disclosure include: (a) an extracellular ligand-binding domain having at least 80% sequence identity to any one of SEQ ID NOs: 1 and 2; (b) a linking polypeptide including an amino acid sequence having at least 80% sequence identity to SEQ ID NO: 3; (c) a transmembrane domain including an amino acid sequence having at least 80% sequence identity to SEQ ID NO: 4; and (d) an intracellular domain including one or more amino acid sequences having at least 80% sequence identity to one or more of SEQ ID NOs: 6-12, 15-22, 24-34, 93-98.

In some embodiments, the chimeric receptor of the disclosure includes an amino acid sequence having at least about 80%, 90%, 95%, 96%, 97, 98%, 99%, or 100% sequence identity to a chimeric receptor disclosed herein. In some embodiments, provided herein are chimeric receptors including an amino acid sequence having at least about 80%, 90%, 95%, 96%, 97, 98%, 99%, or 100% sequence identity to any one of SEQ ID NOs: 35-71 and 99-123.

In some embodiments, the various components of the chimeric receptors of this disclosure are linked via (GS)n linkers. The n can be any number selected from 1 to 100. For example, the n can be 2, 3, 4, 5, 6, 7, 8, 9, or 10. In other embodiments, the n can be 11, 12, 13, 14, 15, 16, 17, 18, 19, or 20. An exemplary GS linker can have 3 GS repeats and the sequence of GSGSGSGS (SEQ ID NO: 72). Other exemplary linkers may include, GSTCR (SEQ ID NO: 13), M (SEQ ID NO: 14), SGQGGGGS (SEQ ID NO: 23), etc. Exemplary chimeric receptors with or without various linkers are shown in Tables 1 and 2 and the Sequence Table after Examples. One skilled in the art would know how to modify the linker to suit specific uses.

### Nucleic Acid Molecules

In another aspect, provided herein are various nucleic acid molecules including nucleotide sequences encoding the chimeric receptors of the disclosure, including expression cassettes, and expression vectors containing these nucleic acid molecules operably linked to heterologous nucleic acid sequences such as, for example, regulatory sequences which allow in vivo expression of the receptor in a host cell.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, provided herein is a nucleic acid molecule including a nucleotide sequence encoding a chimeric receptor including, from N-terminus to C-terminus: (a) an extracellular ligand-binding domain having a binding affinity for a selected ligand; (b) a linking sequence; (c) a transmembrane domain including one or more ligand-inducible proteolytic cleavage sites; and (d) an intracellular domain including (i) an intracellular signaling domain (SD) comprising at least one costimulatory domain derived from a signaling molecule and an activation domain, and (ii) a transcriptional regulator, wherein binding of the selected ligand to the extracellular ligand-binding domain induces cleavage at a ligand-inducible proteolytic cleavage site disposed between the transcriptional regulator and the hinge domain.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the chimeric receptor as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, provided herein are nucleic acid molecules encoding a polypeptide with an amino acid sequence having at least about 80%, 90%, 95%, 96%, 97, 98%, 99%, or 100% sequence identity to a chimeric receptor disclosed herein. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOs: 1-72. In some embodiments, the nucleic acid molecules encode a polypeptide with an amino acid sequence having at least about 80%, 90%, 95%, 96%, 97, 98%, 99%, or 100% sequence identity to any one of SEQ ID NOs: 35-71 and 99-123. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOs: 1 and 2. In some embodiments, provided herein are nucleic acid molecules encoding a polypeptide with an amino acid sequence having at least about 80%, 90%, 95%, 96%, 97, 98%, 99%, or 100% sequence identity to SEQ ID NO: 3. In some embodiments, provided herein are nucleic acid molecules encoding a polypeptide with an amino acid sequence having at least about 80%, 90%, 95%, 96%, 97, 98%, 99%, or 100% sequence identity to SEQ ID NO: 4. In some embodiments, provided herein are nucleic acid molecules encoding a polypeptide with an amino acid sequence having at least about 80%, 90%, 95%, 96%, 97, 98%, 99%, or 100% sequence identity to SEQ ID NO: 5. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOs: 6-12, 15-22, and 24-34.

The nucleic acid sequences encoding the chimeric receptors can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the chimeric receptors disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any chimeric receptor disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic host, such as a mammalian cells (e.g., COS cells, NIH 3T3 cells, or HeLa cells). These cells are available from many sources, including the American Type Culture Collection (Manassas, VA). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

### Recombinant Cells and Cell Cultures

The nucleic acid of the present disclosure can be introduced into a host cell, such as, for example, a human T lymphocyte, to produce a recombinant or engineered cell containing the nucleic acid molecule. Accordingly, some embodiments of the disclosure relate to methods for making a recombinant or engineered cell, including (a) providing a cell capable of protein expression and (b) contacting the provided cell with a recombinant nucleic acid of the disclosure.

Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell (TH), a cytotoxic T cell (TCTL), or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+ T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a subject. In some embodiments, the subject is a human patient.

In some embodiments, the recombinant cell further includes a first and a second nucleic acid molecule as disclosed herein, wherein the first nucleic acid molecule and the second nucleic acid molecule do not have the same sequence. In some embodiments, the recombinant cell further includes a first and a second chimeric receptor as disclosed herein, wherein the first chimeric receptor and the second chimeric receptor do not have the same sequence. In some embodiments, the first chimeric receptor modulates the expression and/or activity of the second chimeric receptor.

In some embodiments, the recombinant cell further includes an expression cassette encoding a protein of interest operably linked to a promoter, wherein expression of the protein of interest is modulated by the chimeric receptor transcriptional regulator. Any suitable promoter can be used in connection with the present disclosure. In some embodiments, the promoter comprises a yeast GAL4 promoter. In some embodiments, the protein of interest is heterologous to the recombinant cell. A heterologous protein is one that is not normally found in the cell, e.g., not normally produced by the cell. In principle, there are no particular limitations with regard to suitable proteins whose expression can be modulated by the chimeric receptor transcriptional regulator. Exemplary types of proteins suitable for use with the compositions and methods disclosed herein include cytokines, cytotoxins, chemokines, immunomodulators, pro-apoptotic factors, anti-apoptotic factors, hormones, differentiation factors, dedifferentiation factors, immune cell receptors, or reporters. In some embodiments, the immune cell receptor is a T-cell receptor (TCR). In some embodiments, the immune cell receptor is a chimeric antigen receptor (CAR). In some embodiments, the expression cassette encoding the protein of interest is incorporated into the same nucleic acid molecule that encodes the chimeric receptor of the disclosure. In some embodiments, the expression cassette encoding the protein of interest is incorporated into a second expression vector that is separate from the nucleic acid molecule encoding the chimeric receptor of the disclosure.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the nucleic acids, and recombinant cells of the disclosure can be incorporated into compositions, including pharmaceutical compositions. Such compositions generally include the nucleic acids, and/or recombinant cells, and a pharmaceutically acceptable excipient, e.g., carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the chimeric receptors and Notch receptors of the disclosure can also be administered by transfection or infection using methods known in the art, including but not limited to the methods described in McCaffrey et al. (Nature 418:6893, 2002), Xia et al. (Nature Biotechnol. 20:1006-10, 2002), or Putnam (Am. J. Health Syst. Pharm. 53:151-60, 1996, erratum at Am. J. Health Syst. Pharm. 53:325, 1996).

### Methods of the Disclosure

Administration of any one of the therapeutic compositions described herein, e.g., nucleic acids, recombinant cells, and pharmaceutical compositions, can be used to treat patients for relevant health conditions or diseases, such as cancers and chronic infections. In some embodiments, the nucleic acids, recombinant cells, and pharmaceutical compositions described herein can be incorporated into therapeutic agents for use in methods of treating an individual who has, who is suspected of having, or who may be at high risk for developing one or more autoimmune disorders or diseases associated with checkpoint inhibition. Exemplary autoimmune disorders and diseases can include, without limitation, celiac disease, type 1 diabetes, Graves' disease, inflammatory bowel disease, multiple sclerosis, psoriasis, rheumatoid arthritis, and systemic lupus erythematosus.

Accordingly, in one aspect, some embodiments of the disclosure relate to methods for inhibiting an activity of a target cell in an individual, the methods include administering to the individual a first therapy including one or more of nucleic acids, recombinant cells, and pharmaceutical compositions as disclosed herein, wherein the first therapy inhibits the target cell. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit an activity of the target cells in the individual. Generally, the target cells of the disclosed methods can be any cell type in an individual and can be, for example an acute myeloma leukemia cell, a myelogenous leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

In another aspect, some embodiments of the disclosure relate to methods for the treatment of a health condition (e.g., disease) in an individual in need thereof, the methods include administering to the individual a first therapy including one or more of the recombinant cells including a chimeric receptor as disclosed herein, and/or pharmaceutical compositions as disclosed herein, wherein the first therapy treats the health condition in the individual. In some embodiments, the methods include administering to the individual a first therapy including an effective number of the recombinant cells as disclosed herein, wherein the recombinant cells treat the health condition.

In another aspect, some embodiments of the disclosure relate to methods for assisting in the treatment of a health condition (e.g., disease) in an individual in need thereof, the methods including administering to the individual a first therapy including one or more of chimeric receptors, HingeNotch receptors, nucleic acids, recombinant cells, and pharmaceutical compositions as disclosed herein, and a second therapy, wherein the first and second therapies together treat the disease in the individual. In some embodiments, the methods include administering to the individual a first therapy including an effective number of the recombinant cells as disclosed herein, wherein the recombinant cells treat the health condition.

### Administration of Recombinant Cells to an Individual

In some embodiments, the methods of the disclosure involve administering an effective amount of the recombinants cells of the disclosure to an individual in need of such treatment. This administering step can be accomplished using any method of implantation delivery in the art. For example, the recombinant cells of the disclosure can be infused directly in the individual's bloodstream or otherwise administered to the individual.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even the lifetime of the individual, i.e., long-term engraftment.

When provided prophylactically, the recombinant cells described herein can be administered to an individual in advance of any symptom of a disease or condition to be treated. Accordingly, in some embodiments the prophylactic administration of a recombinant cell population prevents the occurrence of symptoms of the disease or condition.

When provided therapeutically in some embodiments, recombinant cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source. In some embodiments, the recombinant cells are expanded in culture prior to administration to an individual in need thereof.

In some embodiments, the delivery of a recombinant cell composition (e.g., a composition including a plurality of recombinant cells according to any of the cells described herein) into an individual by a method or route results in at least partial localization of the cell composition at a desired site. A composition including recombinant cells can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a preferred mode of administration.

In some embodiments, the recombinant cells are administered systemically, e.g., via infusion or injection. For example, a population of recombinant cells are administered other than directly into a target site, tissue, or organ, such that it enters, the individual's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. Efficacy can also be measured by failure of an individual to worsen as assessed by decreased hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular beneficial effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments of the disclosed methods, the individual is a mammal. In some embodiments, the mammal is a human. In some embodiments, the individual has or is suspected of having a disease associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection.

Methods for CAR design, delivery and expression in T cells, and the manufacturing of clinical-grade T cell populations are known in the art. See, for example, Lee et al., Clin Cancer Res (2012) 18(10):2780-90. For example, the engineered CARs may be introduced into T cells using retroviruses, which efficiently and stably integrate a nucleic acid sequence encoding the chimeric antigen receptor into the target cell genome. An exemplary method is described in Example 2 below.

Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8al, Cas8a2, Cas8b, Cas8c, Cas9, Cas10, Cas10d, Cas12a (Cpf1), Cas13a (C2c2), Cas13b, Cas13d, CasF, CasG, CasH, Csy1, Csy2, Csy3, Cse1 (or CasA), Cse2 (or CasB), Cse3 (or CasE), CasX, CasY, Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.).

In some embodiments, a recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (e.g., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692.

The T cells, once they have been expanded ex vivo in response to, for example, an autoimmune disease antigen, can be reinfused into the subject in a therapeutically effective amount.

The precise amount of cells to be administered can be determined by a physician with consideration of individual differences in age, weight, extent of disease and condition of the subject.

Administration of T cell therapies may be defined by number of total cells per infusion or number of cells per kilogram of body weight, especially for pediatric subjects (e.g., patients). As T cells replicate and expand after transfer, the administered cell dose may not resemble the final steady-state number of cells. In some embodiments, a pharmaceutical composition including the T cells of the present disclosure may be administered at a dosage of 104 to 1010 total cells. In another embodiment, a pharmaceutical composition including the T cells of the present disclosure may be administered at a dosage of 10³ to 10⁸ cells/kg body weight, including all integer values within those ranges.

Compositions including the T cells of the present disclosure may also be administered multiple times at these dosages. The cells can be administered by using infusion techniques that are known in the art (see, for example, Rosenberg et al., New Engl J Med, (1988) 319:1676). The optimal dosage and treatment regimen for a particular subject can be determined by one skilled in the art by monitoring the subject for signs of disease and adjusting the treatment accordingly.

In some embodiments, administration of any of the compositions embodied herein, for the treatment of, for example, an autoimmune or inflammatory disease, can be combined with other cell-based therapies, for example, stem cells, antigen presenting cells, pancreatic islets etc.

The composition of the present disclosure may be prepared in a manner known in the art and in a manner suitable for parenteral administration to mammals, particularly humans, including a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the present disclosure may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the T cells. In general, the compositions are prepared by uniformly and intimately bringing into association any active ingredients with liquid carriers.

In some embodiments, the composition is suitable for parenteral administration. In another embodiment, the composition is suitable for intravenous administration.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

### Additional Therapies

As discussed above, any one of the compositions as disclosed herein, e.g., the chimeric receptors, recombinant nucleic acids, recombinant cells, cell cultures, and pharmaceutical compositions described herein can be administered to a subject in need thereof as a single therapy (e.g., monotherapy). In addition or alternatively, in some embodiments of the disclosure, the chimeric receptors, recombinant nucleic acids, recombinant cells, cell cultures, and pharmaceutical compositions described herein can be administered to the subject in combination with one or more additional therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

The present disclosure also contemplates the combination of the composition of the disclosure with other drugs and/or in addition to other treatment regimens or modalities such as surgery. When the composition of the present disclosure is used in combination with known therapeutic agents the combination may be administered either in sequence (either continuously or broken up by periods of no treatment) or concurrently or as an admixture. In the case of, for example, autoimmune diseases, treatment includes administering to the subject the compositions embodied herein, e.g. autologous T cells transduced or contacted with a CAR embodied herein and one or more anti-inflammatory agents and/or therapeutic agents. The anti-inflammatory agents include one or more antibodies which specifically bind to pro-inflammatory cytokines, e.g., pro-inflammatory cytokines such as IL-1, TNF, IL-6, GM-CSF, and IFN-γ. In some embodiments, the antibodies are anti-TNFα, anti-IL-6 or combinations thereof. In some embodiments, one or more agents, other than antibodies can be administered which decrease pro-inflammatory cytokines, e.g. non-steroidal anti-inflammatory drugs (NSAIDs). Any combination of antibodies and one or more agents can be administered which decrease pro-inflammatory cytokines.

Treatment in combination is also contemplated to encompass the treatment with either the composition of the disclosure followed by a known treatment, or treatment with a known agent followed by treatment with the composition of the disclosure, for example, as maintenance therapy. For example, in the treatment of autoimmune diseases, excessive and prolonged activation of immune cells, such as T and B lymphocytes, and overexpression of the master pro-inflammatory cytokine tumor necrosis factor alpha (TNF), together with other mediators such as interleukin-6 (TL-6), interleukin-1 (IL-1), and interferon gamma (IFN-γ), play a central role in the pathogenesis of autoimmune inflammatory responses in rheumatoid arthritis (RA), inflammatory bowel disease (IBD), Crohn's disease (CD), and ankylosing spondylitis (AS).

Non-steroidal anti-inflammatory drugs (NSAIDs), glucocorticoids, disease-modifying anti-rheumatic drugs (DMARDs) are traditionally used in the treatment of autoimmune inflammatory diseases. NSAIDs and glucocorticoids are effective in the alleviation of pain and inhibition of inflammation, while DMARDs have the capacity of reducing tissue and organ damage caused by inflammatory responses. More recently, treatment for RA and other autoimmune diseases has been revolutionized with the discovery that TNF is critically important in the development of the diseases. Anti-TNF biologics (such as infliximab, adalimumab, etanercept, golimumab, and certolizumab pepol) have markedly improved the outcome of the management of autoimmune inflammatory diseases.

Non-steroidal anti-inflammatory drugs have the analgesic, antipyretic, and anti-inflammatory effect, frequently used for the treatment of conditions like arthritis and headaches. NSAIDs relieve pain through blocking cyclooxygenase (COX) enzymes. COX promotes the production of prostaglandins, a mediator which causes inflammation and pain. Although NSAIDs have different chemical structures, all of them have the similar therapeutic effect, e.g., inhibition of autoimmune inflammatory responses. In general, NSAIDs can be divided into two broad categories: traditional non-selective NSAIDs and selective cyclooxygenase-2 (COX-2) inhibitors (For a review, see, P. Li et al., Front Pharmacol (2017) 8:460).

In addition to anti-TNF agents, the biologics targeting other proinflammatory cytokines or immune competent molecules have also been extensively studied and actively developed. For example, abatacept, a fully humanized fusion protein of extracellular domain of CTLA-4 and Fc fraction of IgG1, has been approved for the RA patients with inadequate response to anti-TNF therapy. The major immunological mechanism of abatacept is selective inhibition of co-stimulation pathway (CD80 and CD86) and activation of T cells. Tocilizumab, a humanized anti-IL-6 receptor monoclonal antibody was approved for RA patients intolerant to DMARDs and/or anti-TNF biologics. This therapeutic mAb blocks the transmembrane signaling of IL-6 through binding with soluble and membrane forms of TL-6 receptor. Biological drugs targeting TL-1 (anakinra), Th1 immune responses (IL-12/IL-23, ustekinumab), Th17 immune responses (IL-17, secukinumab) and CD20 (rituximab) have also been approved for the treatment of autoimmune diseases (For a review see, P. Li et al., Front Pharmacol (2017) 8:460).

Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Methods for Modulating an Activity of a Cell

In another aspect, provided herein are various methods for modulating an activity of a cell. The methods include the steps of: (a) providing an effective amount of any of the recombinant cells provided herein, and (b) contacting it with a selected ligand, wherein binding of the selected ligand to the extracellular ligand-binding domain induces cleavage of a ligand-inducible proteolytic cleavage site and releases the intracellular domain comprising the intracellular signaling domain and the transcriptional regulator, wherein the released intracellular signaling domain and the transcriptional regulator modulates an activity of the recombinant cell. One skilled in the art upon reading the present disclosure will appreciate that the disclosed methods can be carried out in vivo, ex vivo, or in vitro.

Non-limiting exemplary cellular activities that can be modulated using the methods provide herein include, but are not limited to, gene expression, proliferation, apoptosis, non-apoptotic death, differentiation, dedifferentiation, migration, secretion of a gene product, cellular adhesion, and cytolytic activity.

In some embodiments, the released transcriptional regulator modulates expression of a gene product of the cell. In some embodiments, the released transcriptional regulator modulates expression of a heterologous gene product in the cell. A heterologous gene product is one that is not normally found in the native cell, e.g., not normally produced by the cell. For example, the cell can be genetically modified with a nucleic acid including a nucleotide sequence encoding the heterologous gene product.

In some embodiments, the heterologous gene product is a secreted gene product. In some embodiments, the heterologous gene product is a cell surface gene product. In some cases, the heterologous gene product is an intracellular gene product. In some embodiments, the released transcriptional regulator simultaneously modulates expression of two or more heterologous gene products in the cell.

In some embodiments, the heterologous gene product in the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, an RNA guided nuclease, a site-specific nuclease, a T-cell receptor (TCR), a chimeric antigen receptor (CAR), a toxin, a toxin-derived protein, a transcriptional regulator, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

In some embodiments, the released transcriptional regulator modulates differentiation of the cell, and wherein the cell is an immune cell, a stem cell, a progenitor cell, or a precursor cell.

The chimeric receptors of the disclosure provide a higher degree of expression than a standard SynNotch receptor, when using identical binding domains and ICDs. Depending on the ligand/binding domain pair and their affinity, the chimeric receptors of the disclosure can provide expression enhancement of about 10%, about 15%, about 20%, about 25%, about 30%, about 35%, about 40%, about 45%, or about 50% higher than a corresponding SynNotch receptor.

Additionally, the chimeric receptors of the disclosure can provide transcriptional regulation that responds to the degree of T cell activation, independent of ligand binding. For example, when expressed in a T cell, some receptors of the disclosure provide a stronger ligand-induced signal when the T cell is activated as compared to the ligand-induced signal when the T cell is not activated. This permits additional flexibility in use, for example in cases where it is desired to enhance or suppress a T cell response when activated despite the absence of the chimeric receptor ligand.

### Systems and Kits

Also provided herein are systems and kits including the chimeric receptors, recombinant nucleic acids, recombinant cells, or pharmaceutical compositions provided and described herein as well as written instructions for making and using the same. For example, provided herein, in some embodiments, are systems and/or kits that include one or more of: an chimeric receptor as described herein, a recombinant nucleic acids as described herein, a recombinant cell as described herein, or a pharmaceutical composition as described herein. In some embodiments, the systems and/or kits of the disclosure further include one or more syringes (including pre-filled syringes) and/or catheters (including pre-filled syringes) used to administer one any of the provided chimeric receptors recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a health condition (e.g., disease) in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose. All publications and patent applications mentioned in this disclosure are herein incorporated by reference to the same extent as if each individual publication or patent application was specifically and individually indicated to be incorporated by reference.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1: Design of Human or Humanized Synnotch Reporters

This Example shows the design and construction of the exemplary human or humanized SynNotch receptors provided herein.

Human or humanized Synthetic Notch Receptors were reported in which the transcription factor DNA binding domain (DBD) consists of a zinc finger (ZF) based on a human-derived scaffold, and the transcription factor transactivation domain (TAD) consists of the TAD from the human p65 protein. However, in some embodiments, the relatively weak transcription induced by the ZF-p65 transcription factor is insufficient to drive the levels of transgene expression necessary to observe a therapeutic effect.

Exemplary experiments were perform to develop tandem TADs with increased transactivation activity, in which a short human-derived protein sequence was fused to the original p65 TAD to increase its transcriptional output and drive the level of gene induction necessary to observe the required therapeutic performance in vitro.

Exemplary receptors were built by fusing an anti-CD19 scFv or an anti-ALPPL2 scFv to the corresponding receptor scaffold and intracellular tail. All receptors contain an n-terminal CD8a signal peptide (MALPVTALLLPLALLLHAARP, SEQ ID NO: 73) for membrane targeting and a myc-tag (EQKLISEEDL, SEQ ID NO: 74). Receptors were prepared to contain different intracellular domains (ICDs), including: a transcription factor DNA binding domain (DBD), such as various synthetic zinc finger proteins (based on a human-derived scaffold) or PAX6; a transcription factor transactivation domain (TAD), such as TADs of human p65, GR Tau1, p53, or ATF6; and an additional human transcriptional effector, such as TADs of HSF-1, GATA3, p65, HIF1a, GR Tau1, ATF6, ELF3, or p53. Each of the receptors were cloned into a pHR-SIN vector and introduced, together with a cognate inducible BFP vector, into primary human T cells. For the inducible BFP vectors, a pHR-SIN vector bearing a BFP gene followed by a constitutive PGK promoter driving mCitrine (pRay130) was digested with EcoRI and BamHI immediately upstream of the BFP, enabling insertion of a response element (cognate to the given zinc finger or PAX6 DNA-binding domain) followed by the ybTATA minimal promoter. All constructs were cloned via In-fusion cloning (Clontech #ST0345) or NEB HiFi Assembly (NEB E2621L).Transduced T cells were sorted for Myc/mCitrine double-positive events. Within two weeks of sorting, T cells were co-incubated with K562 target cells in a 1:1 ratio and BFP induction was measured via flow cytometry was measured at various time points between 24 and 72 hours.

FIG. 1A shows a sequence alignment of mouse and human p65 transactivation domains. Exemplary reporters were constructed to contain human HSF1 fused to either the full-length p65 TAD or to the human “mouse-homologous region” (MHR). The components of the resulting exemplary human or humanized HingeNotch receptors containing augmented transactivation domains (e.g., transcriptional effectors) are described in Table 1 below.

TABLE 1
Receptor components description for human or humanized HingeNotch receptors with augmented transactivation domain.

| Plasmid | Receptor | | N- | | | | | | | Transcriptional |
|---|---|---|---|---|---|---|---|---|---|---|
| ID | description | ECD | JMD | TMD | STS | DBD | Linker | TAD | Linker | Effector |
| pDP1179 | HingeNotch- | CD8a signal | trun- | Notch1 | Notch2 | synthetic | Synthetic | mouse | N/A | N/A |
| | ZF3 with mouse | peptide, | cated | | | zinc | short | p65 | | |
| | p65 TAD | myc-tag, | CD8 | | | finger | linker | TAD | | |
| | | anti-CD19 | hinge | | | protein | | | | |
| | | scFv | | | | ZF3 | | | | |
| pDP1180 | HingeNotch- | CD8a signal | trun- | Notch1 | Notch2 | synthetic | Synthetic | human | N/A | N/A |
| | ZF3 with | peptide, | cated | | | zinc | short | p65 | | |
| | mouse- | myc-tag, | CD8 | | | finger | linker | TAD | | |
| | homologous | anti-CD19 | hinge | | | protein | | (mouse- | | |
| | region of human | scFv | | | | ZF3 | | homologous | | |
| | p65 TAD | | | | | | | region) | | |
| pDP1181 | HingeNotch- | CD8a signal | trun- | Notch1 | Notch2 | synthetic | Synthetic | human | Synthetic | human |
| | ZF3 with human | peptide, | cated | | | zinc | short | p65 | Linker | HSF1 |
| | p65 TAD fused | myc-tag, | CD8 | | | finger | linker | TAD | | TAD |
| | to human HSF1 | anti-CD19 | hinge | | | protein | | | | |
| | TAD | scFv | | | | ZF3 | | | | |
| pDP1182 | HingeNotch- | CD8a signal | trun- | Notch1 | Notch2 | synthetic | Synthetic | human | Synthetic | human |
| | ZF3 with | peptide, | cated | | | zinc | short | p65 | Linker | HSF1 |
| | mouse- | myc-tag, | CD8 | | | finger | linker | TAD | | TAD |
| | homologous | anti-CD19 | hinge | | | protein | | (mouse- | | |
| | region of human | scFv | | | | ZF3 | | homologous | | |
| | p65 TAD fused | | | | | | | region) | | |
| | to human HSF1 | | | | | | | | | |
| | TAD | | | | | | | | | |
| pDP1160 | truncated CD8 | CD8a signal | trun- | Notch1 | Notch2 | synthetic | Synthetic | human | N/A | N/A |
| | Hinge receptor | peptide, | cated | | | zinc | short | p65 | | |
| | (HingeNotch) | myc-tag, | CD8 | | | finger | linker | TAD | | |
| | with Notch1 | anti-CD19 | hinge | | | protein | | | | |
| | TMD and | scFv | | | | ZF6 | | | | |
| | Notch2 STS, | | | | | | | | | |
| | with ZF6 | | | | | | | | | |
| | replacing | | | | | | | | | |
| | G4VP64 | | | | | | | | | |
| pDP1174 | HingeNotch- | CD8a signal | trun- | Notch1 | Notch2 | synthetic | Synthetic | mouse | Synthetic | human |
| | ZF6 with mouse | peptide, | cated | | | zinc | short | p65 | Linker | HSF1 |
| | p65 TAD fused | myc-tag, | CD8 | | | finger | linker | TAD | | TAD |
| | to human HSF1 | anti-CD19 | hinge | | | protein | | | | |
| | TAD | scFv | | | | ZF6 | | | | |
| pDP1279 | HingeNotch- | CD8a signal | trun- | Notch1 | Notch2 | synthetic | Synthetic | human | Synthetic | human |
| | ZF6 with human | peptide, | cated | | | zinc | short | p65 | Linker | GATA3 |
| | p65 TAD fused | myc-tag, | CD8 | | | finger | linker | TAD | | TAD1 |
| | to human | anti-CD19 | hinge | | | protein | | | | |
| | GATA3 TAD1 | scFv | | | | ZF6 | | | | |
| pDP1280 | HingeNotch- | CD8a signal | trun- | Notch1 | Notch2 | synthetic | Synthetic | human | Synthetic | human |
| | ZF6 with human | peptide, | cated | | | zinc | short | p65 | Linker | p53 |
| | p65 TAD fused | myc-tag, | CD8 | | | finger | linker | TAD | | TAD1 |
| | to human p53 | anti-CD19 | hinge | | | protein | | | | |
| | TAD1 | scFv | | | | ZF6 | | | | |
| pDP1281 | HingeNotch- | CD8a signal | trun- | Notch1 | Notch2 | synthetic | Synthetic | human | Synthetic | human |
| | ZF6 with human | peptide, | cated | | | zinc | short | p65 | Linker | p53 |
| | p65 TAD fused | myc-tag, | CD8 | | | finger | linker | TAD | | TAD2 |
| | to human p53 | anti-CD19 | hinge | | | protein | | | | |
| | TAD2 | scFv | | | | ZF6 | | | | |
| pDP1285 | HingeNotch- | CD8a signal | trun- | Notch1 | Notch2 | synthetic | Synthetic | human | Synthetic | human |
| | ZF6 with human | peptide, | cated | | | zinc | short | p65 | Linker | HIF1a |
| | p65 TAD fused | myc-tag, | CD8 | | | finger | linker | TAD | | CAD |
| | to human HIF1a | anti-CD19 | hinge | | | protein | | | | |
| | CAD | scFv | | | | ZF6 | | | | |
| pDP1286 | HingeNotch- | CD8a signal | trun- | Notch1 | Notch2 | synthetic | Synthetic | human | Synthetic | human |
| | ZF6 with human | peptide, | cated | | | zinc | short | p65 | Linker | GR Tau1 |
| | p65 TAD fused | myc-tag, | CD8 | | | finger | linker | TAD | | (minimal) |
| | to human GR | anti-CD19 | hinge | | | protein | | | | TAD |
| | Tau1 (Minimal) | scFv | | | | ZF6 | | | | |
| | TAD | | | | | | | | | |
| pDP1290 | HingeNotch- | CD8a signal | trun- | Notch1 | Notch2 | synthetic | Synthetic | human | Synthetic | human |
| | ZF6 with human | peptide, | cated | | | zinc | short | p65 | Linker | ATF6 |
| | p65 TAD fused | myc-tag, | CD8 | | | finger | linker | TAD | | 7aa TAD |
| | to 5 repeats of | anti-CD19 | hinge | | | protein | | | | (5x |
| | human ATF6 | scFv | | | | ZF6 | | | | repeats) |
| | TAD 7aa | | | | | | | | | |
| | fragment | | | | | | | | | |
| pDP1292 | HingeNotch- | CD8a signal | trun- | Notch1 | Notch2 | synthetic | Synthetic | human | Synthetic | human |
| | ZF6 with human | peptide, | cated | | | zinc | short | p65 | Linker | ELF3 |
| | p65 TAD fused | myc-tag, | CD8 | | | finger | linker | TAD | | TAD (2x |
| | to 2 repeats of | anti-CD19 | hinge | | | protein | | | | repeats) |
| | human ELF3 | scFv | | | | ZF6 | | | | |
| | TAD | | | | | | | | | |
| pDP1338 | HingeNotch | CD8a signal | trun- | Notch1 | Notch2 | synthetic | N/A | human | N/A | N/A |
| | with | peptide, | cated | | | zinc | | p65 | | |
| | ZF6/SV40NLS/p65 | myc-tag, | CD8 | | | finger | | TAD | | |
| | SynTF | anti- | hinge | | | protein | | | | |
| | | ALPPL2 | | | | ZF6 | | | | |
| | | scFv | | | | | | | | |
| pDP1339 | HingeNotch | CD8a signal | trun- | Notch1 | Notch2 | synthetic | N/A | human | Synthetic | human |
| | with | peptide, | cated | | | zinc | | p65 | Linker | HSF1 |
| | ZF6/SV40NLS/p65- | myc-tag, | CD8 | | | finger | | TAD | | TAD |
| | HSF1 | anti- | hinge | | | protein | | | | |
| | SynTF | ALPPL2 | | | | ZF6 | | | | |
| | | scFv | | | | | | | | |
| pDP1340 | HingeNotch | CD8a signal | trun- | Notch1 | Notch2 | synthetic | N/A | human | Synthetic | human |
| | with | peptide, | cated | | | zinc | | p65 | Linker | GR Tau1 |
| | ZF6/SV40NLS/p65- | myc-tag, | CD8 | | | finger | | TAD | | (minimal) |
| | GR SynTF | anti- | hinge | | | protein | | | | TAD |
| | | ALPPL2 | | | | ZF6 | | | | |
| | | scFv | | | | | | | | |
| pDP1341 | HingeNotch | CD8a signal | trun- | Notch1 | Notch2 | synthetic | N/A | human | N/A | N/A |
| | with | peptide, | cated | | | zinc | | p65 | | |
| | ZF6/Notch1NLS/p65 | myc-tag, | CD8 | | | finger | | TAD | | |
| | SynTF | anti- | hinge | | | protein | | | | |
| | | ALPPL2 | | | | ZF6 | | | | |
| | | scFv | | | | | | | | |
| pDP1342 | HingeNotch | CD8a signal | trun- | Notch1 | Notch2 | synthetic | N/A | human | Synthetic | human |
| | with | peptide, | cated | | | zinc | | p65 | Linker | HSF1 |
| | ZF6/Notch1NLS/p65- | myc-tag, | CD8 | | | finger | | TAD | | TAD |
| | HSF1 | anti- | hinge | | | protein | | | | |
| | SynTF | ALPPL2 | | | | ZF6 | | | | |
| | | scFv | | | | | | | | |
| pDP1343 | HingeNotch | CD8a signal | trun- | Notch1 | Notch2 | synthetic | N/A | human | Synthetic | human |
| | with | peptide, | cated | | | zinc | | p65 | Linker | GR Tau1 |
| | ZF6/Notch1NLS/p65- | myc-tag, | CD8 | | | finger | | TAD | | (minimal) |
| | GR | anti- | hinge | | | protein | | | | TAD |
| | SynTF | ALPPL2 | | | | ZF6 | | | | |
| | | scFv | | | | | | | | |
| pDP1344 | HingeNotch | CD8a signal | trun- | Notch1 | Notch2 | synthetic | N/A | human | N/A | N/A |
| | with | peptide, | cated | | | zinc | | p65 | | |
| | ZF10/SV40NLS/p65 | myc-tag, | CD8 | | | finger | | TAD | | |
| | SynTF | anti- | hinge | | | protein | | | | |
| | | ALPPL2 | | | | ZF10 | | | | |
| | | scFv | | | | | | | | |
| pDP1345 | HingeNotch | CD8a signal | trun- | Notch1 | Notch2 | synthetic | N/A | human | Synthetic | human |
| | with | peptide, | cated | | | zinc | | p65 | Linker | HSF1 |
| | ZF10/SV40NLS/p65- | myc-tag, | CD8 | | | finger | | TAD | | TAD |
| | HSF1 | anti- | hinge | | | protein | | | | |
| | Syn TF | ALPPL2 | | | | ZF10 | | | | |
| | | scFv | | | | | | | | |
| pDP1346 | HingeNotch | CD8a signal | trun- | Notch1 | Notch2 | synthetic | N/A | human | Synthetic | human |
| | with | peptide, | cated | | | zinc | | p65 | Linker | GR Tau1 |
| | ZF10/SV40NLS/p65- | myc-tag, | CD8 | | | finger | | TAD | | (minimal) |
| | GR SynTF | anti- | hinge | | | protein | | | | TAD |
| | | ALPPL2 | | | | ZF10 | | | | |
| | | scFv | | | | | | | | |
| pDP1347 | HingeNotch | CD8a signal | trun- | Notch1 | Notch2 | synthetic | N/A | human | N/A | N/A |
| | with | peptide, | cated | | | zinc | | p65 | | |
| | ZF10/Notch1NLS/p65 | myc-tag, | CD8 | | | finger | | TAD | | |
| | SynTF | anti- | hinge | | | protein | | | | |
| | | ALPPL2 | | | | ZF10 | | | | |
| | | scFv | | | | | | | | |
| pDP1348 | HingeNotch | CD8a signal | trun- | Notch1 | Notch2 | synthetic | N/A | human | Synthetic | human |
| | with | peptide, | cated | | | zinc | | p65 | Linker | HSF1 |
| | ZF10/Notch1NLS/p65- | myc-tag, | CD8 | | | finger | | TAD | | TAD |
| | HSF1 | anti- | hinge | | | protein | | | | |
| | SynTF | ALPPL2 | | | | ZF10 | | | | |
| | | scFv | | | | | | | | |
| pDP1349 | HingeNotch | CD8a signal | trun- | Notch1 | Notch2 | synthetic | N/A | human | Synthetic | human |
| | with | peptide, | cated | | | zinc | | p65 | Linker | GR Tau1 |
| | ZF10/Notch1NLS/p65- | myc-tag, | CD8 | | | finger | | TAD | | (minimal) |
| | GR | anti- | hinge | | | protein | | | | TAD |
| | SynTF | ALPPL2 | | | | ZF10 | | | | |
| | | scFv | | | | | | | | |
| pRay451A | HingeNotch- | CD8a signal | trun- | Notch1 | Notch2 | Pax6 | | human | Synthetic | human |
| | Pax6 with | peptide, | cated | | | | | p65 | Linker | GR Tau1 |
| | minimized | myc-tag, | CD8 | | | | | TAD, | | (minimal) |
| | human p65TAD | anti-CD19 | hinge | | | | | minimized | | TAD |
| | fused to human | scFv | | | | | | TAD1 | | |
| | GR Tau1 | | | | | | | and | | |
| | (Minimal) TAD | | | | | | | TAD2 | | |
| pRay451B | HingeNotch- | CD8a signal | trun- | Notch1 | Notch2 | Pax6 | | human | N/A | human |
| | Pax6 with | peptide, | cated | | | | | p65 | | GR Tau1 |
| | minimized | myc-tag, | CD8 | | | | | TAD, | | (minimal) |
| | human p65TAD | anti-CD19 | hinge | | | | | minimized | | TAD |
| | fused to human | scFv | | | | | | TAD1 | | |
| | GR Tau1 | | | | | | | and | | |
| | (Minimal) TAD | | | | | | | TAD2 | | |
| | minus GS linker | | | | | | | | | |
| pRay451D | HingeNotch- | CD8a signal | trun- | Notch1 | Notch2 | Pax6 | | human | Synthetic | human |
| | Pax6 with | peptide, | cated | | | | | p65 | Linker | GR Tau1 |
| | human p65TAD | myc-tag, | CD8 | | | | | TAD | | (minimal) |
| | fused to human | anti-CD19 | hinge | | | | | | | TAD |
| | GR Tau1 | scFv | | | | | | | | |
| | (Minimal) TAD | | | | | | | | | |
| pRay451E | HingeNotch- | CD8a signal | trun- | Notch1 | Notch2 | Pax6 | | human | N/A | human |
| | Pax6 with | peptide, | cated | | | | | p65 | | GR Tau1 |
| | human p65TAD | myc-tag, | CD8 | | | | | TAD | | (minimal) |
| | fused to human | anti-CD19 | hinge | | | | | | | TAD |
| | GR Tau1 | scFv | | | | | | | | |
| | (Minimal) TAD | | | | | | | | | |
| | minus GS linker | | | | | | | | | |
| pRay451F | HingeNotch- | CD8a signal | trun- | Notch1 | Notch2 | Pax6 | | human | N/A | human |
| | Pax6 with | peptide, | cated | | | | | p65 | | ATF6 |
| | human p65TAD | myc-tag, | CD8 | | | | | TAD | | 7aa TAD |
| | fused to 5 | anti-CD19 | hinge | | | | | | | (5x |
| | repeats of | scFv | | | | | | | | repeats) |
| | human ATF6 | | | | | | | | | |
| | TAD 7aa | | | | | | | | | |
| | fragment | | | | | | | | | |
| pRay451G | HingeNotch- | CD8a signal | trun- | Notch1 | Notch2 | Pax6 | | human | N/A | human |
| | Pax6 with | peptide, | cated | | | | | p65 | | ATF6 |
| | minimized | myc-tag, | CD8 | | | | | TAD, | | 7aa TAD |
| | human p65TAD | anti-CD19 | hinge | | | | | minimized | | (5x |
| | fused to 5 | scFv | | | | | | TAD1 | | repeats) |
| | repeats of | | | | | | | and | | |
| | human ATF6 | | | | | | | TAD2 | | |
| | TAD 7aa | | | | | | | | | |
| | fragment | | | | | | | | | |
| pRay451H | HingeNotch- | CD8a signal | trun- | Notch1 | Notch2 | Pax6 | | human | N/A | human |
| | Pax6 with | peptide, | cated | | | | | p65 | | ATF6 |
| | p65TAD1 fused | myc-tag, | CD8 | | | | | TAD, | | 7aa TAD |
| | to 5 repeats of | anti-CD19 | hinge | | | | | TAD1 | | (5x |
| | human ATF6 | scFv | | | | | | only | | repeats) |
| | TAD 7aa | | | | | | | | | |
| | fragment | | | | | | | | | |
| pRay451I | HingeNotch- | CD8a signal | trun- | Notch1 | Notch2 | Pax6 | | human | Synthetic | human |
| | Pax6 with | peptide, | cated | | | | | p65 | Linker | p53 |
| | human p65TAD | myc-tag, | CD8 | | | | | TAD | | TAD2 |
| | fused to | anti-CD19 | hinge | | | | | | | |
| | p53TAD2 | scFv | | | | | | | | |
| pRay451J | HingeNotch- | CD8a signal | trun- | Notch1 | Notch2 | Pax6 | | human | N/A | human |
| | Pax6 with | peptide, | cated | | | | | GR | | p65 |
| | human p65TAD | myc-tag, | CD8 | | | | | Tau1 | | TAD |
| | fused with GR1 | anti-CD19 | hinge | | | | | (minimal) | | |
| | (Minimized) | scFv | | | | | | TAD | | |
| | fusion N- | | | | | | | | | |
| | terminal to | | | | | | | | | |
| | Pax6DBD | | | | | | | | | |
| pRay451K | HingeNotch- | CD8a signal | trun- | Notch1 | Notch2 | Pax6 | Single | human | | human |
| | Pax6 with | peptide, | cated | | | | aa | p53 | | p65 |
| | human p65TAD | myc-tag, | CD8 | | | | | TAD2 | | TAD |
| | fused with | anti-CD19 | hinge | | | | | | | |
| | p53TAD2 | scFv | | | | | | | | |
| | fusion N- | | | | | | | | | |
| | terminal to | | | | | | | | | |
| | Pax6DBD | | | | | | | | | |
| pRa451L | HingeNotch- | CD8a signal | trun- | Notch1 | Notch2 | Pax6 | | human | N/A | human |
| | Pax6 with | peptide, | cated | | | | | ATF6 | | p65 |
| | minimized | myc-tag, | CD8 | | | | | 8aa | | TAD, |
| | p65TAD fused | anti-CD19 | hinge | | | | | fragment | | minimized |
| | to 4 repeats of | scFv | | | | | | (4x | | TAD1 |
| | ATF6 TAD 8aa | | | | | | | repeats) | | and |
| | fragment | | | | | | | | | TAD2 |
| pRay451M | HingeNotch- | CD8a signal | trun- | Notch1 | Notch2 | Pax6 | | human | N/A | human |
| | Pax6 with | peptide, | cated | | | | | ATF6 | | p65 |
| | human p65TAD | myc-tag, | CD8 | | | | | 8aa | | TAD |
| | fused to 4 | anti-CD19 | hinge | | | | | fragment | | |
| | repeats of ATF6 | scFv | | | | | | (4x | | |
| | TAD 8aa | | | | | | | repeats) | | |
| | fragment | | | | | | | | | |

In addition, the reference sequences from which each of the components listed in Table 1 is derived from are listed in Table 2 below.

TABLE 2
sequence references for human or humanized HingeNotch receptors with augmented transactivation domain.

| Plasmid | Receptor | | | | | |
|---|---|---|---|---|---|---|
| ID | description | ECD | N-JMD | TMD | STS | DBD |
| pDP1179 | aCD19 | Uniprot P01732: | Uniprot | Uniprot | Uniprot | Reference |
| | HingeNotch | AA1-21, Uniprot | P46531: | P46531: | P46531: | synthetic |
| | ZF3 | P01106: AA410- | AA1427- | AA1736- | AA1758- | zinc |
| | mP65 | 419, FMC63 scFv | 1447, | 1757 | 1762 | finger |
| | | sequence | AA1725- | | | protein |
| | | (https://www.ncbi.nlm.nih.gov/pubmed/9566763/) | 1735 | | | |
| pDP1180 | aCD19 | Uniprot P01732: | Uniprot | Uniprot | Uniprot | Reference |
| | HingeNotch | AA1-21, Uniprot | P46531: | P46531: | P46531: | synthetic |
| | ZF3 hP65 | P01106: AA410- | AA1427- | AA1736- | AA1758- | zinc |
| | (mouse- | 419, FMC63 scFv | 1447, | 1757 | 1762 | finger |
| | homologous | sequence | AA1725- | | | protein |
| | region) | (https://www.ncbi.nlm.nih.gov/pubmed/9566763/) | 1735 | | | |
| pDP1181 | aCD19 | Uniprot P01732: | Uniprot | Uniprot | Uniprot | Reference |
| | HingeNotch | AA1-21, Uniprot | P46531: | P46531: | P46531: | synthetic |
| | ZF3 | P01106: AA410- | AA1427- | AA1736- | AA1758- | zinc |
| | hP65- | 419, FMC63 scFv | 1447, | 1757 | 1762 | finger |
| | HSF1 | sequence | AA1725- | | | protein |
| | | (https://www.ncbi.nlm.nih.gov/pubmed/9566763/) | 1735 | | | |
| pDP1182 | aCD19 | Uniprot P01732: | Uniprot | Uniprot | Uniprot | Reference |
| | HingeNotch | AA1-21, Uniprot | P46531: | P46531: | P46531: | synthetic |
| | ZF3 | P01106: AA410- | AA1427- | AA1736- | AA1758- | zinc |
| | hP65(MHR)- | 419, FMC63 scFv | 1447, | 1757 | 1762 | finger |
| | HSF1 | sequence | AA1725- | | | protein |
| | | (https://www.ncbi.nlm.nih.gov/pubmed/9566763/) | 1735 | | | |
| pDP1160 | aCD19 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | p65 | 419, FMC63 scFv | 1447, | 1757 | 1762 | finger |
| | | sequence | AA1725- | | | protein |
| | | (https://www.ncbi.nlm.nih.gov/pubmed/9566763/) | 1735 | | | |
| pDP1174 | aCD19 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | p65-HSF1 | 419, FMC63 scFv | 1447, | 1757 | 1762 | finger |
| | | sequence | AA1725- | | | protein |
| | | (https://www.ncbi.nlm.nih.gov/pubmed/9566763/) | 1735 | | | |
| pDP1279 | aCD19 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | hP65- | 419, FMC63 scFv | 1447, | 1757 | 1762 | finger |
| | GATA3 | sequence | AA1725- | | | protein |
| | TAD1 | (https://www.ncbi.nlm.nih.gov/pubmed/9566763/) | 1735 | | | |
| pDP1280 | aCD19 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | hP65-p53 | 419, FMC63 scFv | 1447, | 1757 | 1762 | finger |
| | TAD1 | sequence | AA1725- | | | protein |
| | | (https://www.ncbi.nlm.nih.gov/pubmed/9566763/) | 1735 | | | |
| pDP1281 | aCD19 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | hP65-p53 | 419, FMC63 scFv | 1447, | 1757 | 1762 | finger |
| | TAD2 | sequence | AA1725- | | | protein |
| | | (https://www.ncbi.nlm.nih.gov/pubmed/9566763/) | 1735 | | | |
| pDP1285 | aCD19 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | hP65- | 419, FMC63 scFv | 1447, | 1757 | 1762 | finger |
| | HIF 1a | sequence | AA1725- | | | protein |
| | CAD | (https://www.ncbi.nlm.nih.gov/pubmed/9566763/) | 1735 | | | |
| pDP1286 | aCD19 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | hP65-GR | 419, FMC63 scFv | 1447, | 1757 | 1762 | finger |
| | Tau1 | sequence | AA1725- | | | protein |
| | Minimal | (https://www.ncbi.nlm.nih.gov/pubmed/9566763/) | 1735 | | | |
| pDP1290 | aCD19 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | hP65- | 419, FMC63 scFv | 1447, | 1757 | 1762 | finger |
| | 5xATF6(DFDLDLL) | sequence | AA1725- | | | protein |
| | | (https://www.ncbi.nlm.nih.gov/pubmed/9566763/) | 1735 | | | |
| pDP1292 | aCD19 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | hP65- | 419, FMC63 scFv | 1447, | 1757 | 1762 | finger |
| | 2xELF3 | sequence | AA1725- | | | protein |
| | | (https://www.ncbi.nlm.nih.gov/pubmed/9566763/) | 1735 | | | |
| pDP1338 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | p65 | 419, anti-ALPPL2 | 1447, | 1757 | 1762 | finger |
| | (Minimized) | scFv sequence | AA1725- | | | protein |
| | | | 1735 | | | |
| pDP1339 | aALPPL2 | Uniprot P01732: | Uniprot | Uniprot | Uniprot | Reference |
| | HingeNotch | AA1-21, Uniprot | P46531: | P46531: | P46531: | synthetic |
| | cZF6 | P01106: AA410- | AA1427- | AA1736- | AA1758- | zinc |
| | p65 HSF1 | 419, anti-ALPPL2 | 1447, | 1757 | 1762 | finger |
| | (Minimized) | scFv sequence | AA1725- | | | protein |
| | | | 1735 | | | |
| pDP1340 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | p65 GR1 | 419, anti-ALPPL2 | 1447, | 1757 | 1762 | finger |
| | (Minimized) | scFv sequence | AA1725- | | | protein |
| | | | 1735 | | | |
| pDP1341 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | p65 | 419, anti-ALPPL2 | 1447, | 1757 | 1762 | finger |
| | (Minimized) | scFv sequence | AA1725- | | | protein |
| | Notch1NLS | | 1735 | | | |
| pDP1342 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | p65 HSF1 | 419, anti-ALPPL2 | 1447, | 1757 | 1762 | finger |
| | (Minimized) | scFv sequence | AA1725- | | | protein |
| | Notch1NLS | | 1735 | | | |
| pDP1343 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF6 | P01106: AA410- | AA1427- | AA1736- | AA1758- | zinc |
| | p65 GR1 | 419, anti-ALPPL2 | 1447, | 1757 | 1762 | finger |
| | (Minimized) | scFv sequence | AA1725- | | | protein |
| | Notch1NLS | | 1735 | | | |
| pDP1344 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF10 | P01106: AA410- | AA1427- | AA1736- | AA1758- | zinc |
| | p65 | 419, anti-ALPPL2 | 1447, | 1757 | 1762 | finger |
| | (Minimized) | scFv sequence | AA1725- | | | protein |
| | | | 1735 | | | |
| pDP1345 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF10 | P01106: AA410- | AA1427- | AA1736- | AA1758- | zinc |
| | p65 HSF1 | 419, anti-ALPPL2 | 1447, | 1757 | 1762 | finger |
| | (Minimized) | scFv sequence | AA1725- | | | protein |
| | | | 1735 | | | |
| pDP1346 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF10 | P01106: AA410- | AA1427- | AA1736- | AA1758- | zinc |
| | p65 GR1 | 419, anti-ALPPL2 | 1447, | 1757 | 1762 | finger |
| | (Minimized) | scFv sequence | AA1725- | | | protein |
| | | | 1735 | | | |
| pDP1347 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF10 | P01106: AA410- | AA1427- | AA1736- | AA1758- | zinc |
| | p65 | 419, anti-ALPPL2 | 1447, | 1757 | 1762 | finger |
| | (Minimized) | scFv sequence | AA1725- | | | protein |
| | Notch1NLS | | 1735 | | | |
| pDP1348 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF10 | P01106: AA410- | AA1427- | AA1736- | AA1758- | zinc |
| | p65 HSF1 | 419, anti-ALPPL2 | 1447, | 1757 | 1762 | finger |
| | (Minimized) | scFv sequence | AA1725- | | | protein |
| | Notch1NLS | | 1735 | | | |
| pDP1349 | aALPPL2 | Uniprot P01732: | Uniprot | Uniprot | Uniprot | Reference |
| | HingeNotch | AA1-21, Uniprot | P46531: | P46531: | P46531: | synthetic |
| | ZF10 | P01106: AA410- | AA1427- | AA1736- | AA1758- | zinc |
| | p65 GR1 | 419, anti-ALPPL2 | 1447, | 1757 | 1762 | finger |
| | (Minimized) | scFv sequence | AA1725- | | | protein |
| | Notch1NLS | | 1735 | | | |
| pRay451A | aCD19 | Uniprot P01732: | Uniprot | Uniprot | Uniprot | Uniprot |
| | HingeNotch | AA1-21, Uniprot | P01732: | P46531: | Q04721: | P26367: |
| | Pax6DBD | P01106: AA410- | AA138- | AA1736- | AA1701- | AA1- |
| | p65(428)- | 419, FMC63 scFv | 164 | 1757 | 1706 | 139 |
| | GSLinker-GR1 | sequence | | | | |
| | (Minimized) | (https://www.ncbi.nlm.nih.gov/pubmed/9566763/) | | | | |
| pRay451B | aCD19 | Uniprot P01732: | Uniprot | Uniprot | Uniprot | Uniprot |
| | HingeNotch | AA1-21, Uniprot | P01732: | P46531: | Q04721: | P26367: |
| | Pax6DBD | P01106: AA410- | AA138- | AA1736- | AA1701- | AA1- |
| | p65(428)- | 419, FMC63 scFv | 164 | 1757 | 1706 | 139 |
| | GR1 | sequence | | | | |
| | (Minimized) | (https://www.ncbi.nlm.nih.gov/pubmed/9566763/) | | | | |
| pRay451D | aCD19 | Uniprot P01732: | Uniprot | Uniprot | Uniprot | Uniprot |
| | HingeNotch | AA1-21, Uniprot | P01732: | P46531: | Q04721: | P26367: |
| | Pax6DBD | P01106: AA410- | AA138- | AA1736- | AA1701- | AA1- |
| | p65(361)- | 419, FMC63 scFv | 164 | 1757 | 1706 | 139 |
| | GSLinker-GR1 | sequence | | | | |
| | (Minimized) | (https://www.ncbi.nlm.nih.gov/pubmed/9566763/) | | | | |
| pRay451E | aCD19 | Uniprot P01732: | Uniprot | Uniprot | Uniprot | Uniprot |
| | HingeNotch | AA1-21, Uniprot | P01732: | P46531: | Q04721: | P26367: |
| | Pax6DBD | P01106: AA410- | AA138- | AA1736- | AA1701- | AA1- |
| | p65(361)-GR1 | 419, FMC63 scFv | 164 | 1757 | 1706 | 139 |
| | (Minimized) | sequence | | | | |
| | | (https://www.ncbi.nlm.nih.gov/pubmed/9566763/) | | | | |
| pRay451F | aCD19 | Uniprot P01732: | Uniprot | Uniprot | Uniprot | Uniprot |
| | HingeNotch | AA1-21, Uniprot | P01732: | P46531: | Q04721: | P26367: |
| | Pax6DBD | P01106: AA410- | AA138- | AA1736- | AA1701- | AA1- |
| | p65(361)- | 419, FMC63 scFv | 164 | 1757 | 1706 | 139 |
| | 5xATF6(DFDLDLL) | sequence | | | | |
| | | (https://www.ncbi.nlm.nih.gov/pubmed/9566763/) | | | | |
| pRay451G | aCD19 | Uniprot P01732: | Uniprot | Uniprot | Uniprot | Uniprot |
| | HingeNotch | AA1-21, Uniprot | P01732: | P46531: | Q04721: | P26367: |
| | Pax6DBD | P01106: AA410- | AA138- | AA1736- | AA1701- | AA1- |
| | p65(428)- | 419, FMC63 scFv | 164 | 1757 | 1706 | 139 |
| | 5xATF6(DFDLDLL) | sequence | | | | |
| | | (https://www.ncbi.nlm.nih.gov/pubmed/9566763/) | | | | |
| pRay451H | aCD19 | Uniprot P01732: | Uniprot | Uniprot | Uniprot | Uniprot |
| | HingeNotch | AA1-21, Uniprot | P01732: | P46531: | Q04721: | P26367: |
| | Pax6DBD | P01106: AA410- | AA138- | AA1736- | AA1701- | AA1- |
| | p65(521)- | 419, FMC63 scFv | 164 | 1757 | 1706 | 139 |
| | 5xATF6(DFDLDLL) | sequence | | | | |
| | | (https://www.ncbi.nlm.nih.gov/pubmed/9566763/) | | | | |
| pRay451I | aCD19 | Uniprot P01732: | Uniprot | Uniprot | Uniprot | Uniprot |
| | HingeNotch | AA1-21, Uniprot | P01732: | P46531: | Q04721: | P26367: |
| | Pax6DBD | P01106: AA410- | AA138- | AA1736- | AA1701- | AA1- |
| | p65(361)- | 419, FMC63 scFv | 164 | 1757 | 1706 | 139 |
| | GSLinker | sequence | | | | |
| | p53TAD2 | (https://www.ncbi.nlm.nih.gov/pubmed/9566763/) | | | | |
| pRay451J | aCD19 | Uniprot P01732: | Uniprot | Uniprot | Uniprot | Uniprot |
| | HingeNotch | AA1-21, Uniprot | P01732: | P46531: | Q04721: | P26367: |
| | GR1 | P01106: AA410- | AA138- | AA1736- | AA1701- | AA1- |
| | (Minimized)- | 419, FMC63 scFv | 164 | 1757 | 1706 | 139 |
| | Pax6DBD- | sequence | | | | |
| | p65(428) | (https://www.ncbi.nlm.nih.gov/pubmed/9566763/) | | | | |
| pRay451K | aCD19 | Uniprot P01732: | Uniprot | Uniprot | Uniprot | Uniprot |
| | HingeNotch | AA1-21, Uniprot | P01732: | P46531: | Q04721: | P26367: |
| | p53TAD2 | P01106: AA410- | AA138- | AA1736- | AA1701- | AA1- |
| | Pax6DBD- | 419, FMC63 scFv | 164 | 1757 | 1706 | 139 |
| | p65(428) | sequence | | | | |
| | | (https://www.ncbi.nlm.nih.gov/pubmed/9566763/) | | | | |
| pRay451L | aCD19 | Uniprot P01732: | Uniprot | Uniprot | Uniprot | Uniprot |
| | HingeNotch | AA1-21, Uniprot | P01732: | P46531: | Q04721: | P26367: |
| | Pax6DBD | P01106: AA410- | AA138- | AA1736- | AA1701- | AA1- |
| | ATF6(DFDLDLLP) | 419, FMC63 scFv | 164 | 1757 | 1706 | 139 |
| | p65(428) | sequence | | | | |
| | | (https://www.ncbi.nlm.nih.gov/pubmed/9566763/) | | | | |
| pRay451M | aCD19 | Uniprot P01732: | Uniprot | Uniprot | Uniprot | Uniprot |
| | HingeNotch | AA1-21, Uniprot | P01732: | P46531: | Q04721: | P26367: |
| | Pax6DBD | P01106: AA410- | AA138- | AA1736- | AA1701- | AA1- |
| | ATF6(DFDLDLLP) | 419, FMC63 scFv | 164 | 1757 | 1706 | 139 |
| | p65(361) | sequence | | | | |
| | | (https://www.ncbi.nlm.nih.gov/pubmed/9566763/) | | | | |

| Plasmid | | | | Transcriptional |
|---|---|---|---|---|
| ID | Linker | TAD | Linker | effector |
| pDP1179 | Derived | UNIPROT | N/A | N/A |
| | from | Q04207: | | |
| | restriction | AA369- | | |
| | sites | 549 | | |
| pDP1180 | Derived | UNIPROT | N/A | N/A |
| | from | Q04206: | | |
| | restriction | AA369- | | |
| | sites | 551 | | |
| pDP1181 | Derived | UNIPROT | Synthetic | UNIPROT |
| | from | Q04206: | Linker | Q00613: |
| | restriction | AA361- | | AA406- |
| | sites | 551 | | 529 |
| pDP1182 | Derived | UNIPROT | Synthetic | UNIPROT |
| | from | Q04206: | Linker | Q00613: |
| | restriction | AA369- | | AA406- |
| | sites | 551 | | 529 |
| pDP1160 | Derived | UNIPROT | N/A | N/A |
| | from | Q04206: | | |
| | restriction | AA361- | | |
| | sites | 551 | | |
| pDP1174 | Derived | UNIPROT | Synthetic | UNIPROT |
| | from | Q04207: | Linker | Q00613: |
| | restriction | AA369- | | AA406- |
| | sites | 549 | | 529 |
| pDP1279 | Derived | UNIPROT | Synthetic | UNIPROT |
| | from | Q04206: | Linker | P23771: |
| | restriction | AA361- | | AA30-74 |
| | sites | 551 | | |
| pDP1280 | Derived | UNIPROT | Synthetic | UNIPROT |
| | from | Q04206: | Linker | P04637: |
| | restriction | AA361- | | AA2-40 |
| | sites | 551 | | |
| pDP1281 | Derived | UNIPROT | Synthetic | UNIPROT |
| | from | Q04206: | Linker | P04637: |
| | restriction | AA361- | | AA41-85 |
| | sites | 551 | | |
| pDP1285 | Derived | UNIPROT | Synthetic | UNIPROT |
| | from | Q04206: | Linker | Q16665: |
| | restriction | AA361- | | AA786- |
| | sites | 551 | | 826 |
| pDP1286 | Derived | UNIPROT | Synthetic | UNIPROT |
| | from | Q04206: | Linker | P04150: |
| | restriction | AA361- | | AA187- |
| | sites | 551 | | 244 |
| pDP1290 | Derived | UNIPROT | Synthetic | Uniprot |
| | from | Q04206: | Linker | P18850(VAR_{—} |
| | restriction | AA361- | | 022455): |
| | sites | 551 | | AA61- |
| | | | | 68 |
| pDP1292 | Derived | UNIPROT | Synthetic | UNIPROT |
| | from | Q04206: | Linker | P78545: |
| | restriction | AA361- | | AA129- |
| | sites | 551 | | 159 |
| pDP1338 | Derived | UNIPROT | N/A | N/A |
| | from | Q04206: | | |
| | restriction | AA361- | | |
| | sites | 551 | | |
| pDP1339 | Derived | UNIPROT | Synthetic | UNIPROT |
| | from | Q04206: | Linker | Q00613: |
| | restriction | AA361- | | AA406- |
| | sites | 551 | | 529 |
| pDP1340 | Derived | UNIPROT | Synthetic | UNIPROT |
| | from | Q04206: | Linker | P04150: |
| | restriction | AA361- | | AA187- |
| | sites | 551 | | 244 |
| pDP1341 | Derived | UNIPROT | N/A | N/A |
| | from | Q04206: | | |
| | restriction | AA361- | | |
| | sites | 551 | | |
| pDP1342 | Derived | UNIPROT | Synthetic | UNIPROT |
| | from | Q04206: | Linker | Q00613: |
| | restriction | AA361- | | AA406- |
| | sites | 551 | | 529 |
| pDP1343 | Derived | UNIPROT | Synthetic | UNIPROT |
| | from | Q04206: | Linker | P04150: |
| | restriction | AA361- | | AA187- |
| | sites | 551 | | 244 |
| pDP1344 | Derived | UNIPROT | N/A | N/A |
| | from | Q04206: | | |
| | restriction | AA361- | | |
| | sites | 551 | | |
| pDP1345 | Derived | UNIPROT | Synthetic | UNIPROT |
| | from | Q04206: | Linker | Q00613: |
| | restriction | AA361- | | AA406- |
| | sites | 551 | | 529 |
| pDP1346 | Derived | UNIPROT | Synthetic | UNIPROT |
| | from | Q04206: | Linker | P04150: |
| | restriction | AA361- | | AA187- |
| | sites | 551 | | 244 |
| pDP1347 | Derived | UNIPROT | N/A | N/A |
| | from | Q04206: | | |
| | restriction | AA361- | | |
| | sites | 551 | | |
| pDP1348 | Derived | UNIPROT | Synthetic | UNIPROT |
| | from | Q04206: | Linker | Q00613: |
| | restriction | AA361- | | AA406- |
| | sites | 551 | | 529 |
| pDP1349 | Derived | UNIPROT | Synthetic | UNIPROT |
| | from | Q04206: | Linker | P04150: |
| | restriction | AA361- | | AA187- |
| | sites | 551 | | 244 |
| pRay451A | N/A | Uniprot | Synthetic | UNIPROT |
| | | Q04206: | | P04150: |
| | | AA428- | | AA187- |
| | | 551 | | 244 |
| pRay451B | N/A | Uniprot | N/A | UNIPROT |
| | | Q04206: | | P04150: |
| | | AA428- | | AA187- |
| | | 551 | | 244 |
| pRay451D | N/A | UNIPROT | Synthetic | UNIPROT |
| | | Q04206: | | P04150: |
| | | AA361- | | AA187- |
| | | 551 | | 244 |
| pRay451E | N/A | Uniprot | N/A | UNIPROT |
| | | Q04206: | | P04150: |
| | | AA361- | | AA187- |
| | | 551 | | 244 |
| pRay451F | N/A | Uniprot | N/A | Uniprot |
| | | Q04206: | | P18850(VAR_{—} |
| | | AA361- | | 022455): |
| | | 551 | | AA61- |
| | | | | 68 |
| pRay451G | N/A | Uniprot | N/A | Uniprot |
| | | Q04206: | | P18850(VAR_{—} |
| | | AA428- | | 022455): |
| | | 551 | | AA61- |
| | | | | 68 |
| pRay451H | N/A | Uniprot | N/A | Uniprot |
| | | Q04206: | | P18850(VAR_{—} |
| | | AA521- | | 022455): |
| | | 551 | | AA61- |
| | | | | 68 |
| pRay451I | N/A | Uniprot | Synthetic | UNIPROT |
| | | Q04206: | | P04637: |
| | | AA361- | | AA41-85 |
| | | 551 | | |
| pRay451J | N/A | UNIPROT | N/A | Uniprot |
| | | P04150: | | Q04206: |
| | | AA187- | | AA428-551 |
| | | 244 | | |
| pRay451K | N/A | UNIPROT | N/A | Uniprot |
| | | P04637: | | Q04206: |
| | | AA41- | | AA428-551 |
| | | 85 | | |
| pRay451L | N/A | Uniprot | N/A | Uniprot |
| | | P18850(VAR_{—} | | Q04206: |
| | | 022455): | | AA428-551 |
| | | AA61-69 | | |
| pRay451M | N/A | Uniprot | N/A | Uniprot |
| | | P18850(VAR_{—} | | Q04206: |
| | | 022455): | | AA361- |
| | | AA61-69 | | 551 |

The sequences of the components of the resulting exemplary human or humanized HingeNotch receptors containing augmented transactivation domains (e.g., transcriptional effectors) are described in Sequence Table after Examples.

### Example 2: Methods

This Example describes the additional methods used in the present disclosure.

### Primary Human T Cell Isolation and Culture

Primary CD4+ and CD8+ T cells were isolated from anonymous donor blood after apheresis by negative selection (STEMCELL Technologies #15062 & 15063). Blood was obtained from Blood Centers of the Pacific (San Francisco, CA) as approved by the University Institutional Review Board. T cells were cryopreserved in RPMI-1640 (UCSF cell culture core) with 20% human AB serum (Valley Biomedical Inc., #HP1022) and 10% DMSO. After thawing, T cells were cultured in human T cell medium consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

### Lentiviral Transduction of Human T Cells

Pantropic VSV-G pseudotyped lentivirus was produced via transfection of Lenti-X 293T cells (Clontech #11131D) with a pHR'SIN:CSW transgene expression vector and the viral packaging plasmids pCMVdR8.91 and pMD2.G using Mirus TransIT-Lenti (Mirus #MTR 6606). Primary T cells were thawed the same day, and after 24 hours in culture, were stimulated with Human T-Activator CD3/CD28 Dynabeads (Life Technologies #11131D) at a 1:3 cell:bead ratio. At 48 hours, viral supernatant was harvested and the primary T cells were exposed to the virus for 24 hours. At day 5 post T cell stimulation, the Dynabeads were removed, T cells were sorted, and the T cells expanded until day 7-14 when they were rested for in vitro assays. T cells were sorted for assays with a Beckton Dickinson (BD) FACs ARIA II.

### Cancer Cell Lines

The cancer cell lines used were K562 myelogenous leukemia cells (ATCC #CCL-243). K562 cells were lentivirally transduced to stably express human CD19. CD19 levels were determined by staining the cells with α-CD19 APC (Biolegend #302212) or BV421 (Biolegend #302234). All cell lines were sorted for expression of the transgenes.

### Circuit Induction Assay

To assess circuit induction, primary human T cells were co-transduced with the human or humanized HingeNotch receptors and a response element containing a UAS-ybTATA promoted BFP gene, transduced cells were then co-cultured with K562 cells, either with or without CD19, at a 1:1 ratio for 48 hours and then analyzed on a flow cytometer to assess expression of BFP. To assess HingeNotch/CAR-mediated killing, T cells and K562 cells were co-cultured for 72 hours and then centrifuged, resuspended in PBS with diluted DRAQ7 for 30 minutes, centrifuged again, and resuspended in PBS without DRAQ7 to assess viability, and then analyzed on a flow cytometer to assess T cell and K562 count and viability.

### Example 3: Human or Humanized Hingenotch Expression and Circuit Induction

This Example demonstrates that the exemplary human or humanized HingeNotch receptors provided herein, with an additional transcriptional effector, are able to activate target gene expression in human T cells.

Briefly, primary human CD4+ T cells were transduced with lentiviral constructs expressing some exemplary HingeNotch receptors, such as CD19-targeting HingeNotch receptors with transcription factors consisting of the ZF3 DNA binding domain and transactivation domains consisting of the human p65 TAD fused to a panel of several different transactivation domain segments, with or without a C-terminal transcriptional effector from any one of: a mouse p65 TAD, a human p65 “mouse-homologous region” (MHR), a human p65 MHR fused to the HSF1 TAD, and a full-length human p65 fused to the HSF1 TAD (FIG. 1). Constructs were co-transduced with a BFP-reporter driven by a ZF3-responsive promoter. To assess circuit induction, T cells expressing different anti-CD19 receptors and the BFP reporter were co-cultured with K562 cells or CD19+K562 cells for 48 hours. Transcriptional activation of the inducible BFP reporter gene was subsequently measured using a Fortessa X-50 (BD).

As demonstrated, all tested human or humanized HingeNotch receptors induced BFP expression specifically upon co-incubation of T cells with CD19-expressing K562 target cells, while those receptors having HSF1 as an additional transcriptional effector to p65 induced higher BFP expression levels than other receptors with only the p65 transcriptional effector (FIG. 1C).

In another similar exemplary experiment, more CD19-targeting HingeNotch receptors bearing the ZF6 DNA binding domain were tested for their capacity to induce BFP expression from a ZF6-responsive promoter in primary human T cells. These receptors contained mouse or human p65 TAD, with or without additional transcriptional effectors, such as 2xELF3, ATF6, GR Tau1 (minimal), HIF1a CAD, p53 TAD2, p53 TAD1, GATA3 TAD1, and human HSF1 (FIG. 2A). As shown in FIG. 2B, all additional transcriptional effectors improved the capacity of HingeNotch receptors to activate BFP expression in response to stimulus by CD19-expressing K562 cells. Notably, some additional transcriptional effectors (e.g., ATF6) resulted in high levels of non-specific activation in response to CD19-deficient (unmodified) K562 cells, while most others (such as GR Tau1 Min) displayed augmented activation relative to p65 alone without introducing significant non-specific “leaky” activation.

### Example 4: Human or Humanized Hingenotch Activates Cells for Target Killing

This Example shows the cell killing activity of the exemplary HingeNotch receptors.

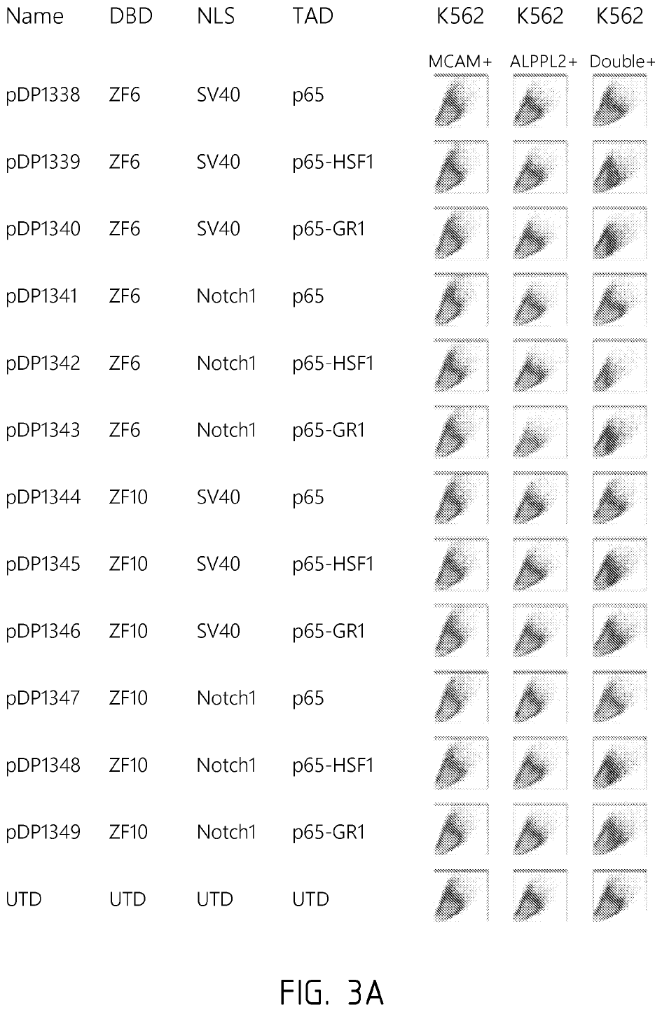

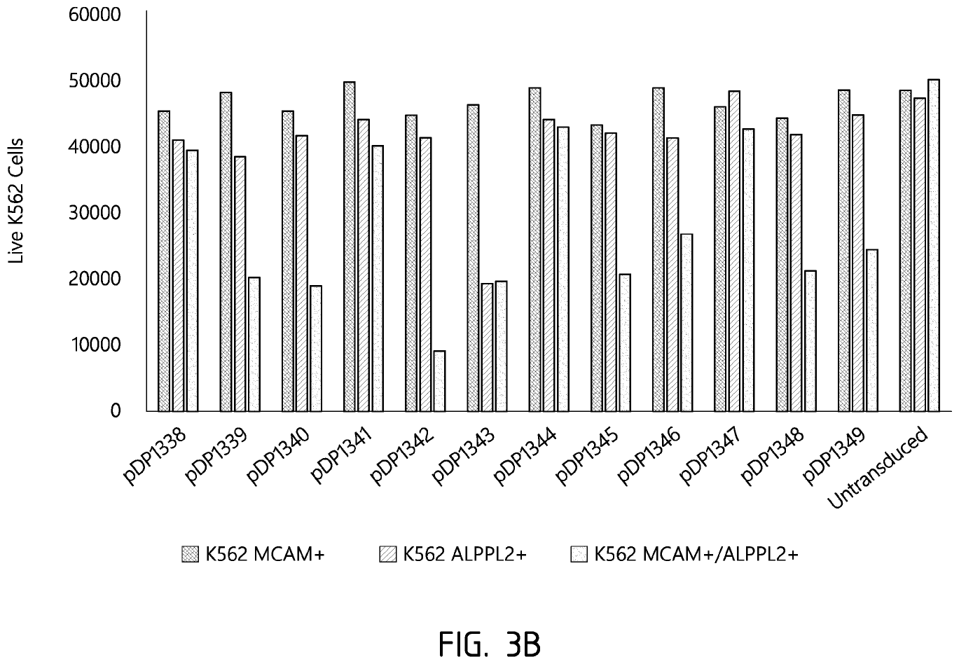

Primary human CD8+ T cells were transduced with a zinc finger-inducible anti-MCAM CAR construct and each of a panel of anti-ALPPL2 receptors with various transcriptional effectors. K562 cells expressing MCAM, ALPPL2, or both MCAM and ALPPL2 ligands were co-incubated with transduced T cells at a 1:1 ratio for 72 hours. Cells were analyzed by FACS for their size and granularity profile (FSC/SSC) (FIG. 3A) and for DRAQ7 fluorescence to detect viability. As shown in FIG. 3B, compared to receptors having a p65 TAD without additional transcriptional effectors (e.g., pDP1338, pDP1341, pDP1344, and pDP1347), adding various additional transcriptional effectors to the receptor constructs significantly improved the target gene expression, leading to T cell activation to kill target K562 cells expressing both different ligands.

More exemplary human or humanized HingeNotch receptors described in Example 1 were tested for their activation and signaling in T cells. A summary of the results is provided in Table 3 below.

TABLE 3
Test Result Description.

| Plasmid ID | Receptor description | T Cell Activation Result |
|---|---|---|
| pDP1179 | HingeNotch-ZF3 with mouse p65 TAD | Similar activation strength as full length human |
| | | p65 (pDP1160) |
| pDP1180 | HingeNotch-ZF3 with mouse-homologous region of | Similar activation strength as full length human |
| | human p65 TAD | p65 (pDP1160) |
| pDP1181 | HingeNotch-ZF3 with human p65 TAD fused to human | Stronger activation strength than full length |
| | HSF1 TAD | human p65 (pDP1160) |
| pDP1182 | HingeNotch-ZF3 with mouse-homologous region of | Stronger activation strength than full length |
| | human p65 TAD fused to human HSF1 TAD | human p65 (pDP1160) |
| pDP1160 | truncated CD8 Hinge receptor (HingeNotch) with Notch1 | Stronger activation than ZF3, minimal residual |
| | TMD and Notch2 STS, with ZF6 replacing G4VP64 | leakage |
| pDP1174 | HingeNotch-ZF6 with mouse p65 TAD fused to human | Stronger activation strength than full length |
| | HSF1 TAD | human p65 (pDP1160) |
| pDP1279 | HingeNotch-ZF6 with human p65 TAD fused to human | Similar activation strength as full length human |
| | GATA3 TAD1 | p65 (pDP1160) |
| pDP1280 | HingeNotch-ZF6 with human p65 TAD fused to human | Similar activation strength as full length human |
| | p53 TAD1 | p65 (pDP1160) |
| pDP1281 | HingeNotch-ZF6 with human p65 TAD fused to human | Stronger activation strength than full length |
| | p53 TAD2 | human p65 (pDP1160) |
| pDP1285 | HingeNotch-ZF6 with human p65 TAD fused to human | Stronger activation strength than full length |
| | HIF1a CAD | human p65 (pDP1160) |
| pDP1286 | HingeNotch-ZF6 with human p65 TAD fused to human | Stronger activation strength than full length |
| | GR Taul (Minimal) TAD | human p65 (pDP1160) |
| pDP1290 | HingeNotch-ZF6 with human p65 TAD fused to 5 repeats | Stronger activation strength than full length |
| | of human ATF6 TAD 7aa fragment | human p65 (pDP1160) |
| pDP1292 | HingeNotch-ZF6 with human p65 TAD fused to 2 repeats | Stronger activation strength than full length |
| | of human ELF3 TAD | human p65 (pDP1160) |
| pDP1338 | HingeNotch with ZF6/SV40NLS/p65 SynTF | Poor target killing |
| pDP1339 | HingeNotch with ZF6/SV40NLS/p65-HSF1 SynTF | Robust target killing |
| pDP1340 | HingeNotch with ZF6/SV40NLS/p65-GR SynTF | Robust target killing |
| pDP1341 | HingeNotch with ZF6/Notch1NLS/p65 SynTF | Poor target killing |
| pDP1342 | HingeNotch with ZF6/Notch1NLS/p65-HSF1 SynTF | Robust target killing |
| pDP1343 | HingeNotch with ZF6/Notch1NLS/p65-GR Syn TF | Robust target killing |
| pDP1344 | HingeNotch with ZF10/SV40NLS/p65 SynTF | Poor target killing |
| pDP1345 | HingeNotch with ZF10/SV40NLS/p65-HSF1 SynTF | Robust target killing |
| pDP1346 | HingeNotch with ZF10/SV40NLS/p65-GR Syn TF | Robust target killing |
| pDP1347 | HingeNotch with ZF10/Notch1NLS/p65 SynTF | Poor target killing |
| pDP1348 | HingeNotch with ZF10/Notch1NLS/p65-HSF1 SynTF | Robust target killing |
| pDP1349 | HingeNotch with ZF10/Notch1NLS/p65-GR SynTF | Robust target killing |
| pRay451A | HingeNotch-Pax6 with minimized human p65TAD fused | Improved activation/target killing over |
| | to human GR Tau1 (Minimal) TAD | p65(428) alone |
| pRay451B | HingeNotch-Pax6 with minimized human p65TAD fused | Improved activation/target killing over |
| | to human GR Tau1 (Minimal) TAD minus GS linker | p65(428) alone |
| pRay451D | HingeNotch-Pax6 with human p65TAD fused to human | Improved activation/target killing over |
| | GR Tau1 (Minimal) TAD | p65(361) alone |
| pRay451E | HingeNotch-Pax6 with human p65TAD fused to human | Improved activation/target killing over |
| | GR Tau1 (Minimal) TAD minus GS linker | p65(361) alone |
| pRay451F | HingeNotch-Pax6 with human p65TAD fused to 5 | Improved activation/target killing over |
| | repeats of human ATF6 TAD 7aa fragment | p65(361) alone |
| pRay451G | HingeNotch-Pax6 with minimized human p65TAD fused | Decreased activation/target killing over |
| | to 5 repeats of human ATF6 TAD 7aa fragment | p65(428) alone |
| pRay451H | HingeNotch-Pax6 with p65TAD1 fused to 5 repeats of | Poor activation/killing |
| | human ATF6 TAD 7aa fragment | |
| pRay451I | HingeNotch-Pax6 with human p65TAD fused to | Improved activation/target killing over |
| | p53TAD2 | p65(361) alone |
| pRay451J | HingeNotch-Pax6 with human p65TAD fused with GR1 | Poor activation/killing |
| | (Minimized) fusion N-terminal to Pax6DBD | |
| pRay451K | HingeNotch-Pax6 with human p65TAD fused with | Poor activation/killing |
| | p53TAD2 fusion N-terminal to Pax6DBD | |
| pRay451L | HingeNotch-Pax6 with minimized p65TAD fused to 4 | Improved activation/target killing over |
| | repeats of ATF6 TAD 8aa fragment | p65(428) alone |
| pRay451M | HingeNotch-Pax6 with human p65TAD fused to 4 | Improved activation/target killing over |
| | repeats of ATF6 TAD 8aa fragment | p65(361) alone |

### Example 5: Methods for Examples 5-9

### Receptor and Response Element Construct Design

Receptors were built by fusing the CD19 scFv {Porter:2011gr} or ALPPL2 scFv (M25^{FYIA}) to the corresponding SynTF obtained from the Khalil laboratory. All receptors contain an n-terminal CD8a signal peptide (MALPVTALLLPLALLLHAARP: SEQ ID NO:73) for membrane targeting and a myc-tag (EQKLISEEDL: SEQ ID NO:74) for facile determination of surface expression with α-myc A647 (Cell-Signaling #2233). The receptors were cloned into a modified pHR′ SIN:CSW vector containing a PGK promoter.

The pHR′ SIN:CSW vector was also modified to make the response element plasmids. Eight copies of the ZF3 (aGACGTCGAAGTAGCCGTAg), ZF6 (gGACGACGCGGTCTAAGAAg), or ZF10 (cGGCGTAGCCGATGTCGCGc) DNA binding domain target sequence were cloned 5′ to a minimal pybTATA promoter driving a tagBFP gene or anti-MCAM (BBz) CAR. Also included in the response element plasmids is a PGK promoter that constitutively drives mCitrine expression to easily identify transduced T cells. For all inducible BFP vectors, BFP was cloned via a BamHI site in the multiple cloning site 3′ to the ybTATA promoter. For response element vectors used with Pax6-based receptors, four copies of a consensus Pax6 binding site, ATTTTCACGCATGAGTGCACAG, were cloned 5′ to a minimal pybTATA promoter driving either a tagBFP gene or a Flag-tagged anti-BCMA (BBz) CAR. All constructs were cloned via Gibson HiFi assembly (NEB #E2621L) or InFusion Assembly (Takara 638920).

TABLE 4
Receptor components description for human or humanized HingeNotch
receptors with augmented transactivation domain.

| Plasmid | Receptor | | | | | | | |
|---|---|---|---|---|---|---|---|---|
| ID | description | ECD | N-JMD | TMD | STS | RAM | NLS | DBD |
| pDP1441 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65 | anti-CD19 | | | | | | ZF6 |
| | SynTF | scFv | | | | | | (SV40NLS) |
| pDP1487 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65- | anti-CD19 | | | | | | ZF6 |
| | GR SynTF | scFv | | | | | | (SV40NLS) |
| pDP1558 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65- | anti-CD19 | | | | | | ZF6 |
| | MIER3 | scFv | | | | | | (SV40NLS) |
| | SynTF | | | | | | | synthetic |
| pDP1560 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65- | anti-CD19 | | | | | | ZF6 |
| | MLXIPL | scFv | | | | | | (SV40NLS) |
| | SynTF | | | | | | | |
| pDP1562 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65- | anti-CD19 | | | | | | ZF6 |
| | NFE2L1 | scFv | | | | | | (SV40NLS) |
| | SynTF | | | | | | | |
| pDP1566 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65- | anti-CD19 | | | | | | ZF6 |
| | PTF1A | scFv | | | | | | (SV40NLS) |
| | SynTF | | | | | | | |
| pDP1673 | HingeNotch | truncated | truncated | Notch1 | Notch1 | N/A | SV40NLS | synthetic |
| | (Notch1 STS), | CD8 hinge | CD8 hinge | | | | | zinc finger |
| | optimized | | | | | | | protein |
| | ZF6/SV40NLS/p65- | | | | | | | ZF6 |
| | MLXIPL | | | | | | | (SV40NLS) |
| | SynTF | | | | | | | |
| pDP1674 | HingeNotch | truncated | truncated | Notch1 | Notch1 | human | SV40NLS | synthetic |
| | (Notch1 STS), 8 | CD8 hinge | CD8 hinge | | | Notch1 | | zinc finger |
| | RAM residues, | | | | | RAM | | protein |
| | optimized | | | | | (8/11) | | ZF6 |
| | ZF6/SV40NLS/p65- | | | | | | | (SV40NLS) |
| | MLXIPL SynTF | | | | | | | |
| pDP1675 | HingeNotch | truncated | truncated | Notch1 | Notch2 | human | SV40NLS | synthetic |
| | (Notch2 STS), 8 | CD8 hinge | CD8 hinge | | | Notch1 | | zinc finger |
| | RAM residues, | | | | | RAM | | protein |
| | optimized | | | | | (8/11) | | ZF6 (SV40 |
| | ZF6/SV40NLS/p65- | | | | | | | NLS) |
| | MLXIPL SynTF | | | | | | | |
| pDP1757 | HingeNotch | CD8a signal | truncated | Notch1 | N/A | N/A | human | synthetic |
| | with 5 residues | peptide, | CD8 hinge | | | | Notch1NLS | zinc finger |
| | truncated | myc-tag, | | | | | | protein |
| | upstream of | anti-CD19 | | | | | | ZF6 |
| | RAM start, | scFv | | | | | | (SV40NLS) |
| | optimized | | | | | | | |
| | ZF6/Notch1NLS/p65- | | | | | | | |
| | MLXIPL SynTF | | | | | | | |
| pDP1758 | HingeNotch | CD8a signal | truncated | Notch1 | Notch1 | N/A | human | synthetic |
| | with 4 residues | peptide, | CD8 hinge | | (Partial) | | Notch1NLS | zinc finger |
| | truncated | myc-tag, | | | | | | protein |
| | upstream of | anti-CD19 | | | | | | ZF6 |
| | RAM start, | scFv | | | | | | (SV40NLS) |
| | optimized | | | | | | | |
| | ZF6/Notch1 | | | | | | | |
| | NLS/p65- | | | | | | | |
| | MLXIPL SynTF | | | | | | | |
| pDP1759 | HingeNotch | CD8a signal | truncated | Notch1 | Notch1 | N/A | human | synthetic |
| | with 3 residues | peptide, | CD8 hinge | | (Partial) | | Notch1NLS | zinc finger |
| | truncated | myc-tag, | | | | | | protein |
| | upstream of | anti-CD19 | | | | | | ZF6 |
| | RAM start, | scFv | | | | | | (SV40NLS) |
| | optimized | | | | | | | |
| | ZF6/Notch1NLS/p65- | | | | | | | |
| | MLXIPL SynTF | | | | | | | |
| pDP1760 | HingeNotch | CD8a signal | truncated | Notch1 | Notch1 | N/A | human | synthetic |
| | with 2 residues | peptide, | CD8 hinge | | (Partial) | | Notch1NLS | zinc finger |
| | truncated | myc-tag, | | | | | | protein |
| | upstream of | anti-CD19 | | | | | | ZF6 |
| | RAM start, | scFv | | | | | | (SV40NLS) |
| | optimized | | | | | | | |
| | ZF6/Notch1NLS/p65- | | | | | | | |
| | MLXIPL SynTF | | | | | | | |
| pDP1761 | HingeNotch | CD8a signal | truncated | Notch1 | Notch1 | N/A | human | synthetic |
| | with 1 residue | peptide, | CD8 hinge | | (Partial) | | Notch1NLS | zinc finger |
| | truncated | myc-tag, | | | | | | protein |
| | upstream of | anti-CD19 | | | | | | ZF6 |
| | RAM start, | scFv | | | | | | (SV40NLS) |
| | optimized | | | | | | | |
| | ZF6/Notch1NLS/p65- | | | | | | | |
| | MLXIPL SynTF | | | | | | | |
| pDP1762 | HingeNotch | CD8a signal | truncated | Notch1 | Notch1 | human | human | synthetic |
| | with 1 RAM | peptide, | CD8 hinge | | | Notch1 | Notch1NLS | zinc finger |
| | residue, | myc-tag, | | | | RAM | | protein |
| | optimized | anti-CD19 | | | | (1/11) | | ZF6 |
| | ZF6/Notch1NLS/p65- | scFv | | | | | | (SV40NLS) |
| | MLXIPL SynTF | | | | | | | |
| pDP1763 | HingeNotch | CD8a signal | truncated | Notch1 | Notch1 | human | human | synthetic |
| | with 2 RAM | peptide, | CD8 hinge | | | Notch1 | Notch1NLS | zinc finger |
| | residues, | myc-tag, | | | | RAM | | protein |
| | optimized | anti-CD19 | | | | (2/11) | | ZF6 |
| | ZF6/Notch1NLS/p65- | scFv | | | | | | (SV40NLS) |
| | MLXIPL SynTF | | | | | | | |
| pDP1764 | HingeNotch | CD8a signal | truncated | Notch1 | Notch1 | human | human | synthetic |
| | with 3 RAM | peptide, | CD8 hinge | | | Notch1 | Notch1NLS | zinc finger |
| | residues, | myc-tag, | | | | RAM | | protein |
| | optimized | anti-CD19 | | | | (3/11) | | ZF6 |
| | ZF6/Notch1NLS/p65- | scFv | | | | | | (SV40NLS) |
| | MLXIPL SynTF | | | | | | | |
| pDP1765 | HingeNotch | CD8a signal | truncated | Notch1 | Notch1 | human | human | synthetic |
| | with 4 RAM | peptide, | CD8 hinge | | | Notch1 | Notch1NLS | zinc finger |
| | residues, | myc-tag, | | | | RAM | | protein |
| | optimized | anti-CD19 | | | | (4/11) | | ZF6 |
| | ZF6/Notch1NLS/p65- | scFv | | | | | | (SV40NLS) |
| | MLXIPL SynTF | | | | | | | |
| pDP1766 | HingeNotch | CD8a signal | truncated | Notch1 | Notch1 | human | human | synthetic |
| | with 5 RAM | peptide, | CD8 hinge | | | Notch1 | Notch1NLS | zinc finger |
| | residues, | myc-tag, | | | | RAM | | protein |
| | optimized | anti-CD19 | | | | (5/11) | | ZF6 |
| | ZF6/Notch1NLS/p65- | scFv | | | | | | (SV40NLS) |
| | MLXIPL SynTF | | | | | | | |
| pDP1767 | HingeNotch | CD8a signal | truncated | Notch1 | Notch1 | human | human | synthetic |
| | with 6 RAM | peptide, | CD8 hinge | | | Notch1 | Notch1NLS | zinc finger |
| | residues, | myc-tag, | | | | RAM | | protein |
| | optimized | anti-CD19 | | | | (6/11) | | ZF6 |
| | ZF6/Notch1NLS/p65- | scFv | | | | | | (SV40NLS) |
| | MLXIPL SynTF | | | | | | | |
| pDE1768 | HingeNotch | CD8a signal | truncated | Notch1 | Notch1 | human | human | synthetic |
| | with 7 RAM | peptide, | CD8 hinge | | | Notch1 | Notch1NLS | zinc finger |
| | residues, | myc-tag, | | | | RAM | | protein |
| | optimized | anti-CD19 | | | | (7/11) | | ZF6 |
| | ZF6/Notch1NLS/p65- | scFv | | | | | | (SV40NLS) |
| | MLXIPL SynTF | | | | | | | |
| pDP1725 | HingeNotch | CD8a signal | truncated | Notch1 | Notch1 | human | human | synthetic |
| | with 8 RAM | peptide, | CD8 hinge | | | Notch1 | Notch1NLS | zinc finger |
| | residues, | myc-tag, | | | | RAM | | protein |
| | optimized | anti-CD19 | | | | (8/11) | | ZF6 |
| | ZF6/Notch1NLS/p65- | scFv | | | | | | (SV40NLS) |
| | MLXIPL SynTF | | | | | | | |
| pDP1769 | HingeNotch | CD8a signal | truncated | Notch1 | Notch1 | human | human | synthetic |
| | with 9 RAM | peptide, | CD8 hinge | | | Notch1 | Notch1NLS | zinc finger |
| | residues, | myc-tag, | | | | RAM | | protein |
| | optimized | anti-CD19 | | | | (9/11) | | ZF6 |
| | ZF6/Notch1NLS/p65- | scFv | | | | | | (SV40NLS) |
| | MLXIPL SynTF | | | | | | | |
| pDP1770 | HingeNotch | CD8a signal | truncated | Notch1 | Notch1 | human | human | synthetic |
| | with 10 RAM | peptide, | CD8 hinge | | | Notch1 | Notch1NLS | zinc finger |
| | residues, | myc-tag, | | | | RAM | | protein |
| | optimized | anti-CD19 | | | | (10/11) | | ZF6 |
| | ZF6/Notch1NLS/p65- | scFv | | | | | | (SV40NLS) |
| | MLXIPL SynTF | | | | | | | |
| pDP1771 | HingeNotch | CD8a signal | truncated | Notch1 | Notch1 | human | human | synthetic |
| | with 11 RAM | peptide, | CD8 hinge | | | Notch1 | Notch1NLS | zinc finger |
| | residues, | myc-tag, | | | | RAM | | protein |
| | optimized | anti-CD19 | | | | (11/11) | | ZF6 |
| | ZF6/Notch1NLS/p65- | scFv | | | | | | (SV40NLS) |
| | MLXIPL SynTF | | | | | | | |
| pDP1441 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65 | anti-CD19 | | | | | | ZF6 |
| | SynTF | scFv | | | | | | (SV40NLS) |
| pDP1487 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65- | anti-CD19 | | | | | | ZF6 |
| | GR SynTF | scFv | | | | | | (SV40NLS) |
| pDP1558 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65- | anti-CD19 | | | | | | ZF6 |
| | MIER3 | scFv | | | | | | (SV40NLS) |
| | SynTF | | | | | | | |
| pDP1560 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65- | anti-CD19 | | | | | | ZF6 |
| | MLXIPL | scFv | | | | | | (SV40NLS) |
| | SynTF | | | | | | | |
| pDP1562 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65- | anti-CD19 | | | | | | ZF6 |
| | NFE2L1 | scFv | | | | | | (SV40NLS) |
| | SynTF | | | | | | | |
| pDP1566 | HingeNotch | CD8a signal | truncated | Notch1 | Notch2 | N/A | SV40NLS | synthetic |
| | (STS2) with | peptide, | CD8 hinge | | | | | zinc finger |
| | optimized | myc-tag, | | | | | | protein |
| | ZF6/SV40NLS/p65- | anti-CD19 | | | | | | ZF6 |
| | PTF1A | scFv | | | | | | (SV40NLS) |
| | SynTF | | | | | | | |
| pDP1673 | HingeNotch | truncated | truncated | Notch1 | Notch1 | N/A | SV40NLS | synthetic |
| | (Notch1 STS), | CD8 hinge | CD8 hinge | | | | | zinc finger |
| | optimized | | | | | | | protein |
| | ZF6/SV40NLS/p65- | | | | | | | ZF6 |
| | MLXIPL | | | | | | | (SV40NLS) |
| | SynTF | | | | | | | |
| pDP1674 | HingeNotch | truncated | truncated | Notch1 | Notch1 | human | SV40NLS | synthetic |
| | (Notch1 STS), 8 | CD8 hinge | CD8 hinge | | | Notch1 | | zinc finger |
| | RAM residues, | | | | | RAM | | protein |
| | optimized | | | | | (8/11) | | ZF6 |
| | ZF6/SV40NLS/p65- | | | | | | | (SV40NLS) |
| | MLXIPL SynTF | | | | | | | |
| pDP1675 | HingeNotch | truncated | truncated | Notch1 | Notch2 | human | SV40NLS | synthetic |
| | (Notch2 STS), 8 | CD8 hinge | CD8 hinge | | | Notch1 | | zinc finger |
| | RAM residues, | | | | | RAM | | protein |
| | optimized | | | | | (8/11) | | ZF6 |
| | ZF6/SV40NLS/p65- | | | | | | | (SV40NLS) |
| | MLXIPL SynTF | | | | | | | |
| pDP1757 | HingeNotch | CD8a signal | truncated | Notch1 | N/A | N/A | human | synthetic |
| | with 5 residues | peptide, | CD8 hinge | | | | Notch1NLS | zinc finger |
| | truncated | myc-tag, | | | | | | protein |
| | upstream of | anti-CD19 | | | | | | ZF6 |
| | RAM start, | scFv | | | | | | (SV40NLS) |
| | optimized | | | | | | | |
| | ZF6/Notch1NLS/p65- | | | | | | | |
| | MLXIPL SynTF | | | | | | | |
| pDP1758 | HingeNotch | CD8a signal | truncated | Notch1 | Notch1 | N/A | human | synthetic |
| | with 4 residues | peptide, | CD8 hinge | | (Partial) | | Notch1NLS | zinc finger |
| | truncated | myc-tag, | | | | | | protein |
| | upstream of | anti-CD19 | | | | | | ZF6 |
| | RAM start, | scFv | | | | | | (SV40NLS) |
| | optimized | | | | | | | |
| | ZF6/Notch1NLS/p65- | | | | | | | |
| | MLXIPL SynTF | | | | | | | |
| pDP1759 | HingeNotch | CD8a signal | truncated | Notch1 | Notch1 | N/A | human | synthetic |
| | with 3 residues | peptide, | CD8 hinge | | (Partial) | | Notch1NLS | zinc finger |
| | truncated | myc-tag, | | | | | | protein |
| | upstream of | anti-CD19 | | | | | | ZF6 |
| | RAM start, | scFv | | | | | | (SV40NLS) |
| | optimized | | | | | | | |
| | ZF6/Notch1NLS/p65- | | | | | | | |
| | MLXIPL SynTF | | | | | | | |

| Plasmid | Receptor | | | | |
|---|---|---|---|---|---|
| ID | description | Linker | TAD | Linker | TAD |
| pDP1441 | HingeNotch | N/A | human | N/A | N/A |
| | (STS2) with | | p65 | | |
| | optimized | | TAD | | |
| | ZF6/SV40NLS/p65 | | | | |
| | SynTF | | | | |
| pDP1487 | HingeNotch | N/A | human | N/A | human |
| | (STS2) with | | p65 | | GR |
| | optimized | | TAD | | TAD1 |
| | ZF6/SV40NLS/p65- | | | | |
| | GR SynTF | | | | |
| pDP1558 | HingeNotch | N/A | human | N/A | human |
| | (STS2) with | | p65 | | MIER3 |
| | optimized | | TAD | | TAD |
| | ZF6/SV40NLS/p65- | | | | fragmnet |
| | MIER3 | | | | |
| | SynTF | | | | |
| pDP1560 | HingeNotch | N/A | human | N/A | human |
| | (STS2) with | | p65 | | MLXIPL |
| | optimized | | TAD | | TAD |
| | ZF6/SV40NLS/p65- | | | | fragmnet |
| | MLXIPL | | | | |
| | SynTF | | | | |
| pDP1562 | HingeNotch | N/A | human | N/A | human |
| | (STS2) with | | p65 | | NFE2L1 |
| | optimized | | TAD | | TAD |
| | ZF6/SV40NLS/p65- | | | | fragmnet |
| | NFE2L1 | | | | |
| | SynTF | | | | |
| pDP1566 | HingeNotch | N/A | human | N/A | human |
| | (STS2) with | | p65 | | PTF1A |
| | optimized | | TAD | | TAD |
| | ZF6/SV40NLS/p65- | | | | fragmnet |
| | PTF1A | | | | |
| | SynTF | | | | |
| pDP1673 | HingeNotch | N/A | human | N/A | human |
| | (Notch1 STS), | | p65 | | MLXIPL |
| | optimized | | TAD | | TAD |
| | ZF6/SV40NLS/p65- | | | | fragmnet |
| | MLXIPL | | | | |
| | SynTF | | | | |
| pDP1674 | HingeNotch | N/A | human | N/A | human |
| | (Notch1 STS), 8 | | p65 | | MLXIPL |
| | RAM residues, | | TAD | | TAD |
| | optimized | | | | fragmnet |
| | ZF6/SV40NLS/p65- | | | | |
| | MLXIPL SynTF | | | | |
| pDP1675 | HingeNotch | N/A | human | N/A | human |
| | (Notch2 STS), 8 | | p65 | | MLXIPL |
| | RAM residues, | | TAD | | TAD |
| | optimized | | | | fragmnet |
| | ZF6/SV40NLS/p65- | | | | |
| | MLXIPL SynTF | | | | |
| pDP1757 | HingeNotch | N/A | human | N/A | human |
| | with 5 residues | | p65 | | MLXIPL |
| | truncated | | TAD | | TAD |
| | upstream of | | | | fragmnet |
| | RAM start, | | | | |
| | optimized | | | | |
| | ZF6/Notch1NLS/p65- | | | | |
| | MLXIPL SynTF | | | | |
| pDP1758 | HingeNotch | N/A | human | N/A | human |
| | with 4 residues | | p65 | | MLXIPL |
| | truncated | | TAD | | TAD |
| | upstream of | | | | fragmnet |
| | RAM start, | | | | |
| | optimized | | | | |
| | ZF6/Notch1NLS/p65- | | | | |
| | MLXIPL SynTF | | | | |
| pDP1759 | HingeNotch | N/A | human | N/A | human |
| | with 3 residues | | p65 | | MLXIPL |
| | truncated | | TAD | | TAD |
| | upstream of | | | | fragmnet |
| | RAM start, | | | | |
| | optimized | | | | |
| | ZF6/Notch1NLS/p65- | | | | |
| | MLXIPL SynTF | | | | |
| pDP1760 | HingeNotch | N/A | human | N/A | human |
| | with 2 residues | | p65 | | MLXIPL |
| | truncated | | TAD | | TAD |
| | upstream of | | | | fragmnet |
| | RAM start, | | | | |
| | optimized | | | | |
| | ZF6/Notch1NLS/p65- | | | | |
| | MLXIPL SynTF | | | | |
| pDP1761 | HingeNotch | N/A | human | N/A | human |
| | with 1 residue | | p65 | | MLXIPL |
| | truncated | | TAD | | TAD |
| | upstream of | | | | fragmnet |
| | RAM start, | | | | |
| | optimized | | | | |
| | ZF6/Notch1NLS/p65- | | | | |
| | MLXIPL SynTF | | | | |
| pDP1762 | HingeNotch | N/A | human | N/A | human |
| | with 1 RAM | | p65 | | MLXIPL |
| | residue, | | TAD | | TAD |
| | optimized | | | | fragmnet |
| | ZF6/Notch1NLS/p65- | | | | |
| | MLXIPL SynTF | | | | |
| pDP1763 | HingeNotch | N/A | human | N/A | human |
| | with 2 RAM | | p65 | | MLXIPL |
| | residues, | | TAD | | TAD |
| | optimized | | | | fragmnet |
| | ZF6/Notch1NLS/p65- | | | | |
| | MLXIPL SynTF | | | | |
| pDP1764 | HingeNotch | N/A | human | N/A | human |
| | with 3 RAM | | p65 | | MLXIPL |
| | residues, | | TAD | | TAD |
| | optimized | | | | fragmnet |
| | ZF6/Notch1NLS/p65- | | | | |
| | MLXIPL SynTF | | | | |
| pDP1765 | HingeNotch | N/A | human | N/A | human |
| | with 4 RAM | | p65 | | MLXIPL |
| | residues, | | TAD | | TAD |
| | optimized | | | | fragmnet |
| | ZF6/Notch1NLS/p65- | | | | |
| | MLXIPL SynTF | | | | |
| pDP1766 | HingeNotch | N/A | human | N/A | human |
| | with 5 RAM | | p65 | | MLXIPL |
| | residues, | | TAD | | TAD |
| | optimized | | | | fragmnet |
| | ZF6/Notch1NLS/p65- | | | | |
| | MLXIPL SynTF | | | | |
| pDP1767 | HingeNotch | N/A | human | N/A | human |
| | with 6 RAM | | p65 | | MLXIPL |
| | residues, | | TAD | | TAD |
| | optimized | | | | fragmnet |
| | ZF6/Notch1NLS/p65- | | | | |
| | MLXIPL SynTF | | | | |
| pDP1768 | HingeNotch | N/A | human | N/A | human |
| | with 7 RAM | | p65 | | MLXIPL |
| | residues, | | TAD | | TAD |
| | optimized | | | | fragmnet |
| | ZF6/Notch1NLS/p65- | | | | |
| | MLXIPL SynTF | | | | |
| pDP1725 | HingeNotch | N/A | human | N/A | human |
| | with 8 RAM | | p65 | | MLXIPL |
| | residues, | | TAD | | TAD |
| | optimized | | | | fragmnet |
| | ZF6/Notch1NLS/p65- | | | | |
| | MLXIPL SynTF | | | | |
| pDP1769 | HingeNotch | N/A | human | N/A | human |
| | with 9 RAM | | p65 | | MLXIPL |
| | residues, | | TAD | | TAD |
| | optimized | | | | fragmnet |
| | ZF6/Notch1NLS/p65- | | | | |
| | MLXIPL SynTF | | | | |
| pDP1770 | HingeNotch | N/A | human | N/A | human |
| | with 10 RAM | | p65 | | MLXIPL |
| | residues, | | TAD | | TAD |
| | optimized | | | | fragmnet |
| | ZF6/Notch1NLS/p65- | | | | |
| | MLXIPL SynTF | | | | |
| pDP1771 | HingeNotch | N/A | human | N/A | human |
| | with 11 RAM | | p65 | | MLXIPL |
| | residues, | | TAD | | TAD |
| | optimized | | | | fragmnet |
| | ZF6/Notch1NLS/p65- | | | | |
| | MLXIPL SynTF | | | | |
| pDP1441 | HingeNotch | N/A | human | N/A | N/A |
| | (STS2) with | | p65 | | |
| | optimized | | TAD | | |
| | ZF6/SV40NLS/p65 | | | | |
| | SynTF | | | | |
| pDP1487 | HingeNotch | N/A | human | N/A | human |
| | (STS2) with | | p65 | | GR |
| | optimized | | TAD | | TAD1 |
| | ZF6/SV40NLS/p65- | | | | |
| | GR SynTF | | | | |
| pDP1558 | HingeNotch | N/A | human | N/A | human |
| | (STS2) with | | p65 | | MIER3 |
| | optimized | | TAD | | TAD |
| | ZF6/SV40NLS/p65- | | | | fragmnet |
| | MIER3 | | | | |
| | SynTF | | | | |
| pDP1560 | HingeNotch | N/A | human | N/A | human |
| | (STS2) with | | p65 | | MLXIPL |
| | optimized | | TAD | | TAD |
| | ZF6/SV40NLS/p65- | | | | fragmnet |
| | MLXIPL | | | | |
| | SynTF | | | | |
| pDP1562 | HingeNotch | N/A | human | N/A | human |
| | (STS2) with | | p65 | | NFE2L1 |
| | optimized | | TAD | | TAD |
| | ZF6/SV40NLS/p65- | | | | fragmnet |
| | NFE2L1 | | | | |
| | SynTF | | | | |
| pDP1566 | HingeNotch | N/A | human | N/A | human |
| | (STS2) with | | p65 | | PTF1A |
| | optimized | | TAD | | TAD |
| | ZF6/SV40NLS/p65- | | | | fragmnet |
| | PTF1A | | | | |
| | SynTF | | | | |
| pDP1673 | HingeNotch | N/A | human | N/A | human |
| | (Notch1 STS), | | p65 | | MLXIPL |
| | optimized | | TAD | | TAD |
| | ZF6/SV40NLS/p65- | | | | fragmnet |
| | MLXIPL | | | | |
| | SynTF | | | | |
| pDP1674 | HingeNotch | N/A | human | N/A | human |
| | (Notch1 STS), 8 | | p65 | | MLXIPL |
| | RAM residues, | | TAD | | TAD |
| | optimized | | | | fragmnet |
| | ZF6/SV40NLS/p65- | | | | |
| | MLXIPL SynTF | | | | |
| pDP1675 | HingeNotch | N/A | human | N/A | human |
| | (Notch2 STS), 8 | | p65 | | MLXIPL |
| | RAM residues, | | TAD | | TAD |
| | optimized | | | | fragmnet |
| | ZF6/SV40NLS/p65- | | | | |
| | MLXIPL SynTF | | | | |
| pDP1757 | HingeNotch | N/A | human | N/A | human |
| | with 5 residues | | p65 | | MLXIPL |
| | truncated | | TAD | | TAD |
| | upstream of | | | | fragmnet |
| | RAM start, | | | | |
| | optimized | | | | |
| | ZF6/Notch1NLS/p65- | | | | |
| | MLXIPL SynTF | | | | |
| pDP1758 | HingeNotch | N/A | human | N/A | human |
| | with 4 residues | | p65 | | MLXIPL |
| | truncated | | TAD | | TAD |
| | upstream of | | | | fragmnet |
| | RAM start, | | | | |
| | optimized | | | | |
| | ZF6/Notch1NLS/p65- | | | | |
| | MLXIPL SynTF | | | | |
| pDP1759 | HingeNotch | N/A | human | N/A | human |
| | with 3 residues | | p65 | | MLXIPL |
| | truncated | | TAD | | TAD |
| | upstream of | | | | fragmnet |
| | RAM start, | | | | |
| | optimized | | | | |
| | ZF6/Notch1NLS/p65- | | | | |
| | MLXIPL SynTF | | | | |

In addition, the reference sequences from which each of the components listed in Table 4 is derived from are listed in Table 5 below.

TABLE 5
sequence references for human or humanized HingeNotch
receptors with augmented transactivation domain.

| Plasmid | Receptor | | | |
|---|---|---|---|---|
| ID | description | ECD | N-JMD | TMD |
| pDP1441 | HingeNotch | Uniprot | Uniprot | Uniprot |
| | (STS2) with | P01732: | P46531: | P46531: |
| | optimized | AA1-21, | AA1427- | AA1736- |
| | ZF6/SV40NLS/p65 | Uniprot | 1447, | 1757 |
| | SynTF | P01106: | AA1725- | |
| | | AA410-419, | 1735 | |
| | | FMC63 | | |
| | | scFv | | |
| | | sequence | | |
| | | (https://www.ncbi.nlm.nih.gov/pubmed/9566763/) | | |
| pDP1487 | HingeNotch | Uniprot | Uniprot | Uniprot |
| | (STS2) with | P01732: | P46531: | P46531: |
| | optimized | AA1-21, | AA1427- | AA1736- |
| | ZF6/SV40NLS/p65- | Uniprot | 1447, | 1757 |
| | GR SynTF | P01106: | AA1725- | |
| | | AA410-419, | 1735 | |
| | | FMC63 | | |
| | | scFv | | |
| | | sequence | | |
| | | (https://www.ncbi.nlm.nih.gov/pubmed/9566763/) | | |
| pDP1558 | HingeNotch | Uniprot | Uniprot | Uniprot |
| | (STS2) with | P01732: | P46531: | P46531: |
| | optimized | AA1-21, | AA1427- | AA1736- |
| | ZF6/SV40NLS/p65- | Uniprot | 1447, | 1757 |
| | MIER3 | P01106: | AA1725- | |
| | SynTF | AA410-419, | 1735 | |
| | | FMC63 | | |
| | | scFv | | |
| | | sequence | | |
| | | (https://www.ncbi.nlm.nih.gov/pubmed/9566763/) | | |
| pDP1560 | HingeNotch | Uniprot | Uniprot | Uniprot |
| | (STS2) with | P01732: | P46531: | P46531: |
| | optimized | AA1-21, | AA1427- | AA1736- |
| | ZF6/SV40NLS/p65- | Uniprot | 1447, | 1757 |
| | MLXIPL | P01106: | AA1725- | |
| | SynTF | AA410-419, | 1735 | |
| | | FMC63 | | |
| | | scFv | | |
| | | sequence | | |
| | | (https://www.ncbi.nlm.nih.gov/pubmed/9566763/) | | |
| pDP1562 | HingeNotch | Uniprot | Uniprot | Uniprot |
| | (STS2) with | P01732: | P46531: | P46531: |
| | optimized | AA1-21, | AA1427- | AA1736- |
| | ZF6/SV40NLS/p65- | Uniprot | 1447, | 1757 |
| | NFE2L1 | P01106: | AA1725- | |
| | SynTF | AA410-419, | 1735 | |
| | | FMC63 | | |
| | | scFv | | |
| | | sequence | | |
| | | (https://www.ncbi.nlm.nih.gov/pubmed/9566763/) | | |
| pDP1566 | HingeNotch | Uniprot | Uniprot | Uniprot |
| | (STS2) with | P01732: | P46531: | P46531: |
| | optimized | AA1-21, | AA1427- | AA1736- |
| | ZF6/SV40NLS/p65- | Uniprot | 1447, | 1757 |
| | PTF1A | P01106: | AA1725- | |
| | SynTF | AA410-419, | 1735 | |
| | | FMC63 | | |
| | | scFv | | |
| | | sequence | | |
| | | (https://www.ncbi.nlm.nih.gov/pubmed/9566763/) | | |
| pDP1673 | HingeNotch | Uniprot | Uniprot | Uniprot |
| | (Notch1 | P01732: | P46531: | P46531: |
| | STS), | AA1-21, | AA1427- | AA1736- |
| | optimized | Uniprot | 1447, | 1757 |
| | ZF6/SV40NLS/p65- | P01106: | AA1725- | |
| | MLXIPL | AA410-419, | 1735 | |
| | SynTF | FMC63 | | |
| | | scFv | | |
| | | sequence | | |
| | | (https://www.ncbi.nlm.nih.gov/pubmed/9566763/) | | |
| pDP1674 | HingeNotch | Uniprot | Uniprot | Uniprot |
| | (Notch1 | P01732: | P46531: | P46531: |
| | STS), 8 | AA1-21, | AA1427- | AA1736- |
| | RAM | Uniprot | 1447, | 1757 |
| | residues, | P01106: | AA1725- | |
| | optimized | AA410-419, | 1735 | |
| | ZF6/SV40NLS/p65- | FMC63 | | |
| | MLXIPL | scFv | | |
| | SynTF | sequence | | |
| | | (https://www.ncbi.nlm.nih.gov/pubmed/9566763/) | | |
| pDP1675 | HingeNotch | Uniprot | Uniprot | Uniprot |
| | (Notch2 | P01732: | P46531: | P46531: |
| | STS), 8 | AA1-21, | AA1427- | AA1736- |
| | RAM | Uniprot | 1447, | 1757 |
| | residues, | P01106: | AA1725- | |
| | optimized | AA410-419, | 1735 | |
| | ZF6/SV40NLS/p65- | FMC63 | | |
| | MLXIPL | scFv | | |
| | SynTF | sequence | | |
| | | (https://www.ncbi.nlm.nih.gov/pubmed/9566763/) | | |
| pDP1757 | HingeNotch | Uniprot | Uniprot | Uniprot |
| | with 5 | P01732: | P46531: | P46531: |
| | residues | AA1-21, | AA1427- | AA1736- |
| | truncated | Uniprot | 1447, | 1757 |
| | upstream of | P01106: | AA1725- | |
| | RAM start, | AA410-419, | 1735 | |
| | optimized | FMC63 | | |
| | ZF6/Notch1NLS/p65- | scFv | | |
| | MLXIPL | sequence | | |
| | SynTF | (https://www.ncbi.nlm.nih.gov/pubmed/9566763/) | | |
| pDP1758 | HingeNotch | Uniprot | Uniprot | Uniprot |
| | with 4 | P01732: | P46531: | P46531: |
| | residues | AA1-21, | AA1427- | AA1736- |
| | truncated | Uniprot | 1447, | 1757 |
| | upstream of | P01106: | AA1725- | |
| | RAM start, | AA410-419, | 1735 | |
| | optimized | FMC63 | | |
| | ZF6/Notch1NLS/p65- | scFv | | |
| | MLXIPL | sequence | | |
| | SynTF | (https://www.ncbi.nlm.nih.gov/pubmed/9566763/) | | |
| pDP1759 | HingeNotch | Uniprot | Uniprot | Uniprot |
| | with 3 | P01732: | P46531: | P46531: |
| | residues | AA1-21, | AA1427- | AA1736- |
| | truncated | Uniprot | 1447, | 1757 |
| | upstream of | P01106: | AA1725- | |
| | RAM start, | AA410-419, | 1735 | |
| | optimized | FMC63 | | |
| | ZF6/Notch1NLS/p65- | scFv | | |
| | MLXIPL | sequence | | |
| | SynTF | (https://www.ncbi.nlm.nih.gov/pubmed/9566763/) | | |
| pDP1760 | HingeNotch | Uniprot | Uniprot | Uniprot |
| | with 2 | P01732: | P46531: | P46531: |
| | residues | AA1-21, | AA1427- | AA1736- |
| | truncated | Uniprot | 1447, | 1757 |
| | upstream of | P01106: | AA1725- | |
| | RAM start, | AA410-419, | 1735 | |
| | optimized | FMC63 | | |
| | ZF6/Notch1NLS/p65- | scFv | | |
| | MLXIPL | sequence | | |
| | SynTF | (https://www.ncbi.nlm.nih.gov/pubmed/9566763/) | | |
| pDP1761 | HingeNotch | Uniprot | Uniprot | Uniprot |
| | with 1 | P01732: | P46531: | P46531: |
| | residue | AA1-21, | AA1427- | AA1736- |
| | truncated | Uniprot | 1447, | 1757 |
| | upstream of | P01106: | AA1725- | |
| | RAM start, | AA410-419, | 1735 | |
| | optimized | FMC63 | | |
| | ZF6/Notch1NLS/p65- | scFv | | |
| | MLXIPL | sequence | | |
| | SynTF | (https://www.ncbi.nlm.nih.gov/pubmed/9566763/) | | |
| pDP1762 | HingeNotch | Uniprot | Uniprot | Uniprot |
| | with 1 RAM | P01732: | P46531: | P46531: |
| | residue, | AA1-21, | AA1427- | AA1736- |
| | optimized | Uniprot | 1447, | 1757 |
| | ZF6/Notch1NLS/p65- | P01106: | AA1725- | |
| | MLXIPL | AA410-419, | 1735 | |
| | SynTF | FMC63 | | |
| | | scFv | | |
| | | sequence | | |
| | | (https://www.ncbi.nlm.nih.gov/pubmed/9566763/) | | |
| pDP1763 | HingeNotch | Uniprot | Uniprot | Uniprot |
| | with 2 RAM | P01732: | P46531: | P46531: |
| | residues, | AA1-21, | AA1427- | AA1736- |
| | optimized | Uniprot | 1447, | 1757 |
| | ZF6/Notch1NLS/p65- | P01106: | AA1725- | |
| | MLXIPL | AA410-419, | 1735 | |
| | SynTF | FMC63 | | |
| | | scFv | | |
| | | sequence | | |
| | | (https://www.ncbi.nlm.nih.gov/pubmed/9566763/) | | |
| pDP1764 | HingeNotch | Uniprot | Uniprot | Uniprot |
| | with 3 RAM | P01732: | P46531: | P46531: |
| | residues, | AA1-21, | AA1427- | AA1736- |
| | optimized | Uniprot | 1447, | 1757 |
| | ZF6/Notch1NLS/p65- | P01106: | AA1725- | |
| | MLXIPL | AA410-419, | 1735 | |
| | SynTF | FMC63 | | |
| | | scFv | | |
| | | sequence | | |
| | | (https://www.ncbi.nlm.nih.gov/pubmed/9566763/) | | |
| pDP1765 | HingeNotch | Uniprot | Uniprot | Uniprot |
| | with 4 RAM | P01732: | P46531: | P46531: |
| | residues, | AA1-21, | AA1427- | AA1736- |
| | optimized | Uniprot | 1447, | 1757 |
| | ZF6/Notch1NLS/p65- | P01106: | AA1725- | |
| | MLXIPL | AA410-419, | 1735 | |
| | SynTF | FMC63 | | |
| | | scFv | | |
| | | sequence | | |
| | | (https://www.ncbi.nlm.nih.gov/pubmed/9566763/) | | |
| pDP1766 | HingeNotch | Uniprot | Uniprot | Uniprot |
| | with 5 RAM | P01732: | P46531: | P46531: |
| | residues, | AA1-21, | AA1427- | AA1736- |
| | optimized | Uniprot | 1447, | 1757 |
| | ZF6/Notch1NLS/p65- | P01106: | AA1725- | |
| | MLXIPL | AA410-419, | 1735 | |
| | SynTF | FMC63 | | |
| | | scFv | | |
| | | sequence | | |
| | | (https://www.ncbi.nlm.nih.gov/pubmed/9566763/) | | |
| pDP1767 | HingeNotch | Uniprot | Uniprot | Uniprot |
| | with 6 RAM | P01732: | P46531: | P46531: |
| | residues, | AA1-21, | AA1427- | AA1736- |
| | optimized | Uniprot | 1447, | 1757 |
| | ZF6/Notch1NLS/p65- | P01106: | AA1725- | |
| | MLXIPL | AA410-419, | 1735 | |
| | SynTF | FMC63 | | |
| | | scFv | | |
| | | sequence | | |
| | | (https://www.ncbi.nlm.nih.gov/pubmed/9566763/) | | |
| pDP1768 | HingeNotch | Uniprot | Uniprot | Uniprot |
| | with 7 RAM | P01732: | P46531: | P46531: |
| | residues, | AA1-21, | AA1427- | AA1736- |
| | optimized | Uniprot | 1447, | 1757 |
| | ZF6/Notch1NLS/p65- | P01106: | AA1725- | |
| | MLXIPL | AA410-419, | 1735 | |
| | SynTF | FMC63 | | |
| | | scFv | | |
| | | sequence | | |
| | | (https://www.ncbi.nlm.nih.gov/pubmed/9566763/) | | |
| pDP1725 | HingeNotch | Uniprot | Uniprot | Uniprot |
| | with 8 RAM | P01732: | P46531: | P46531: |
| | residues, | AA1-21, | AA1427- | AA1736- |
| | optimized | Uniprot | 1447, | 1757 |
| | ZF6/Notch1NLS/p65- | P01106: | AA1725- | |
| | MLXIPL | AA410-419, | 1735 | |
| | SynTF | FMC63 | | |
| | | scFv | | |
| | | sequence | | |
| | | (https://www.ncbi.nlm.nih.gov/pubmed/9566763/) | | |
| pDP1769 | HingeNotch | Uniprot | Uniprot | Uniprot |
| | with 9 RAM | P01732: | P46531: | P46531: |
| | residues, | AA1-21, | AA1427- | AA1736- |
| | optimized | Uniprot | 1447, | 1757 |
| | ZF6/Notch1NLS/p65- | P01106: | AA1725- | |
| | MLXIPL | AA410-419, | 1735 | |
| | SynTF | FMC63 | | |
| | | scFv | | |
| | | sequence | | |
| | | (https://www.ncbi.nlm.nih.gov/pubmed/9566763/) | | |
| pDP1770 | HingeNotch | Uniprot | Uniprot | Uniprot |
| | with 10 | P01732: | P46531: | P46531: |
| | RAM | AA1-21, | AA1427- | AA1736- |
| | residues, | Uniprot | 1447, | 1757 |
| | optimized | P01106: | AA1725- | |
| | ZF6/Notch1NLS/p65- | AA410-419, | 1735 | |
| | MLXIPL | FMC63 | | |
| | SynTF | scFv | | |
| | | sequence | | |
| | | (https://www.ncbi.nlm.nih.gov/pubmed/9566763/) | | |
| pDP1771 | HingeNotch | Uniprot | Uniprot | Uniprot |
| | with 11 | P01732: | P46531: | P46531: |
| | RAM | AA1-21, | AA1427- | AA1736- |
| | residues, | Uniprot | 1447, | 1757 |
| | optimized | P01106: | AA1725- | |
| | ZF6/Notch1NLS/p65- | AA410-419, | 1735 | |
| | MLXIPL | FMC63 | | |
| | SynTF | scFv | | |
| | | sequence | | |
| | | (https://www.ncbi.nlm.nih.gov/pubmed/9566763/) | | |

| Plasmid | | | |
|---|---|---|---|
| ID | STS | RAM | NLS |
| pDP1441 | Uniprot | N/A | Uniprot |
| | P46531: | | P03070 |
| | AA1758- | | AA |
| | 1762 | | 125-132 |
| pDP1487 | Uniprot | N/A | Uniprot |
| | P46531: | | P03070 |
| | AA1758- | | AA |
| | 1762 | | 125-132 |
| pDP1558 | Uniprot | N/A | Uniprot |
| | P46531: | | P03070 |
| | AA1758- | | AA |
| | 1762 | | 125-132 |
| pDP1560 | Uniprot | N/A | Uniprot |
| | P46531: | | P03070 |
| | AA1758- | | AA |
| | 1762 | | 125-132 |
| pDP1562 | Uniprot | N/A | Uniprot |
| | P46531: | | P03070 |
| | AA1758- | | AA |
| | 1762 | | 125-132 |
| pDP1566 | Uniprot | N/A | Uniprot |
| | P46531: | | P03070 |
| | AA1758- | | AA |
| | 1762 | | 125-132 |
| pDP1673 | NCBI | N/A | Uniprot |
| | NP_{—} | | P03070 |
| | 060087 | | AA |
| | AA | | 125-132 |
| | 1758- | | |
| | 1762 | | |
| pDP1674 | NCBI | NCBI | Uniprot |
| | NP_{—} | NP_{—} | P03070 |
| | 060087 | 060087 | AA |
| | AA | AA | 125-132 |
| | 1758- | 1763- | |
| | 1762 | 1770 | |
| pDP1675 | Uniprot | NCBI | Uniprot |
| | P46531: | NP_{—} | P03070 |
| | AA1758- | 060087 | AA |
| | 1762 | AA | 125-132 |
| | | 1763- | |
| | | 1770 | |
| pDP1757 | N/A | N/A | NCBI |
| | | | NP_{—} |
| | | | 060087 |
| | | | AA |
| | | | 2179- |
| | | | 2182 |
| pDP1758 | NCBI | N/A | NCBI |
| | NP_{—} | | NP_{—} |
| | 060087 | | 060087 |
| | AA | | AA |
| | 1758- | | 2179- |
| | 1758 | | 2182 |
| pDP1759 | NCBI | N/A | NCBI |
| | NP_{—} | | NP_{—} |
| | 060087 | | 060087 |
| | AA | | AA |
| | 1758- | | 2179- |
| | 1759 | | 2182 |
| pDP1760 | NCBI | N/A | NCBI |
| | NP_{—} | | NP_{—} |
| | 060087 | | 060087 |
| | AA | | AA |
| | 1758- | | 2179- |
| | 1760 | | 2182 |
| pDP1761 | NCBI | N/A | NCBI |
| | NP_{—} | | NP_{—} |
| | 060087 | | 060087 |
| | AA | | AA |
| | 1758- | | 2179- |
| | 1761 | | 2182 |
| pDP1762 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1763 | 2182 |
| pDP1763 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1764 | 2182 |
| pDP1764 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1765 | 2182 |
| pDP1765 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1766 | 2182 |
| pDP1766 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1767 | 2182 |
| pDP1767 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1768 | 2182 |
| pDP1768 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1769 | 2182 |
| pDP1725 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1770 | 2182 |
| pDP1769 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1771 | 2182 |
| pDP1770 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1772 | 2182 |
| pDP1771 | NCBI | NCBI | NCBI |
| | NP_{—} | NP_{—} | NP_{—} |
| | 060087 | 060087 | 060087 |
| | AA | AA | AA |
| | 1758- | 1763- | 2179- |
| | 1762 | 1773 | 2182 |

| Plasmid | Receptor | | | | | |
|---|---|---|---|---|---|---|
| ID | description | TF | Linker | TAD | Linker | TAD |
| pDP1441 | HingeNotch | Reference | N/A | UNIPROT | N/A | N/A |
| | (STS2) with | synthetic | | Q04206: | | |
| | optimized | zinc finger | | AA 361- | | |
| | ZF6/SV40NLS/p65 | protein | | 551 | | |
| | SynTF | | | | | |
| pDP1487 | HingeNotch | Reference | N/A | UNIPROT | N/A | UNIPROT |
| | (STS2) with | synthetic | | Q04206: | | P04150: |
| | optimized | zinc finger | | AA 361- | | AA 187- |
| | ZF6/SV40NLS/p65-GR | protein | | 551 | | 244 |
| | SynTF | | | | | |
| pDP1558 | HingeNotch | Reference | N/A | UNIPROT | N/A | NCBI |
| | (STS2) with | synthetic | | Q04206: | | NP_{—} |
| | optimized | zinc finger | | AA 361- | | 001284527.1 |
| | ZF6/SV40NLS/p65- | protein | | 551 | | AA |
| | MIER3 | | | | | 37-89 |
| | SynTF | | | | | |
| pDP1560 | HingeNotch | Reference | N/A | UNIPROT | N/A | NCBI |
| | (STS2) with | synthetic | | Q04206: | | XP_{—} |
| | optimized | zinc finger | | AA 361- | | 011514581.1 |
| | ZF6/SV40NLS/p65- | protein | | 551 | | AA |
| | MLXIPL | | | | | 318-370 |
| | SynTF | | | | | |
| pDP1562 | HingeNotch | Reference | N/A | UNIPROT | N/A | NCBI |
| | (STS2) with | synthetic | | Q04206: | | AAI31770.1 |
| | optimized | zinc finger | | AA 361- | | AA 141- |
| | ZF6/SV40NLS/p65- | protein | | 551 | | 193 |
| | NFE2L1 | | | | | |
| | SynTF | | | | | |
| pDP1566 | HingeNotch | Reference | N/A | UNIPROT | N/A | NCBI |
| | (STS2) with | synthetic | | Q04206: | | NP_{—} |
| | optimized | zinc finger | | AA 361- | | 835455 |
| | ZF6/SV40NLS/p65- | protein | | 551 | | AA |
| | PTF1A | | | | | 2-54 |
| | SynTF | | | | | |
| pDP1673 | HingeNotch | Reference | N/A | UNIPROT | N/A | NCBI |
| | (Notch1 | synthetic | | Q04206: | | XP_{—} |
| | STS), | zinc finger | | AA 361- | | 011514581.1 |
| | optimized | protein | | 551 | | AA |
| | ZF6/SV40NLS/p65- | | | | | 318-370 |
| | MLXIPL | | | | | |
| | SynTF | | | | | |
| pDP1674 | HingeNotch | Reference | N/A | UNIPROT | N/A | NCBI |
| | (Notch1 | synthetic | | Q04206: | | XP_{—} |
| | STS), 8 | zinc finger | | AA 361- | | 011514581.1 |
| | RAM | protein | | 551 | | AA |
| | residues, | | | | | 318-370 |
| | optimized | | | | | |
| | ZF6/SV40NLS/p65- | | | | | |
| | MLXIPL | | | | | |
| | SynTF | | | | | |
| pDP1675 | HingeNotch | Reference | N/A | UNIPROT | N/A | NCBI |
| | (Notch2 | synthetic | | Q04206: | | XP_{—} |
| | STS), 8 | zinc finger | | AA 361- | | 011514581.1 |
| | RAM | protein | | 551 | | AA |
| | residues, | | | | | 318-370 |
| | optimized | | | | | |
| | ZF6/SV40NLS/p65- | | | | | |
| | MLXIPL | | | | | |
| | SynTF | | | | | |
| pDP1757 | HingeNotch | Reference | N/A | UNIPROT | N/A | NCBI |
| | with 5 | synthetic | | Q04206: | | XP_{—} |
| | residues | zinc finger | | AA 361- | | 011514581.1 |
| | truncated | protein | | 551 | | AA |
| | upstream of | | | | | 318-370 |
| | RAM start, | | | | | |
| | optimized | | | | | |
| | ZF6/Notch1NLS/p65- | | | | | |
| | MLXIPL | | | | | |
| | SynTF | | | | | |
| pDP1758 | HingeNotch | Reference | N/A | UNIPROT | N/A | NCBI |
| | with 4 | synthetic | | Q04206: | | XP_{—} |
| | residues | zinc finger | | AA 361- | | 011514581.1 |
| | truncated | protein | | 551 | | AA |
| | upstream of | | | | | 318-370 |
| | RAM start, | | | | | |
| | optimized | | | | | |
| | ZF6/Notch1NLS/p65- | | | | | |
| | MLXIPL | | | | | |
| | SynTF | | | | | |
| pDP1759 | HingeNotch | Reference | N/A | UNIPROT | N/A | NCBI |
| | with 3 | synthetic | | Q04206: | | XP_{—} |
| | residues | zinc finger | | AA 361- | | 011514581.1 |
| | truncated | protein | | 551 | | AA |
| | upstream of | | | | | 318-370 |
| | RAM start, | | | | | |
| | optimized | | | | | |
| | ZF6/Notch1NLS/p65- | | | | | |
| | MLXIPL | | | | | |
| | SynTF | | | | | |
| pDP1760 | HingeNotch | Reference | N/A | UNIPROT | N/A | NCBI |
| | with 2 | synthetic | | Q04206: | | XP_{—} |
| | residues | zinc finger | | AA 361- | | 011514581.1 |
| | truncated | protein | | 551 | | AA |
| | upstream of | | | | | 318-370 |
| | RAM start, | | | | | |
| | optimized | | | | | |
| | ZF6/Notch1NLS/p65- | | | | | |
| | MLXIPL | | | | | |
| | SynTF | | | | | |
| pDP1761 | HingeNotch | Reference | N/A | UNIPROT | N/A | NCBI |
| | with 1 | synthetic | | Q04206: | | XP_{—} |
| | residue | zinc finger | | AA 361- | | 011514581.1 |
| | truncated | protein | | 551 | | AA |
| | upstream of | | | | | 318-370 |
| | RAM start, | | | | | |
| | optimized | | | | | |
| | ZF6/Notch1NLS/p65- | | | | | |
| | MLXIPL | | | | | |
| | SynTF | | | | | |
| pDP1762 | HingeNotch | Reference | N/A | UNIPROT | N/A | NCBI |
| | with 1 RAM | synthetic | | Q04206: | | XP_{—} |
| | residue, | zinc finger | | AA 361- | | 011514581.1 |
| | optimized | protein | | 551 | | AA |
| | ZF6/Notch1NLS/p65- | | | | | 318-370 |
| | MLXIPL | | | | | |
| | SynTF | | | | | |
| pDP1763 | HingeNotch | Reference | N/A | UNIPROT | N/A | NCBI |
| | with 2 RAM | synthetic | | Q04206: | | XP_{—} |
| | residues, | zinc finger | | AA 361- | | 011514581.1 |
| | optimized | protein | | 551 | | AA |
| | ZF6/Notch1NLS/p65- | | | | | 318-370 |
| | MLXIPL | | | | | |
| | SynTF | | | | | |
| pDP1764 | HingeNotch | Reference | N/A | UNIPROT | N/A | NCBI |
| | with 3 RAM | synthetic | | Q04206: | | XP_{—} |
| | residues, | zinc finger | | AA 361- | | 011514581.1 |
| | optimized | protein | | 551 | | AA |
| | ZF6/Notch1NLS/p65- | | | | | 318-370 |
| | MLXIPL | | | | | |
| | SynTF | | | | | |
| pDP1765 | HingeNotch | Reference | N/A | UNIPROT | N/A | NCBI |
| | with 4 RAM | synthetic | | Q04206: | | XP_{—} |
| | residues, | zinc finger | | AA 361- | | 011514581.1 |
| | optimized | protein | | 551 | | AA |
| | ZF6/Notch1NLS/p65- | | | | | 318-370 |
| | MLXIPL | | | | | |
| | SynTF | | | | | |
| pDP1766 | HingeNotch | Reference | N/A | UNIPROT | N/A | NCBI |
| | with 5 RAM | synthetic | | Q04206: | | XP_{—} |
| | residues, | zinc finger | | AA 361- | | 011514581.1 |
| | optimized | protein | | 551 | | AA |
| | ZF6/Notch1NLS/p65- | | | | | 318-370 |
| | MLXIPL | | | | | |
| | SynTF | | | | | |
| pDP1767 | HingeNotch | Reference | N/A | UNIPROT | N/A | NCBI |
| | with 6 RAM | synthetic | | Q04206: | | XP_{—} |
| | residues, | zinc finger | | AA 361- | | 011514581.1 |
| | optimized | protein | | 551 | | AA |
| | ZF6/Notch1NLS/p65- | | | | | 318-370 |
| | MLXIPL | | | | | |
| | SynTF | | | | | |
| pDP1768 | HingeNotch | Reference | N/A | UNIPROT | N/A | NCBI |
| | with 7 RAM | synthetic | | Q04206: | | XP_{—} |
| | residues, | zinc finger | | AA 361- | | 011514581.1 |
| | optimized | protein | | 551 | | AA |
| | ZF6/Notch1NLS/p65- | | | | | 318-370 |
| | MLXIPL | | | | | |
| | SynTF | | | | | |
| pDP1725 | HingeNotch | Reference | N/A | UNIPROT | N/A | NCBI |
| | with 8 RAM | synthetic | | Q04206: | | XP_{—} |
| | residues, | zinc finger | | AA 361- | | 011514581.1 |
| | optimized | protein | | 551 | | AA |
| | ZF6/Notch1NLS/p65- | | | | | 318-370 |
| | MLXIPL | | | | | |
| | SynTF | | | | | |
| pDP1769 | HingeNotch | Reference | N/A | UNIPROT | N/A | NCBI |
| | with 9 RAM | synthetic | | Q04206: | | XP_{—} |
| | residues, | zinc finger | | AA 361- | | 011514581.1 |
| | optimized | protein | | 551 | | AA |
| | ZF6/Notch1NLS/p65- | | | | | 318-370 |
| | MLXIPL | | | | | |
| | SynTF | | | | | |
| pDP1770 | HingeNotch | Reference | N/A | UNIPROT | N/A | NCBI |
| | with 10 | synthetic | | Q04206: | | XP_{—} |
| | RAM | zinc finger | | AA 361- | | 011514581.1 |
| | residues, | protein | | 551 | | AA |
| | optimized | | | | | 318-370 |
| | ZF6/Notch1NLS/p65- | | | | | |
| | MLXIPL | | | | | |
| | SynTF | | | | | |
| pDP1771 | HingeNotch | Reference | N/A | UNIPROT | N/A | NCBI |
| | with 11 | synthetic | | Q04206: | | XP_{—} |
| | RAM | zinc finger | | AA 361- | | 011514581.1 |
| | residues, | protein | | 551 | | AA |
| | optimized | | | | | 318-370 |
| | ZF6/Notch1NLS/p65- | | | | | |
| | MLXIPL | | | | | |
| | SynTF | | | | | |

### Primary Human T Cell Isolation and Culture

Primary CD4+ or CD8+ T cells were isolated from anonymous donor blood after apheresis by negative selection (STEMCELL Technologies #15062 & 15063). Blood was obtained from Blood Centers of the Pacific (San Francisco, CA) as approved by the University Institutional Review Board. T cells were cryopreserved in RPMI-1640 (UCSF cell culture core) with 20% human AB serum (Valley Biomedical Inc., #HP1022) and 10% DMSO. After thawing, T cells were cultured in human T cell medium consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

Lentiviral Transduction of Human T cells

Pantropic VSV-G pseudotyped lentivirus was produced via transfection of Lenti-X 293T cells (Clontech #1113ID) with a transgene expression vector constructed as described above and the viral packaging plasmids pCMVdR8.91 and pMD2.G using Mirus TransIT-Lenti (Mirus #MIR 6606). Primary T cells were thawed the same day, and after 24 hours in culture, were stimulated with Human T-Activator CD3/CD28 Dynabeads (Life Technologies #11131D) at a 1:3 cell:bead ratio. At 48 hours, viral supernatant was harvested and the primary T cells were exposed to the virus for 24 hours. At day 5 post T cell stimulation, the Dynabeads were removed, and the T cells expanded until day 10 when they were rested and could be used in assays. T cells were sorted for assays with a Beckton Dickinson (BD) FACs ARIA II.

### Cancer Cell Lines

The target cell lines for all experiments are K562 myelogenous leukemia cells (ATCC #CCL-243). K562^{CD19+} were generated via lentiviral transduction to stably express human CD19 at equivalent levels as Daudi tumors. CD19 levels were determined by staining the cells with α-CD19 APC (Biolegend #302212). K562^{ALPPL2+}, K562^{BCMA+}, and K562^{CD19+/BCMA+} cells were similarly generated via lentiviral transduction.

### In Vitro Stimulation of Primary T Cells

For all in vitro T cell stimulations, 1×10⁵ T cells were co-cultured with sender cells at a 1:1 ratio in round bottom 96-well tissue culture plates. The cultures were analyzed at 72 hours for reporter activation with a BD Fortessa X-50. All flow cytometry analysis was performed in FlowJo software (TreeStar).

### In Vivo Experiments

8-12 week old NOD.Cg-Prkdc^{scid}Il2rg^{tm1Wjl}/SzJ (NSG) mice were implanted with 1E6 K562^{BCMA} or K562^{BCMA/CD19} tumor cells subcutaneously. Four days after tumor implantation, 6E6 engineered primary human T-cells (unsorted mixed population of CD4⁺ and CD8⁺) were infused retroorbitally. Tumor size was monitored via caliper and calculated as ½×width²×length. Animal experiments were conducted under approval from the UCSF Institutional Animal Care and Use Committee.

### Example 6: In Vivo Efficacy of Humanized ZFTF Hingenotches

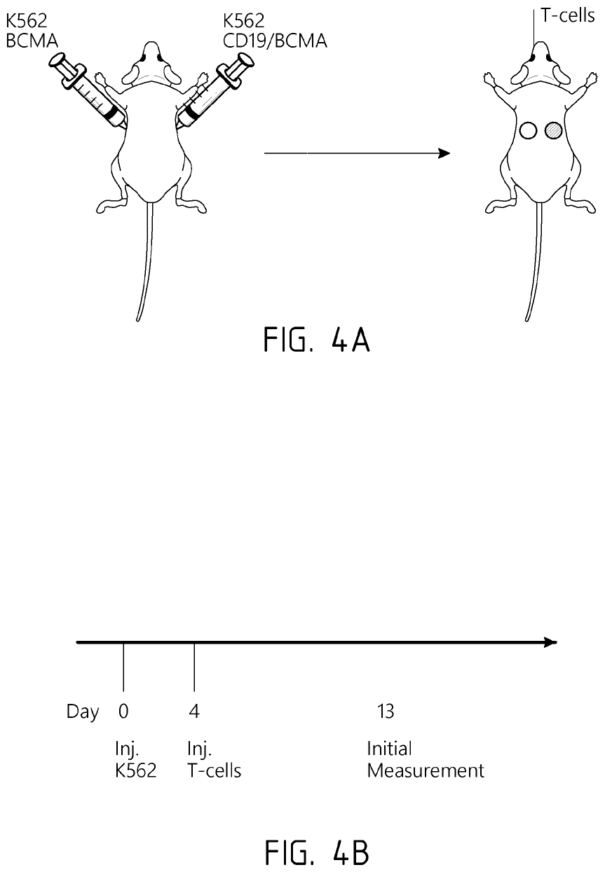

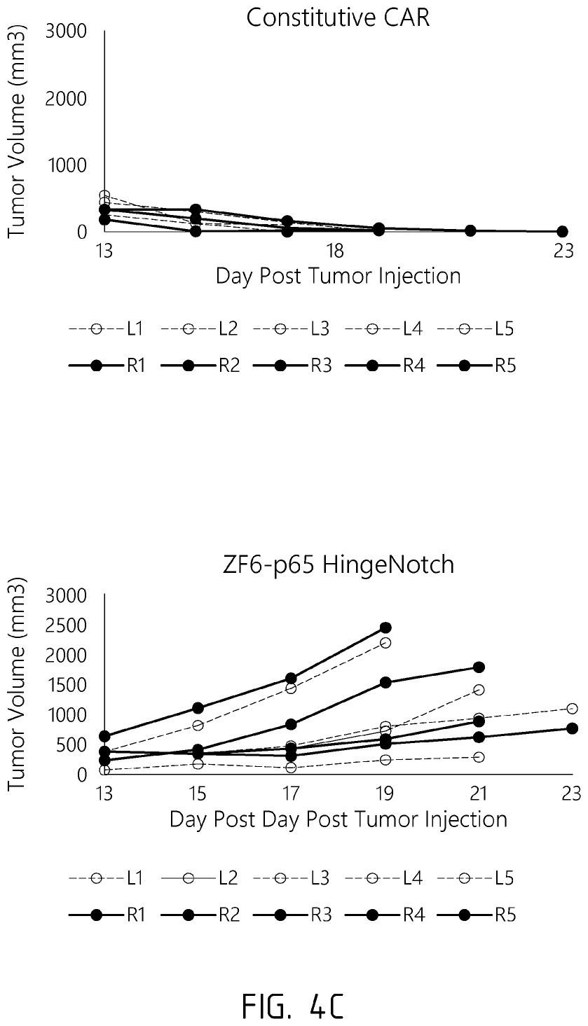

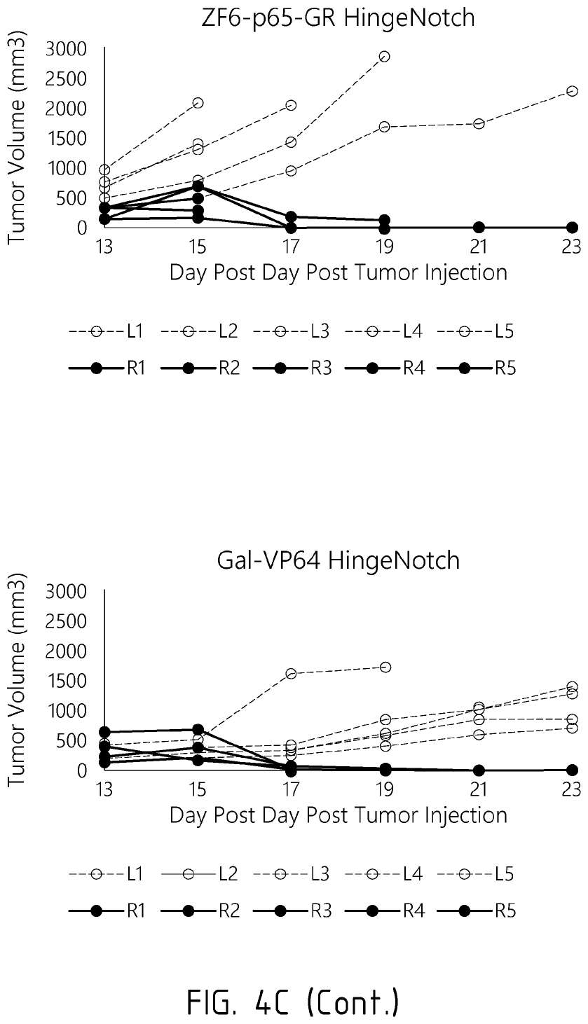

This experiment compares the potency of the original humanized HingeNotch design with the p65 TAD, and the augmented humanized HingeNotch design with the tandem p65-GR TAD. And experimental layout is shown in FIG. 4A, and an experimental timeline is shown in FIG. 4B. The constitutive CAR is a control which should, and does, kill all tumors expressing BCMA (e.g. both tumors). The HingeNotch→CAR circuits are designed to detect a second cancer signal (e.g. CD19) and drive production of the BCMA CAR, thereby only killing the right flank (CD19+BCMA+) tumors. The results show that the original design is insufficiently potent to kill the double-positive tumors, but the new p65-GR TAD enables humanized HingeNotch to control tumor outgrowth comparably to the original Gal-VP64 receptor (FIG. 4C). Importantly, the HingeNotch→CAR T-cells do not kill the tumors that lack the HingeNotch target (grey curves), confirming that the killing of the CD19/BCMA tumors depends on the CD19 HingeNotch priming target.

### Example 7: Additional Transactivation Domain Optimization

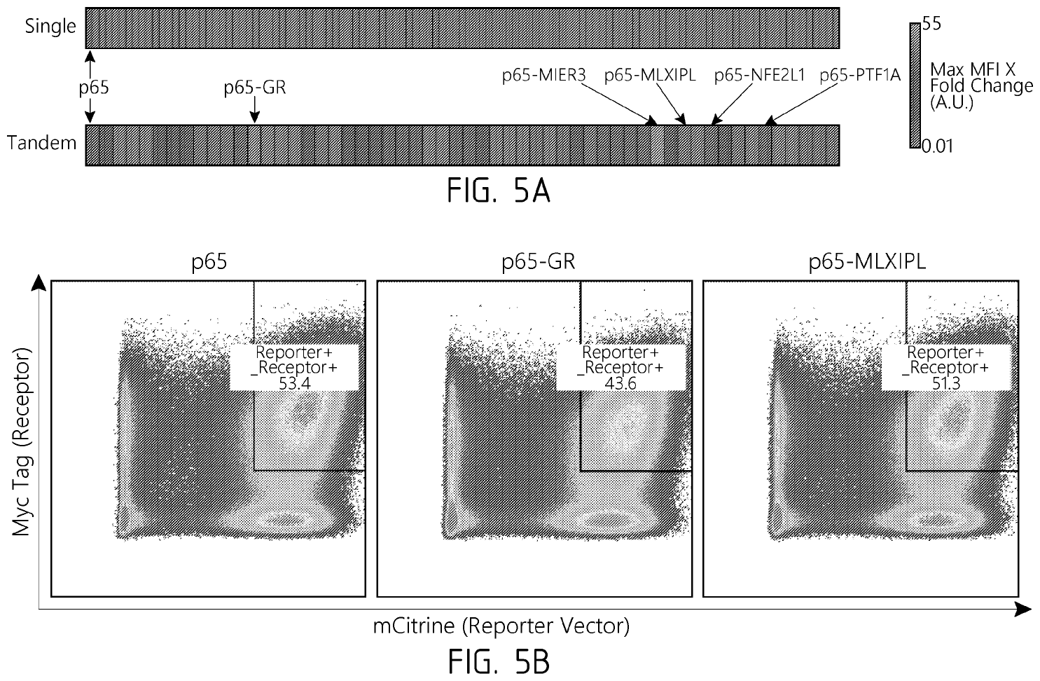

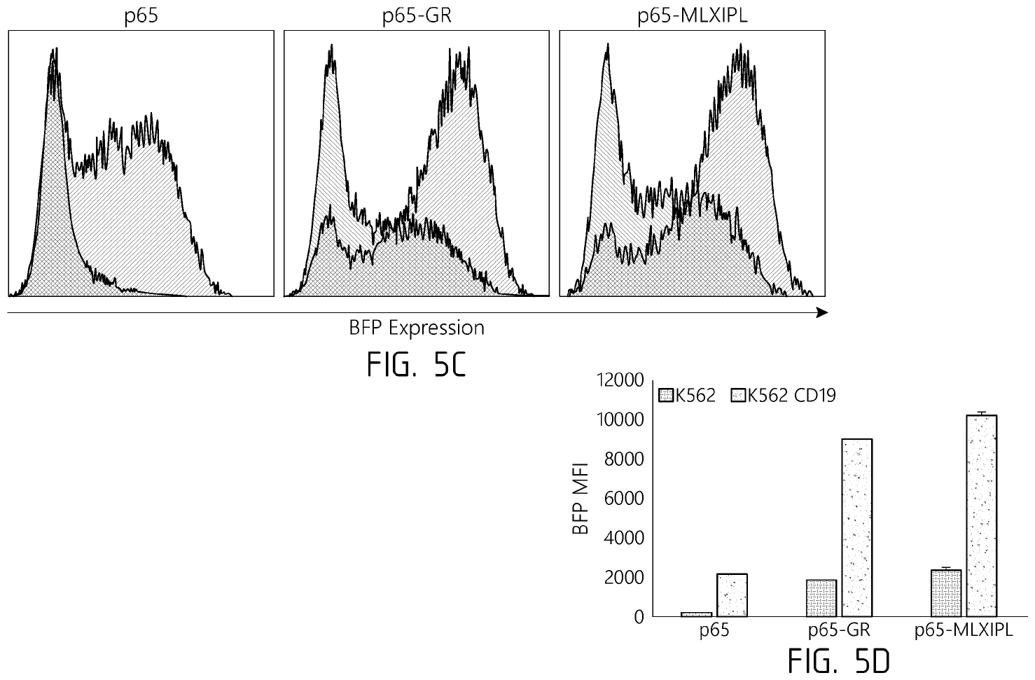

This experiment summarizes a set of experiments designed to find the optimal TAD. FIG. 5A is a summary describing the performance of a large number of receptors, with best performance being red (bright in greyscale) and worst performance being blue (dark in greyscale). Each bar shows the performance of one type of receptor. The top row are receptors where the original p65 TAD is replaced with a different human-derived TAD. The bottom row are receptors where the new human-derived TAD is fused to the end of the p65 TAD. Only a few receptors (all with the fusion design) show desirable activation performance, and are indicated by name. FIG. 5B shows the relative expression of two of the best-performing fusion TADs: p65-GR and p65-MLXIPL. p65-GR improves performance relative to the original p65, but the expression level is lower than p65. In comparison, the p65-MLXIPL fusion has almost identical expression to the original p65. FIGS. 5C-5D show the HingeNotch activation profiles in the presence of target-negative (K562) and target-positive (K562 CD19). Both tandem TADs activate much more strongly than p65 alone, with p65-MLIXPL slightly outperforming p65-GR.

### Example 8: Structural Optimization of ZFTF-Bearing Hingenotches

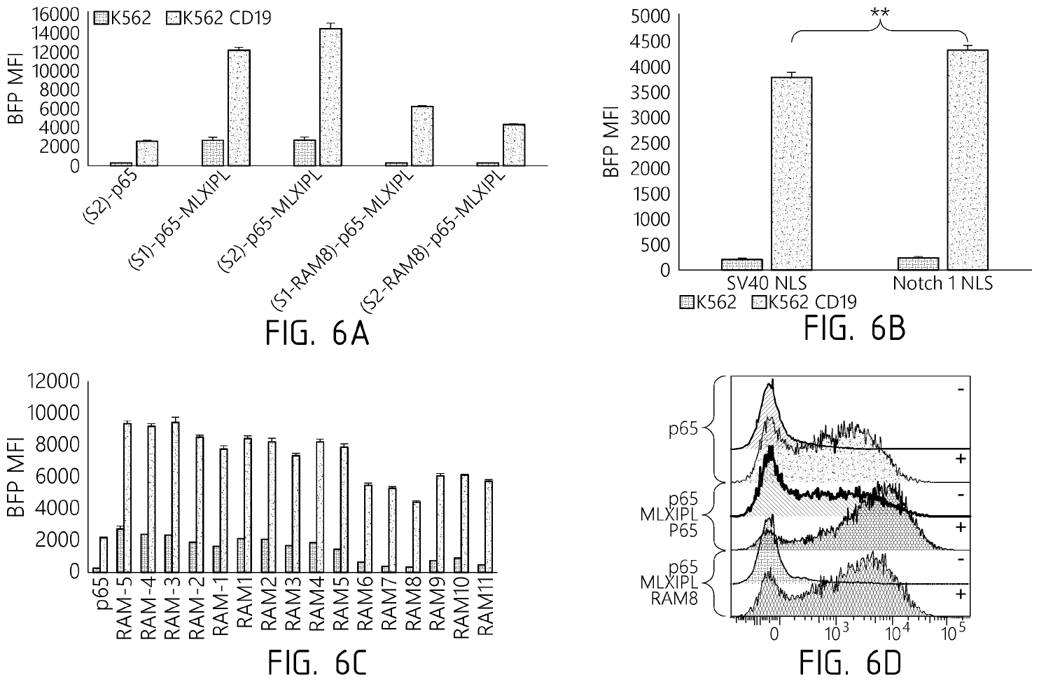

While the results in FIG. 5 show that the tandem TADs are much more active than the original p65, they also can have a stronger baseline signal in the absence of the target antigen, which is often an undesirable property. FIGS. 6A-6D depicts the structural optimization of the humanized HingeNotch receptor to compensate for the additional baseline activation and further improve performance. FIG. 6A compares the original p65 TAD with p65-MLIXPL attached to HingeNotches containing an STS domain from either Notch1 or Notch2, and also with the addition of 8 amino acids from the Notch1 RAM domain. Receptors containing p65-MLXIPL+RAM8 are stronger than the original p65 version, and have less basal signaling than the p65-MLXIPL without RAM. FIG. 6B shows the HingeNotch Receptors bearing: Notch1 STS, RAM8, p65-MLXIPL, while varying the NLS between the original SV40 nuclear localization sequence and the human Notch1 nuclear localization sequence. This panel shows that the Notch1 NLS results in slightly more performant receptors than the original SV40 NLS. Additionally, the Notch1 NLS is human-derived and replaces the final exogenously derived component in the SynTF-containing receptors. FIG. 6C compares the performance of receptors based on the combined properties of those described in FIG. 6A and FIG. 6B. Aside from the original reference receptor (“p65”), these receptors all contain Notch1 NLS and the human p65-MLXIPL TAD. The variants contain segments of the Notch1 RAM region, testing to see which RAM fragment results in optimal activity. The “RAM-5”, “RAM-4” . . . variants not only lack RAM, but also lack the upstream STS region (either the whole region in the case of RAM-5, or fragments of it in the case of RAM-4, -3, etc). Within this panel, RAM7 and RAM8 appear optimal, showing significantly improved activation relative to the p65 reference, and almost no additional basal activity. FIG. 6D shows the activation histograms highlighting the comparison between the original p65 receptor version and the new variants with and without RAM.

### Example 9: Additional P65 Tad Fusions Testing with Pax6-Based Receptors in a Single Vector Format

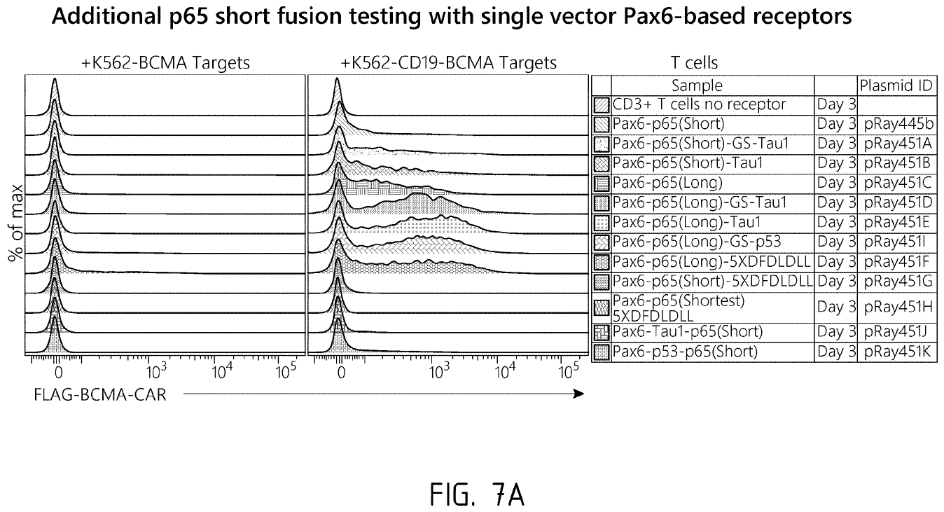

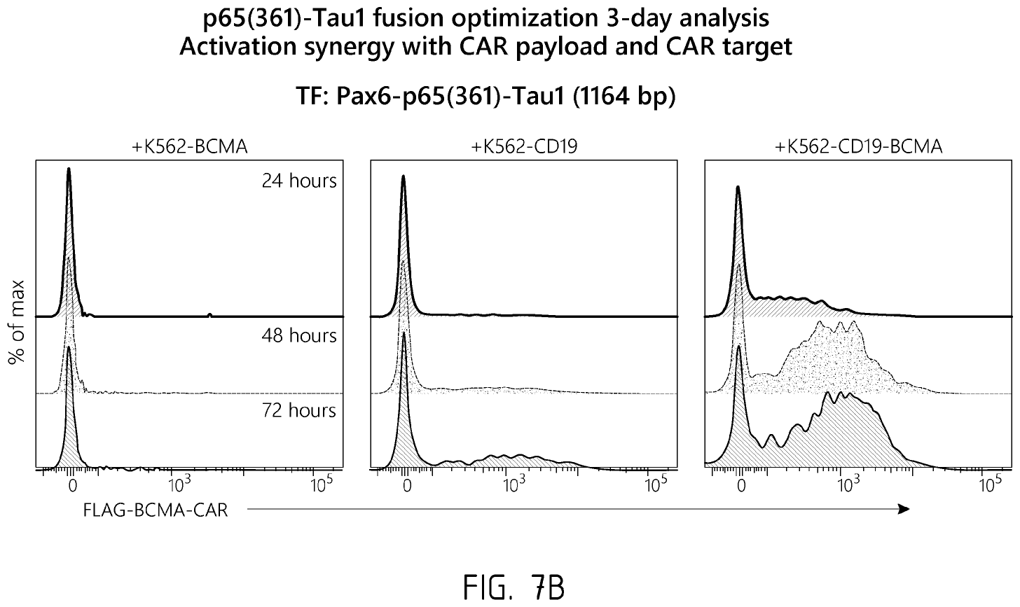

This experiment examines a second set of engineered TADs in the context of the Pax6 transcription factor, and also in the context of a single vector format (FIGS. 7A-7B). Receptors were constructed with the Pax6 TF DNA-binding domain along with a TAD. When activated by the K562-CD19-BCMA target cells, the p65 short and long—only receptors show activity, with p65(long) greater than p65(short). Tandem TADs fused to either p65(Short) and p65(Long) enhances activity, without significantly enhancing background activity as demonstrated by the CD19-negative K562 target cells (left panel) (FIG. 7A).

As shown in FIG. 7B, synergistic effects of the circuit are examined. Since the circuits tested in FIG. 7A produce a BCMA CAR as the output, it was examined whether having the CAR antigen (BCMA) present in the target cells makes a difference in the activation dynamics. The presence of the CAR antigen on the target cells greatly enhances circuit activity.

SEQUENCE TABLE

| | | SEQ ID |
|---|---|---|
| DESCRIPTION | SEQUENCE | NO |
| ECD (for CD19) | MALPVTALLLPLALLLHAARPEQKLISEEDLDIQMTQTTSSLSASLGDRVTISCRAS | 1 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSS | |
| ECD (for APPL2) | MALPVTALLLPLALLLHAARPEQKLISEEDLQVQLQQSGGGLVKPGGSLRLSCAA | 2 |
| | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVL | |
| LINKING | TTTPAPRPPTPAPTIASQPLSLRPEAC | 3 |
| POLYPEPTIDE | | |
| TMD | FMYVAAAAFVLLFFVGCGVLLS | 4 |
| STS | KRKRKH | 5 |
| DBD (ZF3) | APKKKRKVGIHGVPGGLEGGGGSGGTASSRPGERPFQCRICMRNFSQRSSLVRHT | 6 |
| | RTHTGEKPFQCRICMRNFSDKSVLARHLRTHTGSQKPFQCRICMRNFSQRSSLVR | |
| | HTRTHTGEKPFQCRICMRNFSQRNNLGRHLRTHTGSQKPFQCRICMRNFSTHAVL | |
| | TRHTRTHTGEKPFQCRICMRNFSDRGNLTRHLRTHLR | |
| DBD (ZF6) | APKKKRKVGIHGVPGGLEGGGGSGGTASSRPGERPFQCRICMRNFSQQTNLTRHT | 7 |
| | RTHTGEKPFQCRICMRNFSQKANLGVHLRTHTGSQKPFQCRICMRNFSDHSSLKR | |
| | HTRTHTGEKPFQCRICMRNFSRADMLRRHLRTHTGSQKPFQCRICMRNFSEKQNL | |
| | ARHTRTHTGEKPFQCRICMRNFSDPSNLRRHLRTHLR | |
| DBD | PKKKRKVSRPGERPFQCRICMRNFSQQTNLTRHTRTHTGEKPFQCRICMRNFSQK | 8 |
| (ZF6/SV40NLS) | ANLGVHLRTHTGSQKPFQCRICMRNFSDHSSLKRHTRTHTGEKPFQCRICMRNFS | |
| | RADMLRRHLRTHTGSQKPFQCRICMRNFSEKQNLARHTRTHTGEKPFQCRICMR | |
| | NFSDPSNLRRHLRTHLR | |
| DBD | RRKKSRPGERPFQCRICMRNFSQQTNLTRHTRTHTGEKPFQCRICMRNFSQKANL | 9 |
| (ZF6/Notch1NLS) | GVHLRTHTGSQKPFQCRICMRNFSDHSSLKRHTRTHTGEKPFQCRICMRNFSRAD | |
| | MLRRHLRTHTGSQKPFQCRICMRNFSEKQNLARHTRTHTGEKPFQCRICMRNFSD | |
| | PSNLRRHLRTHLR | |
| DBD | PKKKRKVSRPGERPFQCRICMRNFSRRHGLDRHTRTHTGEKPFQCRICMRNFSDH | 10 |
| (ZF10/SV40NLS) | SSLKRHLRTHTGSQKPFQCRICMRNFSVRHNLTRHLRTHTGEKPFQCRICMRNFS | |
| | DHSNLSRHLKTHTGSQKPFQCRICMRNFSQRSSLVRHLRTHTGEKPFQCRICMRN | |
| | FSESGHLKRHLRTHLR | |
| DBD | RRKKSRPGERPFQCRICMRNFSRRHGLDRHTRTHTGEKPFQCRICMRNFSDHSSL | 11 |
| (ZF10/Notch1NLS) | KRHLRTHTGSQKPFQCRICMRNFSVRHNLTRHLRTHTGEKPFQCRICMRNFSDHS | |
| | NLSRHLKTHTGSQKPFQCRICMRNFSQRSSLVRHLRTHTGEKPFQCRICMRNFSES | |
| | GHLKRHLRTHLR | |
| DBD (PAX6) | MQNSHSGVNQLGGVFVNGRPLPDSTRQKIVELAHSGARPCDISRILQVSNGCVSKI | 12 |
| | LGRYYETGSIRPRAIGGSKPRVATPEVVSKIAQYKRECPSIFAWEIRDRLLSEGVCT | |
| | NDNIPSVSSINRVLRNLASEKQQMGA | |
| Linker-1 | GSTCR | 13 |
| Linker-1 variant | M | 14 |
| TAD (mouse p65) | PSGQISNQALALAPSSAPVLAQTMVPSSAMVPLAQPPAPAPVLTPGPPQSLSAPVP | 15 |
| | KSTQAGEGTLSEALLHLQFDADEDLGALLGNSTDPGVFTDLASVDNSEFQQLLNQ | |
| | GVSMSHSTAEPMLMEYPEAITRLVTGSQRPPDPAPTPLGTSGLPNGLSGDEDFSSI | |
| | ADMDFSALLSQISS | |
| TAD [human p65 | PSGQISQASALAPAPPQVLPQAPAPAPAPAMVSALAQAPAPVPVLAPGPPQAVAPP | 16 |
| (mouse-homologous | APKPTQAGEGTLSEALLQLQFDDEDLGALLGNSTDPAVFTDLASVDNSEFQQLLN | |
| region)] | QGIPVAPHTTEPMLMEYPEAITRLVTGAQRPPDPAPAPLGAPGLPNGLLSGDEDFS | |
| | SIADMDFSALLSQISS | |
| TAD (human p65) | DEFPTMVFPSGQISQASALAPAPPQVLPQAPAPAPAPAMVSALAQAPAPVPVLAP | 17 |
| | GPPQAVAPPAPKPTQAGEGTLSEALLQLQFDDEDLGALLGNSTDPAVFTDLASVD | |
| | NSEFQQLLNQGIPVAPHTTEPMLMEYPEAITRLVTGAQRPPDPAPAPLGAPGLPNG | |
| | LLSGDEDFSSIADMDFSALLSQISS | |
| TAD (human p65, | PTQAGEGTLSEALLQLQFDDEDLGALLGNSTDPAVFTDLASVDNSEFQQLLNQGI | 18 |
| minimized TAD1 + | PVAPHTTEPMLMEYPEAITRLVTGAQRPPDPAPAPLGAPGLPNGLLSGDEDFSSIA | |
| TAD2) | DMDFSALLSQISS | |
| TAD (human p65, | PGLPNGLLSGDEDFSSIADMDFSALLSQISS | 19 |
| minimized TAD1) | | |
| TAD (human GR | DQSTFDILQDLEFSSGSPGKETNESPWRSDLLIDENCLLSPLAGEDDSFLLEGNSNE | 20 |
| Tau1, minimal) | D | |
| TAD (human p53, | DDLMLSPDDIEQWFTEDPGPDEAPRMPEAAPPVAPAPAAPTPAAP | 21 |
| TAD2) | | |
| TAD [human ATF6 | DFDLDLLPDFDLDLLPDFDLDLLPDFDLDLLP | 22 |
| 8aa fragment (4x | | |
| repeats)] | | |
| Linker −2 | SGQGGGGS | 23 |
| Additional | GFSVDTSALLDLFSPSVTVPDMSLPDLDSSLASIQELLSPQEPPRPPEAENSSPDSGK | 24 |
| Transcriptional | QLVHYTAQPLFLLDPGSVDTGSNDLPVLFELGEGSYFSEGDGFAEDPTISLLTGSEP | |
| Effector (human | PKAKDPTVS | |
| HSF1 TAD) | | |
| Additional | GLSHSYMDAAQYPLPEEVDVLFNIDGQGNHVPPYYGNSVRATVQR | 25 |
| Transcriptional | | |
| Effector (human | | |
| GATA3, TAD1) | | |
| Additional | EEPQSDPSVEPPLSQETFSDLWKLLPENNVLSPLPSQAM | 26 |
| Transcriptional | | |
| Effector (human | | |
| p53, TAD1) | | |
| Additional | DDLMLSPDDIEQWFTEDPGPDEAPRMPEAAPPVAPAPAAPTPAAP | 27 |
| Transcriptional | | |
| Effector (human | | |
| p53, TAD2) | | |
| Additional | SMDESGLPQLTSYDCEVNAPIQGSRNLLQGEELLRALDQVN | 28 |
| Transcriptional | | |
| Effector (human | | |
| HIF1a, CAD) | | |
| Additional | DQSTFDILQDLEFSSGSPGKETNESPWRSDLLIDENCLLSPLAGEDDSFLLEGNSNE | 29 |
| Transcriptional | D | |
| Effector (human | | |
| GR Taul (minimal), | | |
| TAD) | | |
| Additional | DFDLDLLDFDLDLLDFDLDLLDFDLDLLDFDLDLL | 30 |
| Transcriptional | | |
| Effector (human | | |
| ATF6 7aa, TAD, 5x | | |
| repeats) | | |
| Additional | TSSSSDELSWIIELLEKDGMAFQEALDPGPFTSSSSDELSWIIELLEKDGMAFQEAL | 31 |
| Transcriptional | DPGPF | |
| Effector (human | | |
| ELF3, TAD, 2x | | |
| repeats) | | |
| Additional | DDLMLSPDDIEQWFTEDPGPDEAPRMPEAAPPVAPAPAAPTPAAP | 32 |
| Transcriptional | | |
| Effector (human | | |
| p53, TAD2) | | |
| Additional | PTQAGEGTLSEALLQLQFDDEDLGALLGNSTDPAVFTDLASVDNSEFQQLLNQGI | 33 |
| Transcriptional | PVAPHTTEPMLMEYPEAITRLVTGAQRPPDPAPAPLGAPGLPNGLLSGDEDFSSIA | |
| Effector [human | DMDFSALLSQISS | |
| p65 (428)] | | |
| Additional | DEFPTMVFPSGQISQASALAPAPPQVLPQAPAPAPAPAMVSALAQAPAPVPVLAP | 34 |
| Transcriptional | GPPQAVAPPAPKPTQAGEGTLSEALLQLQFDDEDLGALLGNSTDPAVFTDLASVD | |
| Effector [human | NSEFQQLLNQGIPVAPHTTEPMLMEYPEAITRLVTGAQRPPDPAPAPLGAPGLPNG | |
| p65 (361)] | LLSGDEDFSSIADMDFSALLSQISS | |
| FULL SEQUENCE | MALPVTALLLPLALLLHAARPEQKLISEEDLDIQMTQTTSSLSASLGDRVTISCRAS | 35 |
| (pDP1179) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQRSSLVRHTRTHT | |
| | GEKPFQCRICMRNFSDKSVLARHLRTHTGSQKPFQCRICMRNFSQRSSLVRHTRT | |
| | HTGEKPFQCRICMRNFSQRNNLGRHLRTHTGSQKPFQCRICMRNFSTHAVLTRHT | |
| | RTHTGEKPFQCRICMRNFSDRGNLTRHLRTHLRGSTCRPSGQISNQALALAPSSAP | |
| | VLAQTMVPSSAMVPLAQPPAPAPVLTPGPPQSLSAPVPKSTQAGEGTLSEALLHL | |
| | QFDADEDLGALLGNSTDPGVFTDLASVDNSEFQQLLNQGVSMSHSTAEPMLMEY | |
| | PEAITRLVTGSQRPPDPAPTPLGTSGLPNGLSGDEDFSSIADMDFSALLSQISS | |
| FULL SEQUENCE | MALPVTALLLPLALLLHAARPEQKLISEEDLDIQMTQTTSSLSASLGDRVTISCRAS | 36 |
| (pDP1180) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQRSSLVRHTRTHT | |
| | GEKPFQCRICMRNFSDKSVLARHLRTHTGSQKPFQCRICMRNFSQRSSLVRHTRT | |
| | HTGEKPFQCRICMRNFSQRNNLGRHLRTHTGSQKPFQCRICMRNFSTHAVLTRHT | |
| | RTHTGEKPFQCRICMRNFSDRGNLTRHLRTHLRGSTCRPSGQISQASALAPAPPQV | |
| | LPQAPAPAPAPAMVSALAQAPAPVPVLAPGPPQAVAPPAPKPTQAGEGTLSEALL | |
| | QLQFDDEDLGALLGNSTDPAVFTDLASVDNSEFQQLLNQGIPVAPHTTEPMLMEY | |
| | PEAITRLVTGAQRPPDPAPAPLGAPGLPNGLLSGDEDFSSIADMDFSALLSQISS | |
| FULL SEQUENCE | MALPVTALLLPLALLLHAARPEQKLISEEDLDIQMTQTTSSLSASLGDRVTISCRAS | 37 |
| (pDP1181) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQRSSLVRHTRTHT | |
| | GEKPFQCRICMRNFSDKSVLARHLRTHTGSQKPFQCRICMRNFSQRSSLVRHTRT | |
| | HTGEKPFQCRICMRNFSQRNNLGRHLRTHTGSQKPFQCRICMRNFSTHAVLTRHT | |
| | RTHTGEKPFQCRICMRNFSDRGNLTRHLRTHLRGSTCRDEFPTMVFPSGQISQASA | |
| | LAPAPPQVLPQAPAPAPAPAMVSALAQAPAPVPVLAPGPPQAVAPPAPKPTQAGE | |
| | GTLSEALLQLQFDDEDLGALLGNSTDPAVFTDLASVDNSEFQQLLNQGIPVAPHT | |
| | TEPMLMEYPEAITRLVTGAQRPPDPAPAPLGAPGLPNGLLSGDEDFSSIADMDFSA | |
| | LLSQISSSGQGGGGSGFSVDTSALLDLFSPSVTVPDMSLPDLDSSLASIQELLSPQEP | |
| | PRPPEAENSSPDSGKQLVHYTAQPLFLLDPGSVDTGSNDLPVLFELGEGSYFSEGD | |
| | GFAEDPTISLLTGSEPPKAKDPTVS | |
| FULL SEQUENCE | MALPVTALLLPLALLLHAARPEQKLISEEDLDIQMTQTTSSLSASLGDRVTISCRAS | 38 |
| (pDP1182) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQRSSLVRHTRTHT | |
| | GEKPFQCRICMRNFSDKSVLARHLRTHTGSQKPFQCRICMRNFSQRSSLVRHTRT | |
| | HTGEKPFQCRICMRNFSQRNNLGRHLRTHTGSQKPFQCRICMRNFSTHAVLTRHT | |
| | RTHTGEKPFQCRICMRNFSDRGNLTRHLRTHLRGSTCRPSGQISQASALAPAPPQV | |
| | LPQAPAPAPAPAMVSALAQAPAPVPVLAPGPPQAVAPPAPKPTQAGEGTLSEALL | |
| | QLQFDDEDLGALLGNSTDPAVFTDLASVDNSEFQQLLNQGIPVAPHTTEPMLMEY | |
| | PEAITRLVTGAQRPPDPAPAPLGAPGLPNGLLSGDEDFSSIADMDFSALLSQISSSG | |
| | QGGGGSGFSVDTSALLDLFSPSVTVPDMSLPDLDSSLASIQELLSPQEPPRPPEAEN | |
| | SSPDSGKQLVHYTAQPLFLLDPGSVDTGSNDLPVLFELGEGSYFSEGDGFAEDPTI | |
| | SLLTGSEPPKAKDPTVS | |
| FULL SEQUENCE | MALPVTALLLPLALLLHAARPEQKLISEEDLDIQMTQTTSSLSASLGDRVTISCRAS | 39 |
| (pDP1160) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQQTNLTRHTRTHT | |
| | GEKPFQCRICMRNFSQKANLGVHLRTHTGSQKPFQCRICMRNFSDHSSLKRHTRT | |
| | HTGEKPFQCRICMRNFSRADMLRRHLRTHTGSQKPFQCRICMRNFSEKQNLARHT | |
| | RTHTGEKPFQCRICMRNFSDPSNLRRHLRTHLRGSTCRDEFPTMVFPSGQISQASA | |
| | LAPAPPQVLPQAPAPAPAPAMVSALAQAPAPVPVLAPGPPQAVAPPAPKPTQAGE | |
| | GTLSEALLQLQFDDEDLGALLGNSTDPAVFTDLASVDNSEFQQLLNQGIPVAPHT | |
| | TEPMLMEYPEAITRLVTGAQRPPDPAPAPLGAPGLPNGLLSGDEDFSSIADMDFSA | |
| | LLSQISS | |
| FULL SEQUENCE | MALPVTALLLPLALLLHAARPEQKLISEEDLDIQMTQTTSSLSASLGDRVTISCRAS | 40 |
| (pDP1174) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQQTNLTRHTRTHT | |
| | GEKPFQCRICMRNFSQKANLGVHLRTHTGSQKPFQCRICMRNFSDHSSLKRHTRT | |
| | HTGEKPFQCRICMRNFSRADMLRRHLRTHTGSQKPFQCRICMRNFSEKQNLARHT | |
| | RTHTGEKPFQCRICMRNFSDPSNLRRHLRTHLRGSTCRPSGQISNQALALAPSSAP | |
| | VLAQTMVPSSAMVPLAQPPAPAPVLTPGPPQSLSAPVPKSTQAGEGTLSEALLHL | |
| | QFDADEDLGALLGNSTDPGVFTDLASVDNSEFQQLLNQGVSMSHSTAEPMLMEY | |
| | PEAITRLVTGSQRPPDPAPTPLGTSGLPNGLSGDEDFSSIADMDFSALLSQISSSGQG | |
| | GGGSGFSVDTSALLDLFSPSVTVPDMSLPDLDSSLASIQELLSPQEPPRPPEAENSSP | |
| | DSGKQLVHYTAQPLFLLDPGSVDTGSNDLPVLFELGEGSYFSEGDGFAEDPTISLL | |
| | TGSEPPKAKDPTVS | |
| FULL SEQUENCE | MALPVTALLLPLALLLHAARPEQKLISEEDLDIQMTQTTSSLSASLGDRVTISCRAS | 41 |
| (pDP1279) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQQTNLTRHTRTHT | |
| | GEKPFQCRICMRNFSQKANLGVHLRTHTGSQKPFQCRICMRNFSDHSSLKRHTRT | |
| | HTGEKPFQCRICMRNFSRADMLRRHLRTHTGSQKPFQCRICMRNFSEKQNLARHT | |
| | RTHTGEKPFQCRICMRNFSDPSNLRRHLRTHLRGSTCRDEFPTMVFPSGQISQASA | |
| | LAPAPPQVLPQAPAPAPAPAMVSALAQAPAPVPVLAPGPPQAVAPPAPKPTQAGE | |
| | GTLSEALLQLQFDDEDLGALLGNSTDPAVFTDLASVDNSEFQQLLNQGIPVAPHT | |
| | TEPMLMEYPEAITRLVTGAQRPPDPAPAPLGAPGLPNGLLSGDEDFSSIADMDFSA | |
| | LLSQISSSGQGGGGSGLSHSYMDAAQYPLPEEVDVLFNIDGQGNHVPPYYGNSVR | |
| | ATVQR | |
| FULL SEQUENCE | MALPVTALLLPLALLLHAARPEQKLISEEDLDIQMTQTTSSLSASLGDRVTISCRAS | 42 |
| (pDP1280) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQQTNLTRHTRTHT | |
| | GEKPFQCRICMRNFSQKANLGVHLRTHTGSQKPFQCRICMRNFSDHSSLKRHTRT | |
| | HTGEKPFQCRICMRNFSRADMLRRHLRTHTGSQKPFQCRICMRNFSEKQNLARHT | |
| | RTHTGEKPFQCRICMRNFSDPSNLRRHLRTHLRGSTCRDEFPTMVFPSGQISQASA | |
| | LAPAPPQVLPQAPAPAPAPAMVSALAQAPAPVPVLAPGPPQAVAPPAPKPTQAGE | |
| | GTLSEALLQLQFDDEDLGALLGNSTDPAVFTDLASVDNSEFQQLLNQGIPVAPHT | |
| | TEPMLMEYPEAITRLVTGAQRPPDPAPAPLGAPGLPNGLLSGDEDFSSIADMDFSA | |
| | LLSQISSSGQGGGGSEEPQSDPSVEPPLSQETFSDLWKLLPENNVLSPLPSQAM | |
| FULL SEQUENCE | MALPVTALLLPLALLLHAARPEQKLISEEDLDIQMTQTTSSLSASLGDRVTISCRAS | 43 |
| (pDP1281) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQQTNLTRHTRTHT | |
| | GEKPFQCRICMRNFSQKANLGVHLRTHTGSQKPFQCRICMRNFSDHSSLKRHTRT | |
| | HTGEKPFQCRICMRNFSRADMLRRHLRTHTGSQKPFQCRICMRNFSEKQNLARHT | |
| | RTHTGEKPFQCRICMRNFSDPSNLRRHLRTHLRGSTCRDEFPTMVFPSGQISQASA | |
| | LAPAPPQVLPQAPAPAPAPAMVSALAQAPAPVPVLAPGPPQAVAPPAPKPTQAGE | |
| | GTLSEALLQLQFDDEDLGALLGNSTDPAVFTDLASVDNSEFQQLLNQGIPVAPHT | |
| | TEPMLMEYPEAITRLVTGAQRPPDPAPAPLGAPGLPNGLLSGDEDFSSIADMDFSA | |
| | LLSQISSSGQGGGGSDDLMLSPDDIEQWFTEDPGPDEAPRMPEAAPPVAPAPAAPT | |
| | PAAP | |
| FULL SEQUENCE | MALPVTALLLPLALLLHAARPEQKLISEEDLDIQMTQTTSSLSASLGDRVTISCRAS | 44 |
| (pDP1285) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQQTNLTRHTRTHT | |
| | GEKPFQCRICMRNFSQKANLGVHLRTHTGSQKPFQCRICMRNFSDHSSLKRHTRT | |
| | HTGEKPFQCRICMRNFSRADMLRRHLRTHTGSQKPFQCRICMRNFSEKQNLARHT | |
| | RTHTGEKPFQCRICMRNFSDPSNLRRHLRTHLRGSTCRDEFPTMVFPSGQISQASA | |
| | LAPAPPQVLPQAPAPAPAPAMVSALAQAPAPVPVLAPGPPQAVAPPAPKPTQAGE | |
| | GTLSEALLQLQFDDEDLGALLGNSTDPAVFTDLASVDNSEFQQLLNQGIPVAPHT | |
| | TEPMLMEYPEAITRLVTGAQRPPDPAPAPLGAPGLPNGLLSGDEDFSSIADMDFSA | |
| | LLSQISSSGQGGGGSSMDESGLPQLTSYDCEVNAPIQGSRNLLQGEELLRALDQVN | |
| FULL SEQUENCE | MALPVTALLLPLALLLHAARPEQKLISEEDLDIQMTQTTSSLSASLGDRVTISCRAS | 45 |
| (pDP1286) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQQTNLTRHTRTHT | |
| | GEKPFQCRICMRNFSQKANLGVHLRTHTGSQKPFQCRICMRNFSDHSSLKRHTRT | |
| | HTGEKPFQCRICMRNFSRADMLRRHLRTHTGSQKPFQCRICMRNFSEKQNLARHT | |
| | RTHTGEKPFQCRICMRNFSDPSNLRRHLRTHLRGSTCRDEFPTMVFPSGQISQASA | |
| | LAPAPPQVLPQAPAPAPAPAMVSALAQAPAPVPVLAPGPPQAVAPPAPKPTQAGE | |
| | GTLSEALLQLQFDDEDLGALLGNSTDPAVFTDLASVDNSEFQQLLNQGIPVAPHT | |
| | TEPMLMEYPEAITRLVTGAQRPPDPAPAPLGAPGLPNGLLSGDEDFSSIADMDFSA | |
| | LLSQISSSGQGGGGSDQSTFDILQDLEFSSGSPGKETNESPWRSDLLIDENCLLSPL | |
| | AGEDDSFLLEGNSNED | |
| FULL SEQUENCE | MALPVTALLLPLALLLHAARPEQKLISEEDLDIQMTQTTSSLSASLGDRVTISCRAS | 46 |
| (pDP1290) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQQTNLTRHTRTHT | |
| | GEKPFQCRICMRNFSQKANLGVHLRTHTGSQKPFQCRICMRNFSDHSSLKRHTRT | |
| | HTGEKPFQCRICMRNFSRADMLRRHLRTHTGSQKPFQCRICMRNFSEKQNLARHT | |
| | RTHTGEKPFQCRICMRNFSDPSNLRRHLRTHLRGSTCRDEFPTMVFPSGQISQASA | |
| | LAPAPPQVLPQAPAPAPAPAMVSALAQAPAPVPVLAPGPPQAVAPPAPKPTQAGE | |
| | GTLSEALLQLQFDDEDLGALLGNSTDPAVFTDLASVDNSEFQQLLNQGIPVAPHT | |
| | TEPMLMEYPEAITRLVTGAQRPPDPAPAPLGAPGLPNGLLSGDEDFSSIADMDFSA | |
| | LLSQISSSGQGGGGSDFDLDLLDFDLDLLDFDLDLLDFDLDLLDFDLDLL | |
| FULL SEQUENCE | MALPVTALLLPLALLLHAARPEQKLISEEDLDIQMTQTTSSLSASLGDRVTISCRAS | 47 |
| (pDP1292) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHAPKK | |
| | KRKVGIHGVPGGLEGGGGSGGTASSRPGERPFQCRICMRNFSQQTNLTRHTRTHT | |
| | GEKPFQCRICMRNFSQKANLGVHLRTHTGSQKPFQCRICMRNFSDHSSLKRHTRT | |
| | HTGEKPFQCRICMRNFSRADMLRRHLRTHTGSQKPFQCRICMRNFSEKQNLARHT | |
| | RTHTGEKPFQCRICMRNFSDPSNLRRHLRTHLRGSTCRDEFPTMVFPSGQISQASA | |
| | LAPAPPQVLPQAPAPAPAPAMVSALAQAPAPVPVLAPGPPQAVAPPAPKPTQAGE | |
| | GTLSEALLQLQFDDEDLGALLGNSTDPAVFTDLASVDNSEFQQLLNQGIPVAPHT | |
| | TEPMLMEYPEAITRLVTGAQRPPDPAPAPLGAPGLPNGLLSGDEDFSSIADMDFSA | |
| | LLSQISSSGQGGGGSTSSSSDELSWIIELLEKDGMAFQEALDPGPFTSSSSDELSWII | |
| | ELLEKDGMAFQEALDPGPF | |
| FULL SEQUENCE | MALPVTALLLPLALLLHAARPEQKLISEEDLQVQLQQSGGGLVKPGGSLRLSCAA | 48 |
| (pDP1338) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHPKKKRKVSRPGERPFQCRICMRNFSQQTNLTRHTRTHTGEKPFQCRICMRNF | |
| | SQKANLGVHLRTHTGSQKPFQCRICMRNFSDHSSLKRHTRTHTGEKPFQCRICMR | |
| | NFSRADMLRRHLRTHTGSQKPFQCRICMRNFSEKQNLARHTRTHTGEKPFQCRIC | |
| | MRNFSDPSNLRRHLRTHLRDEFPTMVFPSGQISQASALAPAPPQVLPQAPAPAPAP | |
| | AMVSALAQAPAPVPVLAPGPPQAVAPPAPKPTQAGEGTLSEALLQLQFDDEDLG | |
| | ALLGNSTDPAVFTDLASVDNSEFQQLLNQGIPVAPHTTEPMLMEYPEAITRLVTG | |
| | AQRPPDPAPAPLGAPGLPNGLLSGDEDFSSIADMDFSALLSQISS | |
| FULL SEQUENCE | MALPVTALLLPLALLLHAARPEQKLISEEDLQVQLQQSGGGLVKPGGSLRLSCAA | 49 |
| (pDP1339) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHPKKKRKVSRPGERPFQCRICMRNFSQQTNLTRHTRTHTGEKPFQCRICMRNF | |
| | SQKANLGVHLRTHTGSQKPFQCRICMRNFSDHSSLKRHTRTHTGEKPFQCRICMR | |
| | NFSRADMLRRHLRTHTGSQKPFQCRICMRNFSEKQNLARHTRTHTGEKPFQCRIC | |
| | MRNFSDPSNLRRHLRTHLRDEFPTMVFPSGQISQASALAPAPPQVLPQAPAPAPAP | |
| | AMVSALAQAPAPVPVLAPGPPQAVAPPAPKPTQAGEGTLSEALLQLQFDDEDLG | |
| | ALLGNSTDPAVFTDLASVDNSEFQQLLNQGIPVAPHTTEPMLMEYPEAITRLVTG | |
| | AQRPPDPAPAPLGAPGLPNGLLSGDEDFSSIADMDFSALLSQISSSGQGGGGSGFS | |
| | VDTSALLDLFSPSVTVPDMSLPDLDSSLASIQELLSPQEPPRPPEAENSSPDSGKQL | |
| | VHYTAQPLFLLDPGSVDTGSNDLPVLFELGEGSYFSEGDGFAEDPTISLLTGSEPPK | |
| | AKDPTVS | |
| FULL SEQUENCE | MALPVTALLLPLALLLHAARPEQKLISEEDLQVQLQQSGGGLVKPGGSLRLSCAA | 50 |
| (pDP1340) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHPKKKRKVSRPGERPFQCRICMRNFSQQTNLTRHTRTHTGEKPFQCRICMRNF | |
| | SQKANLGVHLRTHTGSQKPFQCRICMRNFSDHSSLKRHTRTHTGEKPFQCRICMR | |
| | NFSRADMLRRHLRTHTGSQKPFQCRICMRNFSEKQNLARHTRTHTGEKPFQCRIC | |
| | MRNFSDPSNLRRHLRTHLRDEFPTMVFPSGQISQASALAPAPPQVLPQAPAPAPAP | |
| | AMVSALAQAPAPVPVLAPGPPQAVAPPAPKPTQAGEGTLSEALLQLQFDDEDLG | |
| | ALLGNSTDPAVFTDLASVDNSEFQQLLNQGIPVAPHTTEPMLMEYPEAITRLVTG | |
| | AQRPPDPAPAPLGAPGLPNGLLSGDEDFSSIADMDFSALLSQISSSGQGGGGSDQS | |
| | TFDILQDLEFSSGSPGKETNESPWRSDLLIDENCLLSPLAGEDDSFLLEGNSNED | |
| FULL SEQUENCE | MALPVTALLLPLALLLHAARPEQKLISEEDLQVQLQQSGGGLVKPGGSLRLSCAA | 51 |
| (pDP1341) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHRRKKSRPGERPFQCRICMRNFSQQTNLTRHTRTHTGEKPFQCRICMRNFSQK | |
| | ANLGVHLRTHTGSQKPFQCRICMRNFSDHSSLKRHTRTHTGEKPFQCRICMRNFS | |
| | RADMLRRHLRTHTGSQKPFQCRICMRNFSEKQNLARHTRTHTGEKPFQCRICMR | |
| | NFSDPSNLRRHLRTHLRDEFPTMVFPSGQISQASALAPAPPQVLPQAPAPAPAPAM | |
| | VSALAQAPAPVPVLAPGPPQAVAPPAPKPTQAGEGTLSEALLQLQFDDEDLGALL | |
| | GNSTDPAVFTDLASVDNSEFQQLLNQGIPVAPHTTEPMLMEYPEAITRLVTGAQR | |
| | PPDPAPAPLGAPGLPNGLLSGDEDFSSIADMDFSALLSQISS | |
| FULL SEQUENCE | MALPVTALLLPLALLLHAARPEQKLISEEDLQVQLQQSGGGLVKPGGSLRLSCAA | 52 |
| (pDP1342) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHRRKKSRPGERPFQCRICMRNFSQQTNLTRHTRTHTGEKPFQCRICMRNFSQK | |
| | ANLGVHLRTHTGSQKPFQCRICMRNFSDHSSLKRHTRTHTGEKPFQCRICMRNFS | |
| | RADMLRRHLRTHTGSQKPFQCRICMRNFSEKQNLARHTRTHTGEKPFQCRICMR | |
| | NFSDPSNLRRHLRTHLRDEFPTMVFPSGQISQASALAPAPPQVLPQAPAPAPAPAM | |
| | VSALAQAPAPVPVLAPGPPQAVAPPAPKPTQAGEGTLSEALLQLQFDDEDLGALL | |
| | GNSTDPAVFTDLASVDNSEFQQLLNQGIPVAPHTTEPMLMEYPEAITRLVTGAQR | |
| | PPDPAPAPLGAPGLPNGLLSGDEDFSSIADMDFSALLSQISSSGQGGGGSGFSVDTS | |
| | ALLDLFSPSVTVPDMSLPDLDSSLASIQELLSPQEPPRPPEAENSSPDSGKQLVHYT | |
| | AQPLFLLDPGSVDTGSNDLPVLFELGEGSYFSEGDGFAEDPTISLLTGSEPPKAKDP | |
| | TVS | |
| FULL SEQUENCE | MALPVTALLLPLALLLHAARPEQKLISEEDLQVQLQQSGGGLVKPGGSLRLSCAA | 53 |
| (pDP1343) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHRRKKSRPGERPFQCRICMRNFSQQTNLTRHTRTHTGEKPFQCRICMRNFSQK | |
| | ANLGVHLRTHTGSQKPFQCRICMRNFSDHSSLKRHTRTHTGEKPFQCRICMRNFS | |
| | RADMLRRHLRTHTGSQKPFQCRICMRNFSEKQNLARHTRTHTGEKPFQCRICMR | |
| | NFSDPSNLRRHLRTHLRDEFPTMVFPSGQISQASALAPAPPQVLPQAPAPAPAPAM | |
| | VSALAQAPAPVPVLAPGPPQAVAPPAPKPTQAGEGTLSEALLQLQFDDEDLGALL | |
| | GNSTDPAVFTDLASVDNSEFQQLLNQGIPVAPHTTEPMLMEYPEAITRLVTGAQR | |
| | PPDPAPAPLGAPGLPNGLLSGDEDFSSIADMDFSALLSQISSSGQGGGGSDQSTFDI | |
| | LQDLEFSSGSPGKETNESPWRSDLLIDENCLLSPLAGEDDSFLLEGNSNED | |
| FULL SEQUENCE | MALPVTALLLPLALLLHAARPEQKLISEEDLQVQLQQSGGGLVKPGGSLRLSCAA | 54 |
| (pDP1344) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHPKKKRKVSRPGERPFQCRICMRNFSRRHGLDRHTRTHTGEKPFQCRICMRNF | |
| | SDHSSLKRHLRTHTGSQKPFQCRICMRNFSVRHNLTRHLRTHTGEKPFQCRICMR | |
| | NFSDHSNLSRHLKTHTGSQKPFQCRICMRNFSQRSSLVRHLRTHTGEKPFQCRICM | |
| | RNFSESGHLKRHLRTHLRDEFPTMVFPSGQISQASALAPAPPQVLPQAPAPAPAPA | |
| | MVSALAQAPAPVPVLAPGPPQAVAPPAPKPTQAGEGTLSEALLQLQFDDEDLGAL | |
| | LGNSTDPAVFTDLASVDNSEFQQLLNQGIPVAPHTTEPMLMEYPEAITRLVTGAQ | |
| | RPPDPAPAPLGAPGLPNGLLSGDEDFSSIADMDFSALLSQISS | |
| FULL SEQUENCE | MALPVTALLLPLALLLHAARPEQKLISEEDLQVQLQQSGGGLVKPGGSLRLSCAA | 55 |
| (pDP1345) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHPKKKRKVSRPGERPFQCRICMRNFSRRHGLDRHTRTHTGEKPFQCRICMRNF | |
| | SDHSSLKRHLRTHTGSQKPFQCRICMRNFSVRHNLTRHLRTHTGEKPFQCRICMR | |
| | NFSDHSNLSRHLKTHTGSQKPFQCRICMRNFSQRSSLVRHLRTHTGEKPFQCRICM | |
| | RNFSESGHLKRHLRTHLRDEFPTMVFPSGQISQASALAPAPPQVLPQAPAPAPAPA | |
| | MVSALAQAPAPVPVLAPGPPQAVAPPAPKPTQAGEGTLSEALLQLQFDDEDLGAL | |
| | LGNSTDPAVFTDLASVDNSEFQQLLNQGIPVAPHTTEPMLMEYPEAITRLVTGAQ | |
| | RPPDPAPAPLGAPGLPNGLLSGDEDFSSIADMDFSALLSQISSSGQGGGGSGFSVDT | |
| | SALLDLFSPSVTVPDMSLPDLDSSLASIQELLSPQEPPRPPEAENSSPDSGKQLVHY | |
| | TAQPLFLLDPGSVDTGSNDLPVLFELGEGSYFSEGDGFAEDPTISLLTGSEPPKAKD | |
| | PTVS | |
| FULL SEQUENCE | MALPVTALLLPLALLLHAARPEQKLISEEDLQVQLQQSGGGLVKPGGSLRLSCAA | 56 |
| (pDP1346) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHPKKKRKVSRPGERPFQCRICMRNFSRRHGLDRHTRTHTGEKPFQCRICMRNF | |
| | SDHSSLKRHLRTHTGSQKPFQCRICMRNFSVRHNLTRHLRTHTGEKPFQCRICMR | |
| | NFSDHSNLSRHLKTHTGSQKPFQCRICMRNFSQRSSLVRHLRTHTGEKPFQCRICM | |
| | RNFSESGHLKRHLRTHLRDEFPTMVFPSGQISQASALAPAPPQVLPQAPAPAPAPA | |
| | MVSALAQAPAPVPVLAPGPPQAVAPPAPKPTQAGEGTLSEALLQLQFDDEDLGAL | |
| | LGNSTDPAVFTDLASVDNSEFQQLLNQGIPVAPHTTEPMLMEYPEAITRLVTGAQ | |
| | RPPDPAPAPLGAPGLPNGLLSGDEDFSSIADMDFSALLSQISSSGQGGGGSDQSTFD | |
| | ILQDLEFSSGSPGKETNESPWRSDLLIDENCLLSPLAGEDDSFLLEGNSNED | |
| FULL SEQUENCE | MALPVTALLLPLALLLHAARPEQKLISEEDLQVQLQQSGGGLVKPGGSLRLSCAA | 57 |
| (pDP1347) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHRRKKSRPGERPFQCRICMRNFSRRHGLDRHTRTHTGEKPFQCRICMRNFSDH | |
| | SSLKRHLRTHTGSQKPFQCRICMRNFSVRHNLTRHLRTHTGEKPFQCRICMRNFS | |
| | DHSNLSRHLKTHTGSQKPFQCRICMRNFSQRSSLVRHLRTHTGEKPFQCRICMRN | |
| | FSESGHLKRHLRTHLRDEFPTMVFPSGQISQASALAPAPPQVLPQAPAPAPAPAMV | |
| | SALAQAPAPVPVLAPGPPQAVAPPAPKPTQAGEGTLSEALLQLQFDDEDLGALLG | |
| | NSTDPAVFTDLASVDNSEFQQLLNQGIPVAPHTTEPMLMEYPEAITRLVTGAQRPP | |
| | DPAPAPLGAPGLPNGLLSGDEDFSSIADMDFSALLSQISS | |
| FULL SEQUENCE | MALPVTALLLPLALLLHAARPEQKLISEEDLQVQLQQSGGGLVKPGGSLRLSCAA | 58 |
| (pDP1348) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHRRKKSRPGERPFQCRICMRNFSRRHGLDRHTRTHTGEKPFQCRICMRNFSDH | |
| | SSLKRHLRTHTGSQKPFQCRICMRNFSVRHNLTRHLRTHTGEKPFQCRICMRNFS | |
| | DHSNLSRHLKTHTGSQKPFQCRICMRNFSQRSSLVRHLRTHTGEKPFQCRICMRN | |
| | FSESGHLKRHLRTHLRDEFPTMVFPSGQISQASALAPAPPQVLPQAPAPAPAPAMV | |
| | SALAQAPAPVPVLAPGPPQAVAPPAPKPTQAGEGTLSEALLQLQFDDEDLGALLG | |
| | NSTDPAVFTDLASVDNSEFQQLLNQGIPVAPHTTEPMLMEYPEAITRLVTGAQRPP | |
| | DPAPAPLGAPGLPNGLLSGDEDFSSIADMDFSALLSQISSSGQGGGGSGFSVDTSA | |
| | LLDLFSPSVTVPDMSLPDLDSSLASIQELLSPQEPPRPPEAENSSPDSGKQLVHYTA | |
| | QPLFLLDPGSVDTGSNDLPVLFELGEGSYFSEGDGFAEDPTISLLTGSEPPKAKDPT | |
| | VS | |
| FULL SEQUENCE | MALPVTALLLPLALLLHAARPEQKLISEEDLQVQLQQSGGGLVKPGGSLRLSCAA | 59 |
| (pDP1349) | SGFTFSSYAMHWVRQAPGKGLEWVAVISYDGSNKYYADSVKGRFTISRDNSKNT | |
| | LYLQMDSLRAEDTAVYYCAKEGDSSRWSYDLWGRGTLVTVSSGGGGSGGGGSG | |
| | GGGSQSALTQPASVSGSPGQSITISCTGTSSDVGGYNYVSWYQQHPGKAPKVMIY | |
| | DVTNRPSGVSNRFSGSKSGNTASLTISGLQAEDEADYYCSSYTIASTLVVFGGGTK | |
| | LTVLTTTPAPRPPTPAPTIASQPLSLRPEACFMYVAAAAFVLLFFVGCGVLLSKRK | |
| | RKHRRKKSRPGERPFQCRICMRNFSRRHGLDRHTRTHTGEKPFQCRICMRNFSDH | |
| | SSLKRHLRTHTGSQKPFQCRICMRNFSVRHNLTRHLRTHTGEKPFQCRICMRNFS | |
| | DHSNLSRHLKTHTGSQKPFQCRICMRNFSQRSSLVRHLRTHTGEKPFQCRICMRN | |
| | FSESGHLKRHLRTHLRDEFPTMVFPSGQISQASALAPAPPQVLPQAPAPAPAPAMV | |
| | SALAQAPAPVPVLAPGPPQAVAPPAPKPTQAGEGTLSEALLQLQFDDEDLGALLG | |
| | NSTDPAVFTDLASVDNSEFQQLLNQGIPVAPHTTEPMLMEYPEAITRLVTGAQRPP | |
| | DPAPAPLGAPGLPNGLLSGDEDFSSIADMDFSALLSQISSSGQGGGGSDQSTFDILQ | |
| | DLEFSSGSPGKETNESPWRSDLLIDENCLLSPLAGEDDSFLLEGNSNED | |
| FULL SEQUENCE | MALPVTALLLPLALLLHAARPEQKLISEEDLDIQMTQTTSSLSASLGDRVTISCRAS | 60 |
| (pRay451A) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMQNS | |
| | HSGVNQLGGVFVNGRPLPDSTRQKIVELAHSGARPCDISRILQVSNGCVSKILGRY | |
| | YETGSIRPRAIGGSKPRVATPEVVSKIAQYKRECPSIFAWEIRDRLLSEGVCTNDNI | |
| | PSVSSINRVLRNLASEKQQMGAPTQAGEGTLSEALLQLQFDDEDLGALLGNSTDP | |
| | AVFTDLASVDNSEFQQLLNQGIPVAPHTTEPMLMEYPEAITRLVTGAQRPPDPAP | |
| | APLGAPGLPNGLLSGDEDFSSIADMDFSALLSQISSSGQGGGGSDQSTFDILQDLEF | |
| | SSGSPGKETNESPWRSDLLIDENCLLSPLAGEDDSFLLEGNSNED | |
| FULL SEQUENCE | MALPVTALLLPLALLLHAARPEQKLISEEDLDIQMTQTTSSLSASLGDRVTISCRAS | 61 |
| (pRay451B) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMQNS | |
| | HSGVNQLGGVFVNGRPLPDSTRQKIVELAHSGARPCDISRILQVSNGCVSKILGRY | |
| | YETGSIRPRAIGGSKPRVATPEVVSKIAQYKRECPSIFAWEIRDRLLSEGVCTNDNI | |
| | PSVSSINRVLRNLASEKQQMGAPTQAGEGTLSEALLQLQFDDEDLGALLGNSTDP | |
| | AVFTDLASVDNSEFQQLLNQGIPVAPHTTEPMLMEYPEAITRLVTGAQRPPDPAP | |
| | APLGAPGLPNGLLSGDEDFSSIADMDFSALLSQISSDQSTFDILQDLEFSSGSPGKET | |
| | NESPWRSDLLIDENCLLSPLAGEDDSFLLEGNSNED | |
| FULL SEQUENCE | MALPVTALLLPLALLLHAARPEQKLISEEDLDIQMTQTTSSLSASLGDRVTISCRAS | 62 |
| (pRay451D) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMQNS | |
| | HSGVNQLGGVFVNGRPLPDSTRQKIVELAHSGARPCDISRILQVSNGCVSKILGRY | |
| | YETGSIRPRAIGGSKPRVATPEVVSKIAQYKRECPSIFAWEIRDRLLSEGVCTNDNI | |
| | PSVSSINRVLRNLASEKQQMGADEFPTMVFPSGQISQASALAPAPPQVLPQAPAPA | |
| | PAPAMVSALAQAPAPVPVLAPGPPQAVAPPAPKPTQAGEGTLSEALLQLQFDDED | |
| | LGALLGNSTDPAVFTDLASVDNSEFQQLLNQGIPVAPHTTEPMLMEYPEAITRLVT | |
| | GAQRPPDPAPAPLGAPGLPNGLLSGDEDFSSIADMDFSALLSQISSSGQGGGGSDQ | |
| | STFDILQDLEFSSGSPGKETNESPWRSDLLIDENCLLSPLAGEDDSFLLEGNSNED | |
| FULL SEQUENCE | MALPVTALLLPLALLLHAARPEQKLISEEDLDIQMTQTTSSLSASLGDRVTISCRAS | 63 |
| (pRay451E) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMQNS | |
| | HSGVNQLGGVFVNGRPLPDSTRQKIVELAHSGARPCDISRILQVSNGCVSKILGRY | |
| | YETGSIRPRAIGGSKPRVATPEVVSKIAQYKRECPSIFAWEIRDRLLSEGVCTNDNI | |
| | PSVSSINRVLRNLASEKQQMGADEFPTMVFPSGQISQASALAPAPPQVLPQAPAPA | |
| | PAPAMVSALAQAPAPVPVLAPGPPQAVAPPAPKPTQAGEGTLSEALLQLQFDDED | |
| | LGALLGNSTDPAVFTDLASVDNSEFQQLLNQGIPVAPHTTEPMLMEYPEAITRLVT | |
| | GAQRPPDPAPAPLGAPGLPNGLLSGDEDFSSIADMDFSALLSQISSDQSTFDILQDL | |
| | EFSSGSPGKETNESPWRSDLLIDENCLLSPLAGEDDSFLLEGNSNED | |
| FULL SEQUENCE | MALPVTALLLPLALLLHAARPEQKLISEEDLDIQMTQTTSSLSASLGDRVTISCRAS | 64 |
| (pRay451F) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMQNS | |
| | HSGVNQLGGVFVNGRPLPDSTRQKIVELAHSGARPCDISRILQVSNGCVSKILGRY | |
| | YETGSIRPRAIGGSKPRVATPEVVSKIAQYKRECPSIFAWEIRDRLLSEGVCTNDNI | |
| | PSVSSINRVLRNLASEKQQMGADEFPTMVFPSGQISQASALAPAPPQVLPQAPAPA | |
| | PAPAMVSALAQAPAPVPVLAPGPPQAVAPPAPKPTQAGEGTLSEALLQLQFDDED | |
| | LGALLGNSTDPAVFTDLASVDNSEFQQLLNQGIPVAPHTTEPMLMEYPEAITRLVT | |
| | GAQRPPDPAPAPLGAPGLPNGLLSGDEDFSSIADMDFSALLSQISSDFDLDLLDFDL | |
| | DLLDFDLDLLDFDLDLLDFDLDLL | |
| FULL SEQUENCE | MALPVTALLLPLALLLHAARPEQKLISEEDLDIQMTQTTSSLSASLGDRVTISCRAS | 65 |
| (pRay451G) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMQNS | |
| | HSGVNQLGGVFVNGRPLPDSTRQKIVELAHSGARPCDISRILQVSNGCVSKILGRY | |
| | YETGSIRPRAIGGSKPRVATPEVVSKIAQYKRECPSIFAWEIRDRLLSEGVCTNDNI | |
| | PSVSSINRVLRNLASEKQQMGAPTQAGEGTLSEALLQLQFDDEDLGALLGNSTDP | |
| | AVFTDLASVDNSEFQQLLNQGIPVAPHTTEPMLMEYPEAITRLVTGAQRPPDPAP | |
| | APLGAPGLPNGLLSGDEDFSSIADMDFSALLSQISSDFDLDLLDFDLDLLDFDLDLL | |
| | DFDLDLLDFDLDLL | |
| FULL SEQUENCE | MALPVTALLLPLALLLHAARPEQKLISEEDLDIQMTQTTSSLSASLGDRVTISCRAS | 66 |
| (pRay451H) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMQNS | |
| | HSGVNQLGGVFVNGRPLPDSTRQKIVELAHSGARPCDISRILQVSNGCVSKILGRY | |
| | YETGSIRPRAIGGSKPRVATPEVVSKIAQYKRECPSIFAWEIRDRLLSEGVCTNDNI | |
| | PSVSSINRVLRNLASEKQQMGAPGLPNGLLSGDEDFSSIADMDFSALLSQISSDFDL | |
| | DLLDFDLDLLDFDLDLLDFDLDLLDFDLDLL | |
| FULL SEQUENCE | MALPVTALLLPLALLLHAARPEQKLISEEDLDIQMTQTTSSLSASLGDRVTISCRAS | 67 |
| (pRay451I) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMQNS | |
| | HSGVNQLGGVFVNGRPLPDSTRQKIVELAHSGARPCDISRILQVSNGCVSKILGRY | |
| | YETGSIRPRAIGGSKPRVATPEVVSKIAQYKRECPSIFAWEIRDRLLSEGVCTNDNI | |
| | PSVSSINRVLRNLASEKQQMGADEFPTMVFPSGQISQASALAPAPPQVLPQAPAPA | |
| | PAPAMVSALAQAPAPVPVLAPGPPQAVAPPAPKPTQAGEGTLSEALLQLQFDDED | |
| | LGALLGNSTDPAVFTDLASVDNSEFQQLLNQGIPVAPHTTEPMLMEYPEAITRLVT | |
| | GAQRPPDPAPAPLGAPGLPNGLLSGDEDFSSIADMDFSALLSQISSSGQGGGGSDD | |
| | LMLSPDDIEQWFTEDPGPDEAPRMPEAAPPVAPAPAAPTPAAP | |
| FULL SEQUENCE | MALPVTALLLPLALLLHAARPEQKLISEEDLDIQMTQTTSSLSASLGDRVTISCRAS | 68 |
| (pRay451J) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHDQSTF | |
| | DILQDLEFSSGSPGKETNESPWRSDLLIDENCLLSPLAGEDDSFLLEGNSNEDMQN | |
| | SHSGVNQLGGVFVNGRPLPDSTRQKIVELAHSGARPCDISRILQVSNGCVSKILGR | |
| | YYETGSIRPRAIGGSKPRVATPEVVSKIAQYKRECPSIFAWEIRDRLLSEGVCTNDN | |
| | IPSVSSINRVLRNLASEKQQMGAPTQAGEGTLSEALLQLQFDDEDLGALLGNSTDP | |
| | AVFTDLASVDNSEFQQLLNQGIPVAPHTTEPMLMEYPEAITRLVTGAQRPPDPAP | |
| | APLGAPGLPNGLLSGDEDFSSIADMDFSALLSQISS | |
| FULL SEQUENCE | MALPVTALLLPLALLLHAARPEQKLISEEDLDIQMTQTTSSLSASLGDRVTISCRAS | 69 |
| (pRay451K) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMDDL | |
| | MLSPDDIEQWFTEDPGPDEAPRMPEAAPPVAPAPAAPTPAAPMQNSHSGVNQLG | |
| | GVFVNGRPLPDSTRQKIVELAHSGARPCDISRILQVSNGCVSKILGRYYETGSIRPR | |
| | AIGGSKPRVATPEVVSKIAQYKRECPSIFAWEIRDRLLSEGVCTNDNIPSVSSINRV | |
| | LRNLASEKQQMGAPTQAGEGTLSEALLQLQFDDEDLGALLGNSTDPAVFTDLAS | |
| | VDNSEFQQLLNQGIPVAPHTTEPMLMEYPEAITRLVTGAQRPPDPAPAPLGAPGLP | |
| | NGLLSGDEDFSSIADMDFSALLSQISS | |
| FULL SEQUENCE | MALPVTALLLPLALLLHAARPEQKLISEEDLDIQMTQTTSSLSASLGDRVTISCRAS | 70 |
| (pRay451L) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMQNS | |
| | HSGVNQLGGVFVNGRPLPDSTRQKIVELAHSGARPCDISRILQVSNGCVSKILGRY | |
| | YETGSIRPRAIGGSKPRVATPEVVSKIAQYKRECPSIFAWEIRDRLLSEGVCTNDNI | |
| | PSVSSINRVLRNLASEKQQMGADFDLDLLPDFDLDLLPDFDLDLLPDFDLDLLPPT | |
| | QAGEGTLSEALLQLQFDDEDLGALLGNSTDPAVFTDLASVDNSEFQQLLNQGIPV | |
| | APHTTEPMLMEYPEAITRLVTGAQRPPDPAPAPLGAPGLPNGLLSGDEDFSSIADM | |
| | DFSALLSQISS | |
| FULL SEQUENCE | MALPVTALLLPLALLLHAARPEQKLISEEDLDIQMTQTTSSLSASLGDRVTISCRAS | 71 |
| (pRay451M) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHMQNS | |
| | HSGVNQLGGVFVNGRPLPDSTRQKIVELAHSGARPCDISRILQVSNGCVSKILGRY | |
| | YETGSIRPRAIGGSKPRVATPEVVSKIAQYKRECPSIFAWEIRDRLLSEGVCTNDNI | |
| | PSVSSINRVLRNLASEKQQMGADFDLDLLPDFDLDLLPDFDLDLLPDFDLDLLPDE | |
| | FPTMVFPSGQISQASALAPAPPQVLPQAPAPAPAPAMVSALAQAPAPVPVLAPGPP | |
| | QAVAPPAPKPTQAGEGTLSEALLQLQFDDEDLGALLGNSTDPAVFTDLASVDNSE | |
| | FQQLLNQGIPVAPHTTEPMLMEYPEAITRLVTGAQRPPDPAPAPLGAPGLPNGLLS | |
| | GDEDFSSIADMDFSALLSQISS | |
| Linker variant | GSGSGSGS | 72 |
| CD8a signal | MALPVTALLLPLALLLHAARP | 73 |
| peptide | | |
| Myc-tag | EQKLISEEDL | 74 |
| STS variant 1 | RKRRR | 75 |
| STS variant 2 | R | 76 |
| STS variant 3 | RK | 77 |
| STS variant 4 | RKR | 78 |
| STS variant 5 | RKRR | 79 |
| RAM | QHGQLWFP | 80 |
| RAM variant 1 | Q | 81 |
| RAM variant 2 | QH | 82 |
| RAM variant 3 | QHG | 83 |
| RAM variant 4 | QHGQ | 84 |
| RAM variant 5 | QHGQL | 85 |
| RAM variant 6 | QHGQLW | 86 |
| RAM variant 7 | QHGQLWF | 87 |
| RAM variant 8 | QHGQLWFPE | 88 |
| RAM variant 9 | QHGQLWFPEG | 89 |
| RAM variant 10 | QHGQLWFPEGF | 90 |
| NLS | PKKKRKV | 91 |
| NLS 1 | RRKK | 92 |
| DBD | SRPGERPFQCRICMRNFSQQTNLTRHTRTHTGEKPFQCRICMRNFSQKANLGVHL | 93 |
| (ZF6/SV40NLS) | RTHTGSQKPFQCRICMRNFSDHSSLKRHTRTHTGEKPFQCRICMRNFSRADMLRR | |
| | HLRTHTGSQKPFQCRICMRNFSEKQNLARHTRTHTGEKPFQCRICMRNFSDPSNL | |
| | RRHLRTHLR | |
| TAD (human GR | DQSTFDILQDLEFSSGSPGKETNESPWRSDLLIDENCLLSPLAGEDDSFLLEGNSNE | 94 |
| TAD1) | D | |
| TAD (human | DYDDERTLEEEEMMDEGKNFSSEIEDLEKEGTMPLEDLLAFYGYEPTIPAVAN | 95 |
| MIER3 TAD | | |
| fragmnet) | | |
| TAD (human | SDTLFTMTQSGPSPLQLPPEDAYVGNADMIQPDLTPLQPSLDDFMDISDFFTN | 96 |
| MLXIPL TAD | | |
| fragment) | | |
| TAD (human | RESETEQGFGEDLEDLGAVAPPVSGDLTKEDIDLIDILWRQDIDLGAGREVFD | 97 |
| NFE2L1 TAD | | |
| fragment) | | |
| TAD human PTF1A | DAVLLEHFPGGLDAFPSSYFDEDDFFTDQSSRDPLEDGDELLADEQAEVEFLS | 98 |
| TAD fragmnet | | |
| FULL SEQUENCE | MALPVTALLLPLALLLHAARPEQKLISEEDLDIQMTQTTSSLSASLGDRVTISCRAS | 99 |
| (pDP1441) | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHPKKK | |
| | RKVSRPGERPFQCRICMRNFSQQTNLTRHTRTHTGEKPFQCRICMRNFSQKANLG | |
| | VHLRTHTGSQKPFQCRICMRNFSDHSSLKRHTRTHTGEKPFQCRICMRNFSRADM | |
| | LRRHLRTHTGSQKPFQCRICMRNFSEKQNLARHTRTHTGEKPFQCRICMRNFSDP | |
| | SNLRRHLRTHLRDEFPTMVFPSGQISQASALAPAPPQVLPQAPAPAPAPAMVSAL | |
| | AQAPAPVPVLAPGPPQAVAPPAPKPTQAGEGTLSEALLQLQFDDEDLGALLGNST | |
| | DPAVFTDLASVDNSEFQQLLNQGIPVAPHTTEPMLMEYPEAITRLVTGAQRPPDP | |
| | APAPLGAPGLPNGLLSGDEDFSSIADMDFSALLSQISS | |
| pDP1487 | MALPVTALLLPLALLLHAARPEQKLISEEDLDIQMTQTTSSLSASLGDRVTISCRAS | 100 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHPKKK | |
| | RKVSRPGERPFQCRICMRNFSQQTNLTRHTRTHTGEKPFQCRICMRNFSQKANLG | |
| | VHLRTHTGSQKPFQCRICMRNFSDHSSLKRHTRTHTGEKPFQCRICMRNFSRADM | |
| | LRRHLRTHTGSQKPFQCRICMRNFSEKQNLARHTRTHTGEKPFQCRICMRNFSDP | |
| | SNLRRHLRTHLRDEFPTMVFPSGQISQASALAPAPPQVLPQAPAPAPAPAMVSAL | |
| | AQAPAPVPVLAPGPPQAVAPPAPKPTQAGEGTLSEALLQLQFDDEDLGALLGNST | |
| | DPAVFTDLASVDNSEFQQLLNQGIPVAPHTTEPMLMEYPEAITRLVTGAQRPPDP | |
| | APAPLGAPGLPNGLLSGDEDFSSIADMDFSALLSQISSDQSTFDILQDLEFSSGSPG | |
| | KETNESPWRSDLLIDENCLLSPLAGEDDSFLLEGNSNED | |
| pDP1558 | MALPVTALLLPLALLLHAARPEQKLISEEDLDIQMTQTTSSLSASLGDRVTISCRAS | 101 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHPKKK | |
| | RKVSRPGERPFQCRICMRNFSQQTNLTRHTRTHTGEKPFQCRICMRNFSQKANLG | |
| | VHLRTHTGSQKPFQCRICMRNFSDHSSLKRHTRTHTGEKPFQCRICMRNFSRADM | |
| | LRRHLRTHTGSQKPFQCRICMRNFSEKQNLARHTRTHTGEKPFQCRICMRNFSDP | |
| | SNLRRHLRTHLRDEFPTMVFPSGQISQASALAPAPPQVLPQAPAPAPAPAMVSAL | |
| | AQAPAPVPVLAPGPPQAVAPPAPKPTQAGEGTLSEALLQLQFDDEDLGALLGNST | |
| | DPAVFTDLASVDNSEFQQLLNQGIPVAPHTTEPMLMEYPEAITRLVTGAQRPPDP | |
| | APAPLGAPGLPNGLLSGDEDFSSIADMDFSALLSQISSDYDDERTLEEEEMMDEGK | |
| | NFSSEIEDLEKEGTMPLEDLLAFYGYEPTIPAVAN | |
| pDP1560 | MALPVTALLLPLALLLHAARPEQKLISEEDLDIQMTQTTSSLSASLGDRVTISCRAS | 102 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHPKKK | |
| | RKVSRPGERPFQCRICMRNFSQQTNLTRHTRTHTGEKPFQCRICMRNFSQKANLG | |
| | VHLRTHTGSQKPFQCRICMRNFSDHSSLKRHTRTHTGEKPFQCRICMRNFSRADM | |
| | LRRHLRTHTGSQKPFQCRICMRNFSEKQNLARHTRTHTGEKPFQCRICMRNFSDP | |
| | SNLRRHLRTHLRDEFPTMVFPSGQISQASALAPAPPQVLPQAPAPAPAPAMVSAL | |
| | AQAPAPVPVLAPGPPQAVAPPAPKPTQAGEGTLSEALLQLQFDDEDLGALLGNST | |
| | DPAVFTDLASVDNSEFQQLLNQGIPVAPHTTEPMLMEYPEAITRLVTGAQRPPDP | |
| | APAPLGAPGLPNGLLSGDEDFSSIADMDFSALLSQISSSDTLFTMTQSGPSPLQLPP | |
| | EDAYVGNADMIQPDLTPLQPSLDDFMDISDFFTN | |
| pDP1562 | MALPVTALLLPLALLLHAARPEQKLISEEDLDIQMTQTTSSLSASLGDRVTISCRAS | 103 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHPKKK | |
| | RKVSRPGERPFQCRICMRNFSQQTNLTRHTRTHTGEKPFQCRICMRNFSQKANLG | |
| | VHLRTHTGSQKPFQCRICMRNFSDHSSLKRHTRTHTGEKPFQCRICMRNFSRADM | |
| | LRRHLRTHTGSQKPFQCRICMRNFSEKQNLARHTRTHTGEKPFQCRICMRNFSDP | |
| | SNLRRHLRTHLRDEFPTMVFPSGQISQASALAPAPPQVLPQAPAPAPAPAMVSAL | |
| | AQAPAPVPVLAPGPPQAVAPPAPKPTQAGEGTLSEALLQLQFDDEDLGALLGNST | |
| | DPAVFTDLASVDNSEFQQLLNQGIPVAPHTTEPMLMEYPEAITRLVTGAQRPPDP | |
| | APAPLGAPGLPNGLLSGDEDFSSIADMDFSALLSQISSRESETEQGFGEDLEDLGA | |
| | VAPPVSGDLTKEDIDLIDILWRQDIDLGAGREVFD | |
| pDP1566 | MALPVTALLLPLALLLHAARPEQKLISEEDLDIQMTQTTSSLSASLGDRVTISCRAS | 104 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHPKKK | |
| | RKVSRPGERPFQCRICMRNFSQQTNLTRHTRTHTGEKPFQCRICMRNFSQKANLG | |
| | VHLRTHTGSQKPFQCRICMRNFSDHSSLKRHTRTHTGEKPFQCRICMRNFSRADM | |
| | LRRHLRTHTGSQKPFQCRICMRNFSEKQNLARHTRTHTGEKPFQCRICMRNFSDP | |
| | SNLRRHLRTHLRDEFPTMVFPSGQISQASALAPAPPQVLPQAPAPAPAPAMVSAL | |
| | AQAPAPVPVLAPGPPQAVAPPAPKPTQAGEGTLSEALLQLQFDDEDLGALLGNST | |
| | DPAVFTDLASVDNSEFQQLLNQGIPVAPHTTEPMLMEYPEAITRLVTGAQRPPDP | |
| | APAPLGAPGLPNGLLSGDEDFSSIADMDFSALLSQISSDAVLLEHFPGGLDAFPSSY | |
| | FDEDDFFTDQSSRDPLEDGDELLADEQAEVEFLS | |
| pDP1673 | MALPVTALLLPLALLLHAARPEQKLISEEDLDIQMTQTTSSLSASLGDRVTISCRAS | 105 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRPKKKRK | |
| | VSRPGERPFQCRICMRNFSQQTNLTRHTRTHTGEKPFQCRICMRNFSQKANLGVH | |
| | LRTHTGSQKPFQCRICMRNFSDHSSLKRHTRTHTGEKPFQCRICMRNFSRADMLR | |
| | RHLRTHTGSQKPFQCRICMRNFSEKQNLARHTRTHTGEKPFQCRICMRNFSDPSN | |
| | LRRHLRTHLRDEFPTMVFPSGQISQASALAPAPPQVLPQAPAPAPAPAMVSALAQ | |
| | APAPVPVLAPGPPQAVAPPAPKPTQAGEGTLSEALLQLQFDDEDLGALLGNSTDP | |
| | AVFTDLASVDNSEFQQLLNQGIPVAPHTTEPMLMEYPEAITRLVTGAQRPPDPAP | |
| | APLGAPGLPNGLLSGDEDFSSIADMDFSALLSQISSSDTLFTMTQSGPSPLQLPPED | |
| | AYVGNADMIQPDLTPLQPSLDDFMDISDFFTN | |
| pDP1674 | MALPVTALLLPLALLLHAARPEQKLISEEDLDIQMTQTTSSLSASLGDRVTISCRAS | 106 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHGQL | |
| | WFPPKKKRKVSRPGERPFQCRICMRNFSQQTNLTRHTRTHTGEKPFQCRICMRNF | |
| | SQKANLGVHLRTHTGSQKPFQCRICMRNFSDHSSLKRHTRTHTGEKPFQCRICMR | |
| | NFSRADMLRRHLRTHTGSQKPFQCRICMRNFSEKQNLARHTRTHTGEKPFQCRIC | |
| | MRNFSDPSNLRRHLRTHLRDEFPTMVFPSGQISQASALAPAPPQVLPQAPAPAPAP | |
| | AMVSALAQAPAPVPVLAPGPPQAVAPPAPKPTQAGEGTLSEALLQLQFDDEDLG | |
| | ALLGNSTDPAVFTDLASVDNSEFQQLLNQGIPVAPHTTEPMLMEYPEAITRLVTG | |
| | AQRPPDPAPAPLGAPGLPNGLLSGDEDFSSIADMDFSALLSQISSSDTLFTMTQSGP | |
| | SPLQLPPEDAYVGNADMIQPDLTPLQPSLDDFMDISDFFTN | |
| pDP1675 | MALPVTALLLPLALLLHAARPEQKLISEEDLDIQMTQTTSSLSASLGDRVTISCRAS | 107 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSKRKRKHGSLW | |
| | LPPKKKRKVSRPGERPFQCRICMRNFSQQTNLTRHTRTHTGEKPFQCRICMRNFSQ | |
| | KANLGVHLRTHTGSQKPFQCRICMRNFSDHSSLKRHTRTHTGEKPFQCRICMRNF | |
| | SRADMLRRHLRTHTGSQKPFQCRICMRNFSEKQNLARHTRTHTGEKPFQCRICMR | |
| | NFSDPSNLRRHLRTHLRDEFPTMVFPSGQISQASALAPAPPQVLPQAPAPAPAPAM | |
| | VSALAQAPAPVPVLAPGPPQAVAPPAPKPTQAGEGTLSEALLQLQFDDEDLGALL | |
| | GNSTDPAVFTDLASVDNSEFQQLLNQGIPVAPHTTEPMLMEYPEAITRLVTGAQR | |
| | PPDPAPAPLGAPGLPNGLLSGDEDFSSIADMDFSALLSQISSSDTLFTMTQSGPSPL | |
| | QLPPEDAYVGNADMIQPDLTPLQPSLDDFMDISDFFTN | |
| pDP1757 | MALPVTALLLPLALLLHAARPEQKLISEEDLDIQMTQTTSSLSASLGDRVTISCRAS | 108 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRRKKSRPGERP | |
| | FQCRICMRNFSQQTNLTRHTRTHTGEKPFQCRICMRNFSQKANLGVHLRTHTGSQ | |
| | KPFQCRICMRNFSDHSSLKRHTRTHTGEKPFQCRICMRNFSRADMLRRHLRTHTG | |
| | SQKPFQCRICMRNFSEKQNLARHTRTHTGEKPFQCRICMRNFSDPSNLRRHLRTH | |
| | LRDEFPTMVFPSGQISQASALAPAPPQVLPQAPAPAPAPAMVSALAQAPAPVPVL | |
| | APGPPQAVAPPAPKPTQAGEGTLSEALLQLQFDDEDLGALLGNSTDPAVFTDLAS | |
| | VDNSEFQQLLNQGIPVAPHTTEPMLMEYPEAITRLVTGAQRPPDPAPAPLGAPGLP | |
| | NGLLSGDEDFSSIADMDFSALLSQISSSDTLFTMTQSGPSPLQLPPEDAYVGNADM | |
| | IQPDLTPLQPSLDDFMDISDFFTN | |
| pDP1758 | MALPVTALLLPLALLLHAARPEQKLISEEDLDIQMTQTTSSLSASLGDRVTISCRAS | 109 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRRRKKSRPGER | |
| | PFQCRICMRNFSQQTNLTRHTRTHTGEKPFQCRICMRNFSQKANLGVHLRTHTGS | |
| | QKPFQCRICMRNFSDHSSLKRHTRTHTGEKPFQCRICMRNFSRADMLRRHLRTHT | |
| | GSQKPFQCRICMRNFSEKQNLARHTRTHTGEKPFQCRICMRNFSDPSNLRRHLRT | |
| | HLRDEFPTMVFPSGQISQASALAPAPPQVLPQAPAPAPAPAMVSALAQAPAPVPV | |
| | LAPGPPQAVAPPAPKPTQAGEGTLSEALLQLQFDDEDLGALLGNSTDPAVFTDLA | |
| | SVDNSEFQQLLNQGIPVAPHTTEPMLMEYPEAITRLVTGAQRPPDPAPAPLGAPGL | |
| | PNGLLSGDEDFSSIADMDFSALLSQISSSDTLFTMTQSGPSPLQLPPEDAYVGNAD | |
| | MIQPDLTPLQPSLDDFMDISDFFTN | |
| pDP1759 | MALPVTALLLPLALLLHAARPEQKLISEEDLDIQMTQTTSSLSASLGDRVTISCRAS | 110 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRKKSRPGE | |
| | RPFQCRICMRNFSQQTNLTRHTRTHTGEKPFQCRICMRNFSQKANLGVHLRTHTG | |
| | SQKPFQCRICMRNFSDHSSLKRHTRTHTGEKPFQCRICMRNFSRADMLRRHLRTH | |
| | TGSQKPFQCRICMRNFSEKQNLARHTRTHTGEKPFQCRICMRNFSDPSNLRRHLR | |
| | THLRDEFPTMVFPSGQISQASALAPAPPQVLPQAPAPAPAPAMVSALAQAPAPVP | |
| | VLAPGPPQAVAPPAPKPTQAGEGTLSEALLQLQFDDEDLGALLGNSTDPAVFTDL | |
| | ASVDNSEFQQLLNQGIPVAPHTTEPMLMEYPEAITRLVTGAQRPPDPAPAPLGAPG | |
| | LPNGLLSGDEDFSSIADMDFSALLSQISSSDTLFTMTQSGPSPLQLPPEDAYVGNAD | |
| | MIQPDLTPLQPSLDDFMDISDFFTN | |
| pDP1760 | MALPVTALLLPLALLLHAARPEQKLISEEDLDIQMTQTTSSLSASLGDRVTISCRAS | 111 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRKKSRPG | |
| | ERPFQCRICMRNFSQQTNLTRHTRTHTGEKPFQCRICMRNFSQKANLGVHLRTHT | |
| | GSQKPFQCRICMRNFSDHSSLKRHTRTHTGEKPFQCRICMRNFSRADMLRRHLRT | |
| | HTGSQKPFQCRICMRNFSEKQNLARHTRTHTGEKPFQCRICMRNFSDPSNLRRHL | |
| | RTHLRDEFPTMVFPSGQISQASALAPAPPQVLPQAPAPAPAPAMVSALAQAPAPV | |
| | PVLAPGPPQAVAPPAPKPTQAGEGTLSEALLQLQFDDEDLGALLGNSTDPAVFTD | |
| | LASVDNSEFQQLLNQGIPVAPHTTEPMLMEYPEAITRLVTGAQRPPDPAPAPLGAP | |
| | GLPNGLLSGDEDFSSIADMDFSALLSQISSSDTLFTMTQSGPSPLQLPPEDAYVGNA | |
| | DMIQPDLTPLQPSLDDFMDISDFFTN | |
| pDP1761 | MALPVTALLLPLALLLHAARPEQKLISEEDLDIQMTQTTSSLSASLGDRVTISCRAS | 112 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRRKKSRP | |
| | GERPFQCRICMRNFSQQTNLTRHTRTHTGEKPFQCRICMRNFSQKANLGVHLRTH | |
| | TGSQKPFQCRICMRNFSDHSSLKRHTRTHTGEKPFQCRICMRNFSRADMLRRHLR | |
| | THTGSQKPFQCRICMRNFSEKQNLARHTRTHTGEKPFQCRICMRNFSDPSNLRRH | |
| | LRTHLRDEFPTMVFPSGQISQASALAPAPPQVLPQAPAPAPAPAMVSALAQAPAP | |
| | VPVLAPGPPQAVAPPAPKPTQAGEGTLSEALLQLQFDDEDLGALLGNSTDPAVFT | |
| | DLASVDNSEFQQLLNQGIPVAPHTTEPMLMEYPEAITRLVTGAQRPPDPAPAPLG | |
| | APGLPNGLLSGDEDFSSIADMDFSALLSQISSSDTLFTMTQSGPSPLQLPPEDAYVG | |
| | NADMIQPDLTPLQPSLDDFMDISDFFTN | |
| pDP1762 | MALPVTALLLPLALLLHAARPEQKLISEEDLDIQMTQTTSSLSASLGDRVTISCRAS | 113 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQRRKKS | |
| | RPGERPFQCRICMRNFSQQTNLTRHTRTHTGEKPFQCRICMRNFSQKANLGVHLR | |
| | THTGSQKPFQCRICMRNFSDHSSLKRHTRTHTGEKPFQCRICMRNFSRADMLRRH | |
| | LRTHTGSQKPFQCRICMRNFSEKQNLARHTRTHTGEKPFQCRICMRNFSDPSNLR | |
| | RHLRTHLRDEFPTMVFPSGQISQASALAPAPPQVLPQAPAPAPAPAMVSALAQAP | |
| | APVPVLAPGPPQAVAPPAPKPTQAGEGTLSEALLQLQFDDEDLGALLGNSTDPAV | |
| | FTDLASVDNSEFQQLLNQGIPVAPHTTEPMLMEYPEAITRLVTGAQRPPDPAPAPL | |
| | GAPGLPNGLLSGDEDFSSIADMDFSALLSQISSSDTLFTMTQSGPSPLQLPPEDAYV | |
| | GNADMIQPDLTPLQPSLDDFMDISDFFTN | |
| pDP1763 | MALPVTALLLPLALLLHAARPEQKLISEEDLDIQMTQTTSSLSASLGDRVTISCRAS | 114 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHRRK | |
| | KSRPGERPFQCRICMRNFSQQTNLTRHTRTHTGEKPFQCRICMRNFSQKANLGVH | |
| | LRTHTGSQKPFQCRICMRNFSDHSSLKRHTRTHTGEKPFQCRICMRNFSRADMLR | |
| | RHLRTHTGSQKPFQCRICMRNFSEKQNLARHTRTHTGEKPFQCRICMRNFSDPSN | |
| | LRRHLRTHLRDEFPTMVFPSGQISQASALAPAPPQVLPQAPAPAPAPAMVSALAQ | |
| | APAPVPVLAPGPPQAVAPPAPKPTQAGEGTLSEALLQLQFDDEDLGALLGNSTDP | |
| | AVFTDLASVDNSEFQQLLNQGIPVAPHTTEPMLMEYPEAITRLVTGAQRPPDPAP | |
| | APLGAPGLPNGLLSGDEDFSSIADMDFSALLSQISSSDTLFTMTQSGPSPLQLPPED | |
| | AYVGNADMIQPDLTPLQPSLDDFMDISDFFTN | |
| pDP1764 | MALPVTALLLPLALLLHAARPEQKLISEEDLDIQMTQTTSSLSASLGDRVTISCRAS | 115 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHGRR | |
| | KKSRPGERPFQCRICMRNFSQQTNLTRHTRTHTGEKPFQCRICMRNFSQKANLGV | |
| | HLRTHTGSQKPFQCRICMRNFSDHSSLKRHTRTHTGEKPFQCRICMRNFSRADML | |
| | RRHLRTHTGSQKPFQCRICMRNFSEKQNLARHTRTHTGEKPFQCRICMRNFSDPS | |
| | NLRRHLRTHLRDEFPTMVFPSGQISQASALAPAPPQVLPQAPAPAPAPAMVSALA | |
| | QAPAPVPVLAPGPPQAVAPPAPKPTQAGEGTLSEALLQLQFDDEDLGALLGNSTD | |
| | PAVFTDLASVDNSEFQQLLNQGIPVAPHTTEPMLMEYPEAITRLVTGAQRPPDPAP | |
| | APLGAPGLPNGLLSGDEDFSSIADMDFSALLSQISSSDTLFTMTQSGPSPLQLPPED | |
| | AYVGNADMIQPDLTPLQPSLDDFMDISDFFTN | |
| pDP1765 | MALPVTALLLPLALLLHAARPEQKLISEEDLDIQMTQTTSSLSASLGDRVTISCRAS | 116 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHGQR | |
| | RKKSRPGERPFQCRICMRNFSQQTNLTRHTRTHTGEKPFQCRICMRNFSQKANLG | |
| | VHLRTHTGSQKPFQCRICMRNFSDHSSLKRHTRTHTGEKPFQCRICMRNFSRADM | |
| | LRRHLRTHTGSQKPFQCRICMRNFSEKQNLARHTRTHTGEKPFQCRICMRNFSDP | |
| | SNLRRHLRTHLRDEFPTMVFPSGQISQASALAPAPPQVLPQAPAPAPAPAMVSAL | |
| | AQAPAPVPVLAPGPPQAVAPPAPKPTQAGEGTLSEALLQLQFDDEDLGALLGNST | |
| | DPAVFTDLASVDNSEFQQLLNQGIPVAPHTTEPMLMEYPEAITRLVTGAQRPPDP | |
| | APAPLGAPGLPNGLLSGDEDFSSIADMDFSALLSQISSSDTLFTMTQSGPSPLQLPP | |
| | EDAYVGNADMIQPDLTPLQPSLDDFMDISDFFTN | |
| pDP1766 | MALPVTALLLPLALLLHAARPEQKLISEEDLDIQMTQTTSSLSASLGDRVTISCRAS | 117 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHGQLR | |
| | RKKSRPGERPFQCRICMRNFSQQTNLTRHTRTHTGEKPFQCRICMRNFSQKANLG | |
| | VHLRTHTGSQKPFQCRICMRNFSDHSSLKRHTRTHTGEKPFQCRICMRNFSRADM | |
| | LRRHLRTHTGSQKPFQCRICMRNFSEKQNLARHTRTHTGEKPFQCRICMRNFSDP | |
| | SNLRRHLRTHLRDEFPTMVFPSGQISQASALAPAPPQVLPQAPAPAPAPAMVSAL | |
| | AQAPAPVPVLAPGPPQAVAPPAPKPTQAGEGTLSEALLQLQFDDEDLGALLGNST | |
| | DPAVFTDLASVDNSEFQQLLNQGIPVAPHTTEPMLMEYPEAITRLVTGAQRPPDP | |
| | APAPLGAPGLPNGLLSGDEDFSSIADMDFSALLSQISSSDTLFTMTQSGPSPLQLPP | |
| | EDAYVGNADMIQPDLTPLQPSLDDFMDISDFFTN | |
| pDP1767 | MALPVTALLLPLALLLHAARPEQKLISEEDLDIQMTQTTSSLSASLGDRVTISCRAS | 118 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHGQL | |
| | WRRKKSRPGERPFQCRICMRNFSQQTNLTRHTRTHTGEKPFQCRICMRNFSQKAN | |
| | LGVHLRTHTGSQKPFQCRICMRNFSDHSSLKRHTRTHTGEKPFQCRICMRNFSRA | |
| | DMLRRHLRTHTGSQKPFQCRICMRNFSEKQNLARHTRTHTGEKPFQCRICMRNFS | |
| | DPSNLRRHLRTHLRDEFPTMVFPSGQISQASALAPAPPQVLPQAPAPAPAPAMVSA | |
| | LAQAPAPVPVLAPGPPQAVAPPAPKPTQAGEGTLSEALLQLQFDDEDLGALLGNS | |
| | TDPAVFTDLASVDNSEFQQLLNQGIPVAPHTTEPMLMEYPEAITRLVTGAQRPPDP | |
| | APAPLGAPGLPNGLLSGDEDFSSIADMDFSALLSQISSSDTLFTMTQSGPSPLQLPP | |
| | EDAYVGNADMIQPDLTPLQPSLDDFMDISDFFTN | |
| pDP1768 | MALPVTALLLPLALLLHAARPEQKLISEEDLDIQMTQTTSSLSASLGDRVTISCRAS | 119 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHGQL | |
| | WFRRKKSRPGERPFQCRICMRNFSQQTNLTRHTRTHTGEKPFQCRICMRNFSQKA | |
| | NLGVHLRTHTGSQKPFQCRICMRNFSDHSSLKRHTRTHTGEKPFQCRICMRNFSR | |
| | ADMLRRHLRTHTGSQKPFQCRICMRNFSEKQNLARHTRTHTGEKPFQCRICMRNF | |
| | SDPSNLRRHLRTHLRDEFPTMVFPSGQISQASALAPAPPQVLPQAPAPAPAPAMVS | |
| | ALAQAPAPVPVLAPGPPQAVAPPAPKPTQAGEGTLSEALLQLQFDDEDLGALLGN | |
| | STDPAVFTDLASVDNSEFQQLLNQGIPVAPHTTEPMLMEYPEAITRLVTGAQRPPD | |
| | PAPAPLGAPGLPNGLLSGDEDFSSIADMDFSALLSQISSSDTLFTMTQSGPSPLQLP | |
| | PEDAYVGNADMIQPDLTPLQPSLDDFMDISDFFTN | |
| pDP1725 | MALPVTALLLPLALLLHAARPEQKLISEEDLDIQMTQTTSSLSASLGDRVTISCRAS | 120 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHGQL | |
| | WFPRRKKSRPGERPFQCRICMRNFSQQTNLTRHTRTHTGEKPFQCRICMRNFSQK | |
| | ANLGVHLRTHTGSQKPFQCRICMRNFSDHSSLKRHTRTHTGEKPFQCRICMRNFS | |
| | RADMLRRHLRTHTGSQKPFQCRICMRNFSEKQNLARHTRTHTGEKPFQCRICMR | |
| | NFSDPSNLRRHLRTHLRDEFPTMVFPSGQISQASALAPAPPQVLPQAPAPAPAPAM | |
| | VSALAQAPAPVPVLAPGPPQAVAPPAPKPTQAGEGTLSEALLQLQFDDEDLGALL | |
| | GNSTDPAVFTDLASVDNSEFQQLLNQGIPVAPHTTEPMLMEYPEAITRLVTGAQR | |
| | PPDPAPAPLGAPGLPNGLLSGDEDFSSIADMDFSALLSQISSSDTLFTMTQSGPSPL | |
| | QLPPEDAYVGNADMIQPDLTPLQPSLDDFMDISDFFTN | |
| pDP1769 | MALPVTALLLPLALLLHAARPEQKLISEEDLDIQMTQTTSSLSASLGDRVTISCRAS | 121 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHGQL | |
| | WFPERRKKSRPGERPFQCRICMRNFSQQTNLTRHTRTHTGEKPFQCRICMRNFSQ | |
| | KANLGVHLRTHTGSQKPFQCRICMRNFSDHSSLKRHTRTHTGEKPFQCRICMRNF | |
| | SRADMLRRHLRTHTGSQKPFQCRICMRNFSEKQNLARHTRTHTGEKPFQCRICMR | |
| | NFSDPSNLRRHLRTHLRDEFPTMVFPSGQISQASALAPAPPQVLPQAPAPAPAPAM | |
| | VSALAQAPAPVPVLAPGPPQAVAPPAPKPTQAGEGTLSEALLQLQFDDEDLGALL | |
| | GNSTDPAVFTDLASVDNSEFQQLLNQGIPVAPHTTEPMLMEYPEAITRLVTGAQR | |
| | PPDPAPAPLGAPGLPNGLLSGDEDFSSIADMDFSALLSQISSSDTLFTMTQSGPSPL | |
| | QLPPEDAYVGNADMIQPDLTPLQPSLDDFMDISDFFTN | |
| pDP1770 | MALPVTALLLPLALLLHAARPEQKLISEEDLDIQMTQTTSSLSASLGDRVTISCRAS | 122 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHGQL | |
| | WFPEGRRKKSRPGERPFQCRICMRNFSQQTNLTRHTRTHTGEKPFQCRICMRNFS | |
| | QKANLGVHLRTHTGSQKPFQCRICMRNFSDHSSLKRHTRTHTGEKPFQCRICMRN | |
| | FSRADMLRRHLRTHTGSQKPFQCRICMRNFSEKQNLARHTRTHTGEKPFQCRICM | |
| | RNFSDPSNLRRHLRTHLRDEFPTMVFPSGQISQASALAPAPPQVLPQAPAPAPAPA | |
| | MVSALAQAPAPVPVLAPGPPQAVAPPAPKPTQAGEGTLSEALLQLQFDDEDLGAL | |
| | LGNSTDPAVFTDLASVDNSEFQQLLNQGIPVAPHTTEPMLMEYPEAITRLVTGAQ | |
| | RPPDPAPAPLGAPGLPNGLLSGDEDFSSIADMDFSALLSQISSSDTLFTMTQSGPSP | |
| | LQLPPEDAYVGNADMIQPDLTPLQPSLDDFMDISDFFTN | |
| pDP1771 | MALPVTALLLPLALLLHAARPEQKLISEEDLDIQMTQTTSSLSASLGDRVTISCRAS | 123 |
| | QDISKYLNWYQQKPDGTVKLLIYHTSRLHSGVPSRFSGSGSGTDYSLTISNLEQED | |
| | IATYFCQQGNTLPYTFGGGTKLEITGGGGSGGGGSGGGGSEVKLQESGPGLVAPS | |
| | QSLSVTCTVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSSTTTP | |
| | APRPPTPAPTIASQPLSLRPEACFMYVAAAAFVLLFFVGCGVLLSRKRRRQHGQL | |
| | WFPEGFRRKKSRPGERPFQCRICMRNFSQQTNLTRHTRTHTGEKPFQCRICMRNFS | |
| | QKANLGVHLRTHTGSQKPFQCRICMRNFSDHSSLKRHTRTHTGEKPFQCRICMRN | |
| | FSRADMLRRHLRTHTGSQKPFQCRICMRNFSEKQNLARHTRTHTGEKPFQCRICM | |
| | RNFSDPSNLRRHLRTHLRDEFPTMVFPSGQISQASALAPAPPQVLPQAPAPAPAPA | |
| | MVSALAQAPAPVPVLAPGPPQAVAPPAPKPTQAGEGTLSEALLQLQFDDEDLGAL | |
| | LGNSTDPAVFTDLASVDNSEFQQLLNQGIPVAPHTTEPMLMEYPEAITRLVTGAQ | |
| | RPPDPAPAPLGAPGLPNGLLSGDEDFSSIADMDFSALLSQISSSDTLFTMTQSGPSP | |
| | LQLPPEDAYVGNADMIQPDLTPLQPSLDDFMDISDFFTN | |

1. A chimeric receptor comprising, from N-terminus to C-terminus:
a. an extracellular ligand-binding domain having a binding affinity for a ligand;
b. a linking polypeptide capable of promoting oligomer formation of the chimeric polypeptide via intermolecular disulfide bonding;
c. a transmembrane domain comprising one or more ligand-inducible proteolytic cleavage sites; and
d. an intracellular domain comprising two or more human or humanized transcriptional regulators,
wherein binding of the ligand to the extracellular ligand-binding domain induces cleavage at the one or more ligand-inducible proteolytic cleavage sites between the transcriptional regulator and the linking polypeptide, and
wherein the chimeric polypeptide does not comprise a LIN-12-Notch repeat (LNR) and/or a heterodimerization domain (HD) of a Notch receptor.

2. The chimeric receptor of claim 1, wherein the extracellular ligand-binding domain comprises an antigen-binding moiety capable of binding to the ligand on the surface of a cell, wherein the cell is a pathogenic cell, a human cell, a tumor cell, or a terminally differentiated cell.

3-6. (canceled)

7. The chimeric receptor of claim 1, wherein the ligand:
(i) comprises a protein or a carbohydrate,
(ii) is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), blue fluorescent protein (BFP) enhanced green fluorescent protein (EGFP), and signal regulatory protein α (SIRPα);
iii) is selected from cell surface receptors, adhesion proteins, integrins, mucins, lectins, tumor associated antigens, and tumor-specific antigens; and/or
iv) is a tumor-associated antigen or a tumor-specific antigen.

8-10. (canceled)

11. The chimeric receptor of claim 1,
wherein the extracellular ligand-binding domain comprises the ligand-binding portion of a receptor.

12. The chimeric receptor of claim 2, wherein the antigen-binding moiety,
(i) is selected from the group consisting of an antibody, a nanobody, a diabody, a triabody, a minibody, an F(ab′)2 fragment, an F(ab)v fragment, a single chain variable fragment (scFv), a single domain antibody (sdAb), and a functional fragment thereof; and/or
(ii) specifically binds to a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD117), MUC1, NCAM, PAP, PDGFR-β, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

13-16. (canceled)

17. The chimeric receptor of claim 1,
wherein the linking polypeptide comprises a hinge domain.

18-20. (canceled)

21. The chimeric receptor of claim 1,
wherein the one or more ligand-inducible proteolytic cleavage sites comprises a gamma secretase cleavage site.

22. The chimeric receptor of claim 1, wherein the transmembrane domain comprises an amino acid sequence having at least 80% sequence identity to SEQ ID NO: 4.

23. The chimeric receptor of claim 1, wherein the transmembrane domain further comprises a stop-transfer-sequence.

24-26. (canceled)

27. The chimeric receptor of claim 1, further comprising (i) an intracellular signaling domain (SD) derived from a signaling molecule and (ii) an activation domain, and optionally wherein the signaling molecule comprises a class 1 or a class 3 human membrane protein.

28. (canceled)

29. The chimeric receptor of claim 27, wherein the signaling molecule is selected from the list consisting of OX40, ICOS, 4-1BB, CTLA4, CD28, CD30, CD2, CD27, and CD226.

30. The chimeric receptor of claim 27, wherein the activation domain comprises one or more immunoreceptor tyrosine-based activation motifs (ITAMs).

31-33. (canceled)

34. The chimeric receptor of claim 1, wherein the two or more human or humanized transcriptional regulators comprise a transcriptional activator, or a transcriptional repressor.

35. (canceled)

36. The chimeric polypeptide of claim 1, wherein the intracellular domain further comprises a DNA-binding domain sequence derived from a protein selected from the group consisting of Gal4, tetR, ZFHD1, Zif268, HAP1, ZF3, ZF4, ZF6, ZF10, and ZF11.

37. The chimeric polypeptide of claim 1, wherein the two or more human or humanized transcriptional regulators comprise a TAD derived from the group consisting of HSF-1, GATA3, HIF1a, GR Tau1, ATF6, ELF3, p53, MIER3, MLXIPL, NFE2L1, and PTF1A.

38. The chimeric receptor of claim 1, comprising an amino acid sequence having at least 80% sequence identity to any one of SEQ ID NOs: 35-71 and 99-123.

39. (canceled)

40. A recombinant nucleic acid comprising a nucleotide sequence encoding the chimeric receptor of claim 1.

41-43. (canceled)

44. A recombinant cell comprising,
i. the chimeric receptor of claim 1; and/or
ii. a recombinant nucleic comprising a nucleotide sequence encoding the chimeric receptor of (a).

45-54. (canceled)

55. A method for making a recombinant cell, the method comprising:
a) providing a cell capable of protein expression; and
b) contacting the provided cell with a recombinant nucleic acid of claim 40.

56-57. (canceled)

58. A pharmaceutical composition comprising a pharmaceutically acceptable carrier, and one or more of the following:
a) a chimeric receptor according to claim 1:
b) a recombinant nucleic acid comprising a nucleotide sequence encoding the chimeric receptor of (a); and
c) a recombinant cell comprising the chimeric receptor of (i) and/or the recombinant nucleic acid of (b).

59-60. (canceled)

61. A system for modulating an activity of a cell, inhibiting a target cancer cell, or treating a health condition in an individual in need thereof, the system comprising one or more of the following:
i. a chimeric receptor according to claim 1;
ii. a recombinant nucleic acid comprising a nucleotide sequence encoding the chimeric receptor of (a);
iii. a recombinant cell comprising the chimeric receptor of (a) and/or the nucleic acid of (b); and
iv. a pharmaceutical composition comprising a pharmaceutically acceptable carrier and one or more of the following:
1. the chimeric receptor of (a),
2. the recombinant nucleic acid of (b), and
3. the recombinant cell of (c).

62. A method for modulating an activity of a cell, the method comprising:
a) providing a recombinant cell according to claim 44; and
b) contacting the recombinant cell with a selected ligand, wherein binding of the selected ligand to the extracellular ligand-binding domain induces cleavage of a ligand-inducible proteolytic cleavage site and releases the transcriptional regulator, wherein the released transcriptional regulator modulates an activity of the recombinant cell.

63-68. (canceled)

69. A method for inhibiting an activity of a target cell in an individual, the method comprising administering to the individual an effective number of the recombinant cells according to claim 44, wherein the recombinant cells inhibit an activity of the target cell in the individual.

70-72. (canceled)

73. A method for the treatment of a health condition in an individual in need thereof, the method comprising administering to the individual a first therapy comprising an effective number of the recombinant cell according to claim 44, wherein the recombinant cell treats the health condition in the individual.

74-85. (canceled)


SEQUENCE LISTING (155 sequences)

SEQ ID NO: 1 - protein, 273 aa, Artificial sequence
  Synthetic polypeptide
  MISC_FEATURE: ECD (for CD19)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 277 aa, Artificial sequence
  Synthetic polypeptide
  MISC_FEATURE: ECD (for APPL2)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVL

SEQ ID NO: 3 - protein, 27 aa, Artificial sequence
  Synthetic polypeptide
  MISC_FEATURE: Linking polypeptide
TTTPAPRPPTPAPTIASQPLSLRPEAC

SEQ ID NO: 4 - protein, 22 aa, Artificial sequence
  Synthetic polypeptide
  MISC_FEATURE: TMD
FMYVAAAAFVLLFFVGCGVLLS

SEQ ID NO: 5 - protein, 6 aa, Artificial sequence
  Synthetic polypeptide
  MISC_FEATURE: STS
KRKRKH

SEQ ID NO: 6 - protein, 202 aa, Artificial sequence
  Synthetic polypeptide
  MISC_FEATURE: DBD (ZF3)
APKKKRKVGIHGVPGGLEGGGGSGGTASSRPGERPFQCRICMRNFSQRSSLVRHTRTHTGEKPFQCRICMRNFSDKSVLARHLRTHTGSQKPFQCRICMRNFSQRSSLVRHTRTHTGEKPFQCRICMRNFSQRNNLGRHLRTHTGSQKPFQCRICMRNFSTHAVLTRHTRTHTGEKPFQCRICMRNFSDRGNLTRHLRTHLR

SEQ ID NO: 7 - protein, 202 aa, Artificial sequence
  Synthetic polypeptide
  MISC_FEATURE: DBD (ZF6)
APKKKRKVGIHGVPGGLEGGGGSGGTASSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLR

SEQ ID NO: 8 - protein, 181 aa, Artificial sequence
  Synthetic polypeptide
  MISC_FEATURE: DBD (ZF6/SV40NLS)
PKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLR

SEQ ID NO: 9 - protein, 178 aa, Artificial sequence
  Synthetic polypeptide
  MISC_FEATURE: DBD (ZF6/Notch1NLS)
RRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLR

SEQ ID NO: 10 - protein, 181 aa, Artificial sequence
  Synthetic polypeptide
  MISC_FEATURE: DBD (ZF10/SV40NLS)
PKKKRKVSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLR

SEQ ID NO: 11 - protein, 178 aa, Artificial sequence
  Synthetic polypeptide
  MISC_FEATURE: DBD (ZF10/Notch1NLS)
RRKKSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLR

SEQ ID NO: 12 - protein, 139 aa, Artificial sequence
  Synthetic polypeptide
  MISC_FEATURE: DBD (PAX6)
MQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGA

SEQ ID NO: 13 - protein, 5 aa, Artificial sequence
  Synthetic polypeptide
  MISC_FEATURE: Linker - 1
GSTCR

SEQ ID NO: 14 - protein, 1 aa, Artificial sequence
  Synthetic polypeptide
  MISC_FEATURE: Linker - 1 variant
M

SEQ ID NO: 15 - protein, 181 aa, Artificial sequence
  Synthetic polypeptide
  MISC_FEATURE: TAD (mouse p65)
PSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISS

SEQ ID NO: 16 - protein, 183 aa, Artificial sequence
  Synthetic polypeptide
  MISC_FEATURE: TAD (human p65 (mouse-homologous region))
PSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 17 - protein, 191 aa, Artificial sequence
  Synthetic polypeptide
  MISC_FEATURE: TAD (human p65)
DEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 18 - protein, 124 aa, Artificial sequence
  Synthetic polypeptide
  MISC_FEATURE: TAD (human p65, minimized TAD1 and TAD2)
PTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 19 - protein, 31 aa, Artificial sequence
  Synthetic polypeptide
  MISC_FEATURE: TAD (human p65, minimized TAD1)
PGLPNGLLSGDEDFSSIADMDFSALLSQISS

SEQ ID NO: 20 - protein, 58 aa, Artificial sequence
  Synthetic polypeptide
  MISC_FEATURE: TAD (human GR Tau1, minimal)
DQSTFDILQDLEFSSGSPGKETNESPWRSDLLIDENCLLSPLAGEDDSFLLEGNSNED

SEQ ID NO: 21 - protein, 45 aa, Artificial sequence
  Synthetic polypeptide
  MISC_FEATURE: TAD (human p53, TAD2)
DDLMLSPDDIEQWFTEDPGPDEAPRMPEAAPPVAPAPAAPTPAAP

SEQ ID NO: 22 - protein, 32 aa, Artificial sequence
  Synthetic polypeptide
  MISC_FEATURE: TAD (human ATF6 8aa fragment (4x repeats))
DFDLDLLPDFDLDLLPDFDLDLLPDFDLDLLP

SEQ ID NO: 23 - protein, 8 aa, Artificial sequence
  Synthetic polypeptide
  MISC_FEATURE: Linker -2
SGQGGGGS

SEQ ID NO: 24 - protein, 124 aa, Artificial sequence
  Synthetic polypeptide
  MISC_FEATURE: Additional Transcriptional Effector (human HSF1 TAD)
GFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 25 - protein, 45 aa, Artificial sequence
  Synthetic polypeptide
  MISC_FEATURE: Additional Transcriptional Effector (human GATA3, TAD1)
GLSHSYMDAAQYPLPEEVDVLFNIDGQGNHVPPYYGNSVRATVQR

SEQ ID NO: 26 - protein, 39 aa, Artificial sequence
  Synthetic polypeptide
  MISC_FEATURE: Additional Transcriptional Effector (human p53, TAD1)
EEPQSDPSVEPPLSQETFSDLWKLLPENNVLSPLPSQAM

SEQ ID NO: 27 - protein, 45 aa, Artificial sequence; identical to SEQ ID NO: 21
  Synthetic polypeptide
  MISC_FEATURE: Additional Transcriptional Effector (human p53, TAD2)

SEQ ID NO: 28 - protein, 41 aa, Artificial sequence
  Synthetic polypeptide
  MISC_FEATURE: Additional Transcriptional Effector (human HIF1a, CAD)
SMDESGLPQLTSYDCEVNAPIQGSRNLLQGEELLRALDQVN

SEQ ID NO: 29 - protein, 58 aa, Artificial sequence; identical to SEQ ID NO: 20
  Synthetic polypeptide
  MISC_FEATURE: Additional Transcriptional Effector (human GR Tau1 (minimal), TAD)

SEQ ID NO: 30 - protein, 35 aa, Artificial sequence
  Synthetic polypeptide
  MISC_FEATURE: Additional Transcriptional Effector (human ATF6 7aa , TAD, 5x repeats)
DFDLDLLDFDLDLLDFDLDLLDFDLDLLDFDLDLL

SEQ ID NO: 31 - protein, 62 aa, Artificial sequence
  Synthetic polypeptide
  MISC_FEATURE: Additional Transcriptional Effector (human ELF3, TAD, 2x repeats)
TSSSSDELSWIIELLEKDGMAFQEALDPGPFTSSSSDELSWIIELLEKDGMAFQEALDPGPF

SEQ ID NO: 32 - protein, 45 aa, Artificial sequence; identical to SEQ ID NO: 21
  Synthetic polypeptide
  MISC_FEATURE: Additional Transcriptional Effector (human p53, TAD2)

SEQ ID NO: 33 - protein, 124 aa, Artificial sequence; identical to SEQ ID NO: 18
  Synthetic polypeptide
  MISC_FEATURE: Additional Transcriptional Effector (human p65 (428))

SEQ ID NO: 34 - protein, 191 aa, Artificial sequence; identical to SEQ ID NO: 17
  Synthetic polypeptide
  MISC_FEATURE: Additional Transcriptional Effector (human p65 (361))

SEQ ID NO: 35 - protein, 716 aa, Artificial sequence
  Synthetic polypeptide
  MISC_FEATURE: FULL SEQUENCE (pDP1179)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQRSSLVRHTRTHTGEKPFQCRICMRNFSDKSVLARHLRTHTGSQKPFQCRICMRNFSQRSSLVRHTRTHTGEKPFQCRICMRNFSQRNNLGRHLRTHTGSQKPFQCRICMRNFSTHAVLTRHTRTHTGEKPFQCRICMRNFSDRGNLTRHLRTHLRGSTCRPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISS

SEQ ID NO: 36 - protein, 718 aa, Artificial sequence
  Synthetic polypeptide
  MISC_FEATURE: FULL SEQUENCE (pDP1180)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQRSSLVRHTRTHTGEKPFQCRICMRNFSDKSVLARHLRTHTGSQKPFQCRICMRNFSQRSSLVRHTRTHTGEKPFQCRICMRNFSQRNNLGRHLRTHTGSQKPFQCRICMRNFSTHAVLTRHTRTHTGEKPFQCRICMRNFSDRGNLTRHLRTHLRGSTCRPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 37 - protein, 858 aa, Artificial sequence
  Synthetic polypeptide
  MISC_FEATURE: FULL SEQUENCE (pDP1181)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQRSSLVRHTRTHTGEKPFQCRICMRNFSDKSVLARHLRTHTGSQKPFQCRICMRNFSQRSSLVRHTRTHTGEKPFQCRICMRNFSQRNNLGRHLRTHTGSQKPFQCRICMRNFSTHAVLTRHTRTHTGEKPFQCRICMRNFSDRGNLTRHLRTHLRGSTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 38 - protein, 850 aa, Artificial sequence
  Synthetic polypeptide
  MISC_FEATURE: FULL SEQUENCE (pDP1182)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQRSSLVRHTRTHTGEKPFQCRICMRNFSDKSVLARHLRTHTGSQKPFQCRICMRNFSQRSSLVRHTRTHTGEKPFQCRICMRNFSQRNNLGRHLRTHTGSQKPFQCRICMRNFSTHAVLTRHTRTHTGEKPFQCRICMRNFSDRGNLTRHLRTHLRGSTCRPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 39 - protein, 726 aa, Artificial sequence
  Synthetic polypeptide
  MISC_FEATURE: FULL SEQUENCE (pDP1160)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRGSTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 40 - protein, 848 aa, Artificial sequence
  Synthetic polypeptide
  MISC_FEATURE: FULL SEQUENCE (pDP1174)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRGSTCRPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 41 - protein, 779 aa, Artificial sequence
  Synthetic polypeptide
  MISC_FEATURE: FULL SEQUENCE (pDP1279)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRGSTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSGLSHSYMDAAQYPLPEEVDVLFNIDGQGNHVPPYYGNSVRATVQR

SEQ ID NO: 42 - protein, 773 aa, Artificial sequence
  Synthetic polypeptide
  MISC_FEATURE: FULL SEQUENCE (pDP1280)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRGSTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSEEPQSDPSVEPPLSQETFSDLWKLLPENNVLSPLPSQAM

SEQ ID NO: 43 - protein, 779 aa, Artificial sequence
  Synthetic polypeptide
  MISC_FEATURE: FULL SEQUENCE (pDP1281)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRGSTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSDDLMLSPDDIEQWFTEDPGPDEAPRMPEAAPPVAPAPAAPTPAAP

SEQ ID NO: 44 - protein, 775 aa, Artificial sequence
  Synthetic polypeptide
  MISC_FEATURE: FULL SEQUENCE (pDP1285)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRGSTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSSMDESGLPQLTSYDCEVNAPIQGSRNLLQGEELLRALDQVN

SEQ ID NO: 45 - protein, 792 aa, Artificial sequence
  Synthetic polypeptide
  MISC_FEATURE: FULL SEQUENCE (pDP1286)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRGSTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSDQSTFDILQDLEFSSGSPGKETNESPWRSDLLIDENCLLSPLAGEDDSFLLEGNSNED

SEQ ID NO: 46 - protein, 769 aa, Artificial sequence
  Synthetic polypeptide
  MISC_FEATURE: FULL SEQUENCE (pDP1290)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRGSTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSDFDLDLLDFDLDLLDFDLDLLDFDLDLLDFDLDLL

SEQ ID NO: 47 - protein, 796 aa, Artificial sequence
  Synthetic polypeptide
  MISC_FEATURE: FULL SEQUENCE (pDP1292)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHAPKKKRKVGIHGVPGGLEGGGGSGGTASSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRGSTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSTSSSSDELSWIIELLEKDGMAFQEALDPGPFTSSSSDELSWIIELLEKDGMAFQEALDPGPF

SEQ ID NO: 48 - protein, 704 aa, Artificial sequence
  Synthetic polypeptide
  MISC_FEATURE: FULL SEQUENCE (pDP1338)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 49 - protein, 836 aa, Artificial sequence
  Synthetic polypeptide
  MISC_FEATURE: FULL SEQUENCE (pDP1339)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 50 - protein, 770 aa, Artificial sequence
  Synthetic polypeptide
  MISC_FEATURE: FULL SEQUENCE (pDP1340)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSDQSTFDILQDLEFSSGSPGKETNESPWRSDLLIDENCLLSPLAGEDDSFLLEGNSNED

SEQ ID NO: 51 - protein, 701 aa, Artificial sequence
  Synthetic polypeptide
  MISC_FEATURE: FULL SEQUENCE (pDP1341)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 52 - protein, 833 aa, Artificial sequence
  Synthetic polypeptide
  MISC_FEATURE: FULL SEQUENCE (pDP1342)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 53 - protein, 767 aa, Artificial sequence
  Synthetic polypeptide
  MISC_FEATURE: FULL SEQUENCE (pDP1343)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSDQSTFDILQDLEFSSGSPGKETNESPWRSDLLIDENCLLSPLAGEDDSFLLEGNSNED

SEQ ID NO: 54 - protein, 704 aa, Artificial sequence
  Synthetic polypeptide
  MISC_FEATURE: FULL SEQUENCE (pDP1344)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHPKKKRKVSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 55 - protein, 836 aa, Artificial sequence
  Synthetic polypeptide
  MISC_FEATURE: FULL SEQUENCE (pDP1345)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHPKKKRKVSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 56 - protein, 770 aa, Artificial sequence
  Synthetic polypeptide
  MISC_FEATURE: FULL SEQUENCE (pDP1346)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHPKKKRKVSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSDQSTFDILQDLEFSSGSPGKETNESPWRSDLLIDENCLLSPLAGEDDSFLLEGNSNED

SEQ ID NO: 57 - protein, 701 aa, Artificial sequence
  Synthetic polypeptide
  MISC_FEATURE: FULL SEQUENCE (pDP1347)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHRRKKSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 58 - protein, 833 aa, Artificial sequence
  Synthetic polypeptide
  MISC_FEATURE: FULL SEQUENCE (pDP1348)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHRRKKSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 59 - protein, 767 aa, Artificial sequence
  Synthetic polypeptide
  MISC_FEATURE: FULL SEQUENCE (pDP1349)
MALPVTALLLPLALLLHAARPEQKLISEEDLQVQLQQSGGGLVKPGGSLRLSCAASGFTFSSYAMHWVRQAPGKGLEWVAVISYDGSNKYYADSVKGRFTISRDNSKNTLYLQMDSLRAEDTAVYYCAKEGDSSRWSYDLWGRGTLVTVSSGGGGSGGGGSGGGGSQSALTQPASVSGSPGQSITISCTGTSSDVGGYNYVSWYQQHPGKAPKVMIYDVTNRPSGVSNRFSGSKSGNTASLTISGLQAEDEADYYCSSYTIASTLVVFGGGTKLTVLTTTPAPRPPTPAPTIASQPLSLRPEACFMYVAAAAFVLLFFVGCGVLLSKRKRKHRRKKSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSDQSTFDILQDLEFSSGSPGKETNESPWRSDLLIDENCLLSPLAGEDDSFLLEGNSNED

SEQ ID NO: 60 - protein, 657 aa, Artificial sequence
  Synthetic polypeptide
  MISC_FEATURE: FULL SEQUENCE (pRay451A)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGAPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSDQSTFDILQDLEFSSGSPGKETNESPWRSDLLIDENCLLSPLAGEDDSFLLEGNSNED

SEQ ID NO: 61 - protein, 649 aa, Artificial sequence
  Synthetic polypeptide
  MISC_FEATURE: FULL SEQUENCE (pRay451B)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGAPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSDQSTFDILQDLEFSSGSPGKETNESPWRSDLLIDENCLLSPLAGEDDSFLLEGNSNED

SEQ ID NO: 62 - protein, 724 aa, Artificial sequence
  Synthetic polypeptide
  MISC_FEATURE: FULL SEQUENCE (pRay451D)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGADEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSDQSTFDILQDLEFSSGSPGKETNESPWRSDLLIDENCLLSPLAGEDDSFLLEGNSNED

SEQ ID NO: 63 - protein, 716 aa, Artificial sequence
  Synthetic polypeptide
  MISC_FEATURE: FULL SEQUENCE (pRay451E)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGADEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSDQSTFDILQDLEFSSGSPGKETNESPWRSDLLIDENCLLSPLAGEDDSFLLEGNSNED

SEQ ID NO: 64 - protein, 693 aa, Artificial sequence
  Synthetic polypeptide
  MISC_FEATURE: FULL SEQUENCE (pRay451F)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGADEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSDFDLDLLDFDLDLLDFDLDLLDFDLDLLDFDLDLL

SEQ ID NO: 65 - protein, 626 aa, Artificial sequence
  Synthetic polypeptide
  MISC_FEATURE: FULL SEQUENCE (pRay451G)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGAPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSDFDLDLLDFDLDLLDFDLDLLDFDLDLLDFDLDLL

SEQ ID NO: 66 - protein, 533 aa, Artificial sequence
  Synthetic polypeptide
  MISC_FEATURE: FULL SEQUENCE (pRay451H)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGAPGLPNGLLSGDEDFSSIADMDFSALLSQISSDFDLDLLDFDLDLLDFDLDLLDFDLDLLDFDLDLL

SEQ ID NO: 67 - protein, 711 aa, Artificial sequence
  Synthetic polypeptide
  MISC_FEATURE: FULL SEQUENCE (pRay451I)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGADEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSGQGGGGSDDLMLSPDDIEQWFTEDPGPDEAPRMPEAAPPVAPAPAAPTPAAP

SEQ ID NO: 68 - protein, 649 aa, Artificial sequence
  Synthetic polypeptide
  MISC_FEATURE: FULL SEQUENCE (pRay451J)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHDQSTFDILQDLEFSSGSPGKETNESPWRSDLLIDENCLLSPLAGEDDSFLLEGNSNEDMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGAPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 69 - protein, 637 aa, Artificial sequence
  Synthetic polypeptide
  MISC_FEATURE: FULL SEQUENCE (pRay451K)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMDDLMLSPDDIEQWFTEDPGPDEAPRMPEAAPPVAPAPAAPTPAAPMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGAPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 70 - protein, 623 aa, Artificial sequence
  Synthetic polypeptide
  MISC_FEATURE: FULL SEQUENCE (pRay451L)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGADFDLDLLPDFDLDLLPDFDLDLLPDFDLDLLPPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 71 - protein, 690 aa, Artificial sequence
  Synthetic polypeptide
  MISC_FEATURE: FULL SEQUENCE (pRay451M)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHMQNSHSGVNQLGGVFVNGRPLPDSTRQKIVELAHSGARPCDISRILQVSNGCVSKILGRYYETGSIRPRAIGGSKPRVATPEVVSKIAQYKRECPSIFAWEIRDRLLSEGVCTNDNIPSVSSINRVLRNLASEKQQMGADFDLDLLPDFDLDLLPDFDLDLLPDFDLDLLPDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 72 - protein, 8 aa, Artificial sequence
  Synthetic polypeptide
  MISC_FEATURE: Linker variant
GSGSGSGS

SEQ ID NO: 73 - protein, 21 aa, Artificial sequence
  Synthetic polypeptide
  MISC_FEATURE: CD8 alpha signal peptide
MALPVTALLLPLALLLHAARP

SEQ ID NO: 74 - protein, 10 aa, Artificial sequence
  Synthetic polypeptide
  MISC_FEATURE: Myc-tag
EQKLISEEDL

SEQ ID NO: 75 - protein, 5 aa, Artificial sequence
  Synthetic polypeptide
  MISC_FEATURE: STS variant 1
RKRRR

SEQ ID NO: 76 - protein, 1 aa, Artificial sequence
  Synthetic polypeptide
  MISC_FEATURE: STS variant 2
R

SEQ ID NO: 77 - protein, 2 aa, Artificial sequence
  Synthetic polypeptide
  MISC_FEATURE: STS variant 3
RK

SEQ ID NO: 78 - protein, 3 aa, Artificial sequence
  Synthetic polypeptide
  MISC_FEATURE: STS variant 4
RKR

SEQ ID NO: 79 - protein, 4 aa, Artificial sequence
  Synthetic polypeptide
  MISC_FEATURE: STS variant 5
RKRR

SEQ ID NO: 80 - protein, 8 aa, Artificial sequence
  Synthetic polypeptide
  MISC_FEATURE: RAM
QHGQLWFP

SEQ ID NO: 81 - protein, 1 aa, Artificial sequence
  Synthetic polypeptide
  MISC_FEATURE: RAM variant 1
Q

SEQ ID NO: 82 - protein, 2 aa, Artificial sequence
  Synthetic polypeptide
  MISC_FEATURE: RAM variant 2
QH

SEQ ID NO: 83 - protein, 3 aa, Artificial sequence
  Synthetic polypeptide
  MISC_FEATURE: RAM variant 3
QHG

SEQ ID NO: 84 - protein, 4 aa, Artificial sequence
  Synthetic polypeptide
  MISC_FEATURE: RAM variant 4
QHGQ

SEQ ID NO: 85 - protein, 5 aa, Artificial sequence
  Synthetic polypeptide
  MISC_FEATURE: RAM variant 5
QHGQL

SEQ ID NO: 86 - protein, 6 aa, Artificial sequence
  Synthetic polypeptide
  MISC_FEATURE: RAM variant 6
QHGQLW

SEQ ID NO: 87 - protein, 7 aa, Artificial sequence
  Synthetic polypeptide
  MISC_FEATURE: RAM variant 7
QHGQLWF

SEQ ID NO: 88 - protein, 9 aa, Artificial sequence
  Synthetic polypeptide
  MISC_FEATURE: RAM variant 8
QHGQLWFPE

SEQ ID NO: 89 - protein, 10 aa, Artificial sequence
  Synthetic polypeptide
  MISC_FEATURE: RAM variant 9
QHGQLWFPEG

SEQ ID NO: 90 - protein, 11 aa, Artificial sequence
  Synthetic polypeptide
  MISC_FEATURE: RAM variant 10
QHGQLWFPEGF

SEQ ID NO: 91 - protein, 7 aa, Artificial sequence
  Synthetic polypeptide
  MISC_FEATURE: NLS
PKKKRKV

SEQ ID NO: 92 - protein, 4 aa, Artificial sequence
  Synthetic polypeptide
  MISC_FEATURE: NLS 1
RRKK

SEQ ID NO: 93 - protein, 174 aa, Artificial sequence
  Synthetic polypeptide
  MISC_FEATURE: DBD (ZF6/SV40NLS)
SRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLR

SEQ ID NO: 94 - protein, 58 aa, Artificial sequence; identical to SEQ ID NO: 20
  Synthetic polypeptide
  MISC_FEATURE: TAD (human GR TAD1)

SEQ ID NO: 95 - protein, 53 aa, Artificial sequence
  Synthetic polypeptide
  MISC_FEATURE: TAD (human MIER3 TAD fragment)
DYDDERTLEEEEMMDEGKNFSSEIEDLEKEGTMPLEDLLAFYGYEPTIPAVAN

SEQ ID NO: 96 - protein, 53 aa, Artificial sequence
  Synthetic polypeptide
  MISC_FEATURE: TAD (human MLXIPL TAD fragment)
SDTLFTMTQSGPSPLQLPPEDAYVGNADMIQPDLTPLQPSLDDFMDISDFFTN

SEQ ID NO: 97 - protein, 53 aa, Artificial sequence
  Synthetic polypeptide
  MISC_FEATURE: TAD (human NFE2L1 TAD fragment)
RESETEQGFGEDLEDLGAVAPPVSGDLTKEDIDLIDILWRQDIDLGAGREVFD

SEQ ID NO: 98 - protein, 53 aa, Artificial sequence
  Synthetic polypeptide
  MISC_FEATURE: TAD human PTF1A TAD fragment
DAVLLEHFPGGLDAFPSSYFDEDDFFTDQSSRDPLEDGDELLADEQAEVEFLS

SEQ ID NO: 99 - protein, 700 aa, Artificial sequence
  Synthetic polypeptide
  MISC_FEATURE: FULL SEQUENCE (pDP1441)
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 100 - protein, 758 aa, Artificial sequence
  Synthetic polypeptide
  MISC_FEATURE: pDP1487
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSDQSTFDILQDLEFSSGSPGKETNESPWRSDLLIDENCLLSPLAGEDDSFLLEGNSNED

SEQ ID NO: 101 - protein, 753 aa, Artificial sequence
  Synthetic polypeptide
  MISC_FEATURE: pDP1558
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSDYDDERTLEEEEMMDEGKNFSSEIEDLEKEGTMPLEDLLAFYGYEPTIPAVAN

SEQ ID NO: 102 - protein, 753 aa, Artificial sequence
  Synthetic polypeptide
  MISC_FEATURE: pDP1560
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 103 - protein, 753 aa, Artificial sequence
  Synthetic polypeptide
  MISC_FEATURE: pDP1562
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSRESETEQGFGEDLEDLGAVAPPVSGDLTKEDIDLIDILWRQDIDLGAGREVFD

SEQ ID NO: 104 - protein, 753 aa, Artificial sequence
  Synthetic polypeptide
  MISC_FEATURE: pDP1566
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSDAVLLEHFPGGLDAFPSSYFDEDDFFTDQSSRDPLEDGDELLADEQAEVEFLS

SEQ ID NO: 105 - protein, 752 aa, Artificial sequence
  Synthetic polypeptide
  MISC_FEATURE: pDP1673
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 106 - protein, 760 aa, Artificial sequence
  Synthetic polypeptide
  MISC_FEATURE: pDP1674
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHGQLWFPPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 107 - protein, 759 aa, Artificial sequence
  Synthetic polypeptide
  MISC_FEATURE: pDP1675
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSKRKRKHGSLWLPPKKKRKVSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 108 - protein, 744 aa, Artificial sequence
  Synthetic polypeptide
  MISC_FEATURE: pDP1757
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 109 - protein, 745 aa, Artificial sequence
  Synthetic polypeptide
  MISC_FEATURE: pDP1758
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 110 - protein, 746 aa, Artificial sequence
  Synthetic polypeptide
  MISC_FEATURE: pDP1759
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 111 - protein, 747 aa, Artificial sequence
  Synthetic polypeptide
  MISC_FEATURE: pDP1760
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 112 - protein, 748 aa, Artificial sequence
  Synthetic polypeptide
  MISC_FEATURE: pDP1761
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 113 - protein, 750 aa, Artificial sequence
  Synthetic polypeptide
  MISC_FEATURE: pDP1762
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 114 - protein, 751 aa, Artificial sequence
  Synthetic polypeptide
  MISC_FEATURE: pDP1763
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 115 - protein, 752 aa, Artificial sequence
  Synthetic polypeptide
  MISC_FEATURE: pDP1764
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHGRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 116 - protein, 753 aa, Artificial sequence
  Synthetic polypeptide
  MISC_FEATURE: pDP1765
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHGQRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 117 - protein, 754 aa, Artificial sequence
  Synthetic polypeptide
  MISC_FEATURE: pDP1766
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHGQLRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 118 - protein, 755 aa, Artificial sequence
  Synthetic polypeptide
  MISC_FEATURE: pDP1767
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHGQLWRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 119 - protein, 756 aa, Artificial sequence
  Synthetic polypeptide
  MISC_FEATURE: pDP1768
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHGQLWFRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 120 - protein, 757 aa, Artificial sequence
  Synthetic polypeptide
  MISC_FEATURE: pDP1725
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHGQLWFPRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 121 - protein, 758 aa, Artificial sequence
  Synthetic polypeptide
  MISC_FEATURE: pDP1769
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHGQLWFPERRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 122 - protein, 759 aa, Artificial sequence
  Synthetic polypeptide
  MISC_FEATURE: pDP1770
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHGQLWFPEGRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 123 - protein, 760 aa, Artificial sequence
  Synthetic polypeptide
  MISC_FEATURE: pDP1771
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACFMYVAAAAFVLLFFVGCGVLLSRKRRRQHGQLWFPEGFRRKKSRPGERPFQCRICMRNFSQQTNLTRHTRTHTGEKPFQCRICMRNFSQKANLGVHLRTHTGSQKPFQCRICMRNFSDHSSLKRHTRTHTGEKPFQCRICMRNFSRADMLRRHLRTHTGSQKPFQCRICMRNFSEKQNLARHTRTHTGEKPFQCRICMRNFSDPSNLRRHLRTHLRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSSDTLFTMTQSGPSPLQLPPEDAYVGNADMIQPDLTPLQPSLDDFMDISDFFTN

SEQ ID NO: 124 - protein, 4 aa, Artificial sequence
  Synthetic polypeptide
GGGS

SEQ ID NO: 125 - protein, 4 aa, Artificial sequence
  Synthetic polypeptide
SGGG

SEQ ID NO: 126 - protein, 4 aa, Artificial sequence
  Synthetic polypeptide
SGSG

SEQ ID NO: 127 - protein, 5 aa, Artificial sequence
  Synthetic polypeptide
GGGGS

SEQ ID NO: 128 - protein, 5 aa, Artificial sequence
  Synthetic polypeptide
SGGGG

SEQ ID NO: 129 - protein, 6 aa, Artificial sequence
  Synthetic polypeptide
GGGGGS

SEQ ID NO: 130 - protein, 6 aa, Artificial sequence
  Synthetic polypeptide
SGGGGG

SEQ ID NO: 131 - protein, 7 aa, Artificial sequence
  Synthetic polypeptide
GGGGGGS

SEQ ID NO: 132 - protein, 7 aa, Artificial sequence
  Synthetic polypeptide
SGGGGGG

SEQ ID NO: 133 - protein, 250 aa, Artificial sequence
  Synthetic polypeptide
  MISC_FEATURE (1)..(250): Bases 1 to 250 may be present or absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 134 - protein, 250 aa, Artificial sequence
  Synthetic polypeptide
  MISC_FEATURE (1)..(250): Bases 1 to 250 may be present or absent
SGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGG

SEQ ID NO: 135 - protein, 25 aa, Artificial sequence
  Synthetic polypeptide
  MISC_FEATURE (1)..(20): Bases 1-20 may be present or absent
  MISC_FEATURE (1)..(20): Xaa is any amino acid that can be inserted into the sequence and not result in a polypeptide comprising the sequence GSG
GGGXXGGGXXGGGXXGGGXXGGGGS

SEQ ID NO: 136 - protein, 25 aa, Artificial sequence
  Synthetic polypeptide
  MISC_FEATURE (1)..(25): Xaa is any amino acid that can be inserted into the sequence and not result in a polypeptide comprising the sequence GSG
  MISC_FEATURE (6)..(25): Bases 6-25 may be present or absent
GGGGSXGGGSXGGGSXGGGSXGGGS

SEQ ID NO: 137 - protein, 25 aa, Artificial sequence
  Synthetic polypeptide
  MISC_FEATURE (1)..(20): Bases 1-20 may be present or absent
  MISC_FEATURE (4)..(4): Xaa is Proline
  MISC_FEATURE (5)..(5): Xaa is Serine
  MISC_FEATURE (9)..(9): Xaa is Proline
  MISC_FEATURE (10)..(10): Xaa is Serine
  MISC_FEATURE (14)..(14): Xaa is Proline
  MISC_FEATURE (15)..(15): Xaa is Serine
  MISC_FEATURE (19)..(19): Xaa is Proline
  MISC_FEATURE (20)..(20): Xaa is Serine
GGGXXGGGXXGGGXXGGGXXGGGGS

SEQ ID NO: 138 - protein, 25 aa, Artificial sequence
  Synthetic polypeptide
  MISC_FEATURE (1)..(20): Bases 1-20 may be present or absent
  MISC_FEATURE (4)..(4): Xaa is Glycine
  MISC_FEATURE (5)..(5): Xaa is Glutamine
  MISC_FEATURE (9)..(9): Xaa is Glycine
  MISC_FEATURE (10)..(10): Xaa is Glutamine
  MISC_FEATURE (14)..(14): Xaa is Glycine
  MISC_FEATURE (15)..(15): Xaa is Glutamine
  MISC_FEATURE (19)..(19): Xaa is Glycine
  MISC_FEATURE (20)..(20): Xaa is Glutamine
GGGXXGGGXXGGGXXGGGXXGGGGS

SEQ ID NO: 139 - protein, 25 aa, Artificial sequence
  Synthetic polypeptide
  MISC_FEATURE (1)..(20): Bases 1-20 may be present or absent
  MISC_FEATURE (4)..(4): Xaa is Glycine
  MISC_FEATURE (5)..(5): Xaa is Alanine
  MISC_FEATURE (9)..(9): Xaa is Glycine
  MISC_FEATURE (10)..(10): Xaa is Alanine
  MISC_FEATURE (14)..(14): Xaa is Glycine
  MISC_FEATURE (15)..(15): Xaa is Alanine
  MISC_FEATURE (19)..(19): Xaa is Glycine
  MISC_FEATURE (20)..(20): Xaa is Alanine
GGGXXGGGXXGGGXXGGGXXGGGGS

SEQ ID NO: 140 - protein, 25 aa, Artificial sequence
  Synthetic polypeptide
  MISC_FEATURE (6)..(25): Bases 6-25 may be present or absent
  MISC_FEATURE (6)..(6): Xaa is Proline
  MISC_FEATURE (11)..(11): Xaa is Proline
  MISC_FEATURE (16)..(16): Xaa is Proline
  MISC_FEATURE (21)..(21): Xaa is Proline
GGGGSXGGGSXGGGSXGGGSXGGGS

SEQ ID NO: 141 - protein, 15 aa, Artificial sequence
  Synthetic polypeptide
GGGGAGGGGAGGGGS

SEQ ID NO: 142 - protein, 15 aa, Artificial sequence
  Synthetic polypeptide
GGGGQGGGGQGGGGS

SEQ ID NO: 143 - protein, 15 aa, Artificial sequence
  Synthetic polypeptide
GGGPSGGGPSGGGGS

SEQ ID NO: 144 - protein, 15 aa, Artificial sequence
  Synthetic polypeptide
GGGGSPGGGSPGGGS

SEQ ID NO: 145 - protein, 4 aa, Artificial sequence
  Synthetic polypeptide
  MISC_FEATURE (2)..(3): Xaa can be any amino acid residue
  MISC_FEATURE (4)..(4): Xaa represents a hydrophobic residue such as Leu, Ile, Val, Phe, Trp, Tyr, Val, Met, and Pro
PXXX

SEQ ID NO: 146 - protein, 5 aa, Artificial sequence
  Synthetic polypeptide
  MISC_FEATURE (2)..(3): Xaa can be any amino acid residue
  MISC_FEATURE (4)..(4): Xaa represents a hydrophobic residue such as Leu, Ile, Val, Phe, Trp, Tyr, Val, Met, and Pro
  MISC_FEATURE (5)..(5): Xaa is either Serine or Threonine
PXXXX

SEQ ID NO: 147 - protein, 6 aa, Artificial sequence
  Synthetic polypeptide
  MISC_FEATURE (2)..(2): Xaa is either Leucine or Glutamine
PXGMTS

SEQ ID NO: 148 - protein, 5 aa, Artificial sequence
  Synthetic polypeptide
  MISC_FEATURE (2)..(2): Xaa is either Leucine or Glutamine
PXGMT

SEQ ID NO: 149 - protein, 7 aa, Artificial sequence
  Synthetic polypeptide
ENLYTQS

SEQ ID NO: 150 - protein, 5 aa, Artificial sequence
  Synthetic polypeptide
DDDDK

SEQ ID NO: 151 - protein, 4 aa, Artificial sequence
  Synthetic polypeptide
LVPR

SEQ ID NO: 152 - DNA, 20 bp, Artificial sequence
  Synthetic polypeptide
agacgtcgaagtagccgtag

SEQ ID NO: 153 - DNA, 20 bp, Artificial sequence
  Synthetic polypeptide
ggacgacgcggtctaagaag

SEQ ID NO: 154 - DNA, 20 bp, Artificial sequence
  Synthetic polypeptide
cggcgtagccgatgtcgcgc

SEQ ID NO: 155 - DNA, 22 bp, Artificial sequence
  Synthetic polypeptide
attttcacgcatgagtgcacag